(19) United States
(12) Patent Application Publication
(10) Pub. No.: US 2023/0391875 A1
(43) Pub. Date: Dec. 7, 2023

(54) DIAGNOSTIC AND THERAPEUTIC METHODS FOR CANCER

(71) Applicant: Genentech, Inc., South San Francisco, CA (US)

(72) Inventors: G Scott CHANDLER, Basel (CH); Zia KHAN, San Francisco, CA (US)

(21) Appl. No.: 18/167,295

(22) Filed: Feb. 10, 2023

Related U.S. Application Data
(63) Continuation of application No. PCT/US2021/045818, filed on Aug. 12, 2021.
(60) Provisional application No. 63/064,884, filed on Aug. 12, 2020.

(51) Int. Cl. C07K 16/28; C12Q 1/6886; A61K 45/06

(52) U.S. Cl. CPC C07K 16/2827; C12Q 1/6886; G01N 2800/52; G01N 2800/7028; A61K 45/06

(57) ABSTRACT

Provided herein are diagnostic and therapeutic methods for the treatment of cancer using polygenic risk scores (PRSs) for endocrine diseases, including hypothyroidism. In particular, the invention provides methods for patient selection and methods of treatment.

### CROSS-REFERENCES TO RELATED APPLICATIONS

This application is a continuation of International Patent Application No. PCT/US2021/045818, filed on Aug. 12, 2021, which claims priority to U.S. Patent Application No. 63/064,884, filed on Aug. 12, 2020, the entire contents of which are incorporated herein by reference in their entirety.

### SEQUENCE LISTING

The instant application contains a Sequence Listing which has been submitted electronically in XML format and is hereby incorporated by reference in its entirety. Said XML copy, created on Feb. 3, 2023, is named 50474-238003_Sequence_Listing_2_3_23.xml and is 59,159 bytes in size.

### FIELD OF THE INVENTION

Provided herein are diagnostic and therapeutic methods for the treatment of cancer using polygenic risk scores (PRSs) for thyroid disorders and methods for identifying patients who are likely to develop thyroid disorders under anti-cancer therapy. In particular, the invention provides methods for patient selection and methods of treatment.

### BACKGROUND

Cancer remains one of the most deadly threats to human health. In the U.S., cancer affects more than 1.7 million new patients each year and is the second leading cause of death after heart disease, accounting for approximately 1 in 4 deaths. It is also predicted that cancer may surpass cardiovascular diseases as the number one cause of death within 5 years.

Immune checkpoint inhibition has emerged as a promising treatment for some cancers, including cancers with high unmet need for treatment options. In healthy tissues, immune checkpoints function in the prevention of autoimmunity by limiting the activity of T-cells. Tumor cells may co-opt this mechanism to escape immune surveillance. Considerable attention has thus been given to therapies that suppress the function of immune checkpoints (e.g., immune checkpoint blockade) in patients having a cancer. A particular immune checkpoint protein of interest is programmed cell death protein-1 (PD-1 or CD279), which acts to limit the activity of T-cells in peripheral tissues. Blockade of PD-1 by a monoclonal antibody specific for PD-1 or its ligands, programmed death-ligand 1 (PD-L1; CD274) and programmed death-ligand 2 (PD-L2; CD273), has been shown to elicit durable anti-tumor responses in a subset of patients undergoing treatment for cancer.

Immune checkpoint inhibition has been associated with on-target toxicities that are referred to as immune-related adverse events (irAEs). Recent studies have observed a correlation between the occurrence of endocrine irAEs and longer patient survival under immune checkpoint inhibitor therapy. This correlation suggests that predisposition to endocrinopathies may positively affect outcomes for patients treated with immune checkpoint blockade. Luo et al., Clin. Cancer Res., first published Jul. 7, 2021), for example, present data relating to genetic risk of immunotherapy-mediated thyroid dysfunction and its impact on outcomes with PD-1 blockade in non-small cell lung cancer. However, irAEs occur only after immune checkpoint blockade therapy has been initiated.

Thus, there exists an unmet need for diagnostic approaches that enable the use of a patient's genetic predisposition to irAEs (e.g., hypothyroidism) to predict a favorable outcome from treatment with an immune checkpoint inhibitor (see, e.g., Khan et al., Nat. Commun., 12: Article number 3355, 2021, published on Jun. 7, 2021).

### SUMMARY OF THE INVENTION

In one aspect, the disclosure features a method of identifying an individual having a cancer who has an increased likelihood of experiencing treatment-induced thyroid dysfunction during treatment comprising an immune checkpoint inhibitor (e.g., a PD-L1 axis binding antagonist (e.g., a PD-L1 binding antagonist, e.g., atezolizumab)), the method comprising determining a polygenic risk score (PRS) for one or both of hypothyroidism and vitiligo from a sample from the individual, wherein (a) a PRS for hypothyroidism that is above a hypothyroidism reference PRS and/or (b) a PRS for vitiligo that is above a vitiligo reference PRS identifies the individual as one who may have an increased likelihood of experiencing treatment-induced thyroid dysfunction during treatment comprising an immune checkpoint inhibitor.

In another aspect, the disclosure features a method of treating an individual having a cancer, the method comprising (a) determining a PRS for one or both of hypothyroidism and vitiligo from a sample from the individual, wherein the PRS for hypothyroidism is above a hypothyroidism reference PRS and/or the PRS for vitiligo is above a vitiligo reference PRS; (b) administering an effective amount of an immune checkpoint inhibitor (e.g., a PD-L1 axis binding antagonist (e.g., a PD-L1 binding antagonist, e.g., atezolizumab)) to the individual; and (c) monitoring the individual for symptoms of thyroid dysfunction.

In some aspects, the cancer is metastatic urothelial carcinoma, non-squamous non-small cell lung cancer (NSCLC), small cell lung cancer (SCLC), renal cell carcinoma (RCC), or triple negative breast cancer (TNBC). In some aspects, the treatment comprising an immune checkpoint inhibitor is second-line (2L) treatment of metastatic urothelial carcinoma, first-line (1 L) treatment of NSCLC, or first-line (1 L) treatment of squamous NSCLC. In some aspects, the method comprises administering an immune checkpoint inhibitor as 2L treatment of metastatic urothelial carcinoma, 1 L treatment of NSCLC, or 1 L treatment of squamous NSCLC.

In another aspect, the disclosure features a method of identifying an individual having a triple-negative breast cancer (TNBC) who may benefit from a treatment comprising an immune checkpoint inhibitor (e.g., a PD-L1 axis binding antagonist (e.g., a PD-L1 binding antagonist, e.g., atezolizumab)), the method comprising determining a polygenic risk score (PRS) for hypothyroidism from a sample from the individual, wherein a PRS for hypothyroidism that is above a hypothyroidism reference PRS identifies the individual as one who may receive a benefit from the treatment comprising an immune checkpoint inhibitor.

In another aspect, the disclosure features a method for selecting a treatment for an individual having a TNBC, the method comprising determining a PRS for hypothyroidism from a sample from the individual, wherein a PRS for hypothyroidism that is above a hypothyroidism reference PRS identifies the individual as one who may receive a benefit from a treatment comprising an immune checkpoint inhibitor (e.g., a PD-L1 axis binding antagonist (e.g., a PD-L1 binding antagonist, e.g., atezolizumab)).

In some aspects, the PRS for hypothyroidism determined from the sample is above the hypothyroidism reference PRS, and the method further comprises administering to the individual an effective amount of an immune checkpoint inhibitor.

In some aspects, the PRS for hypothyroidism determined from the sample is below the hypothyroidism reference PRS.

In some aspects, the benefit is an increase in overall survival (OS).

In another aspect, the disclosure features a method of treating an individual having a TNBC, the method comprising (a) determining a PRS for hypothyroidism from a sample from the individual, wherein the PRS for hypothyroidism from the sample is above a hypothyroidism reference PRS; and (b) administering an effective amount of an immune checkpoint inhibitor (e.g., a PD-L1 axis binding antagonist (e.g., a PD-L1 binding antagonist, e.g., atezolizumab)) to the individual.

In another aspect, the disclosure features a method of treating an individual having a TNBC, the method comprising administering an immune checkpoint inhibitor (e.g., a PD-L1 axis binding antagonist (e.g., a PD-L1 binding antagonist, e.g., atezolizumab)) to the individual who has been determined to have a PRS for hypothyroidism that is above a hypothyroidism reference PRS.

In some aspects, the vitiligo reference PRS is a pre-assigned PRS.

In some aspects, the vitiligo reference PRS is a median PRS for vitiligo in a reference population. In some aspects, the reference population is a population of individuals having the cancer.

In some aspects, (a) the PRS for vitiligo of the sample from the individual or (b) the PRS for vitiligo of a sample from an individual in the reference population is calculated using the equation:

$$\hat{S} = {\sum }_{i = 1}^{M} {β}_{i} \cdot {G}_{i}$$

wherein (i) Ŝ is the PRS for vitiligo; (ii) M is the number of risk alleles selected from independent genetic signals in a genome-wide association study (GWAS) for vitiligo; (iii) i represents the index of a given SNP; (iv) βᵢ is the log odds ratio or conditionally independent odds ratio of the ith SNP; and (v) Gᵢ={0,1,2} is the number of copies of the SNP in the sample from the individual.

In some aspects, the risk alleles are selected from Table 7 and/or Table 8.

In some aspects, the risk alleles are identified in the sample by whole-genome sequencing.

In some aspects, the hypothyroidism reference PRS is a pre-assigned PRS.

In some aspects, the hypothyroidism reference PRS is a median PRS for hypothyroidism in a reference population. In some aspects, the reference population is a population of individuals having the cancer.

In some aspects, (a) the PRS for hypothyroidism of the sample from the individual or (b) the PRS for hypothyroidism of a sample from an individual in the reference population is calculated using the equation:

$$\hat{S} = {\sum }_{i = 1}^{M} {β}_{i} \cdot {G}_{i}$$

wherein (i) Ŝ is the PRS for hypothyroidism; (ii) M is the number of risk alleles selected from independent genetic signals in a genome-wide association study (GWAS) for hypothyroidism; (iii) i represents the index of a given SNP; (iv) βᵢ is the log odds ratio or conditionally independent odds ratio of the ith SNP; and (v) Gᵢ={0,1,2} is the number of copies of the SNP in the sample from the individual.

In some aspects, the risk alleles are selected from Table 7 and/or Table 8.

In some aspects, the risk alleles are identified in the sample by whole-genome sequencing.

In some aspects, the method further comprises assessing one or more properties that are positively associated with the predictive capacity of a PRS for hypothyroidism from a sample from the individual before administration of a treatment comprising an immune checkpoint inhibitor.

In some aspects, the property is a level of thyroid-stimulating hormone (TSH) that is above a TSH reference level.

In some aspects, the TSH reference level is a pre-assigned TSH level.

In some aspects, the TSH reference level is a median TSH level in the reference population.

In some aspects, the sample is a whole blood sample, a buccal swab, a plasma sample, a serum sample, a tissue biopsy, or a combination thereof. In some aspects, the sample is a whole blood sample.

In some aspects, the sample is an archival sample, a fresh sample, or a frozen sample.

In some aspects, the immune checkpoint inhibitor is a PD-L1 axis binding antagonist. In some aspects, the PD-L1 axis binding antagonist is a PD-L1 binding antagonist, a PD-1 binding antagonist, or a PD-L2 binding antagonist.

In some aspects, the PD-L1 axis binding antagonist is a PD-L1 binding antagonist.

In some aspects, the PD-L1 binding antagonist inhibits the binding of PD-L1 to one or more of its ligand binding partners. In some aspects, the PD-L1 binding antagonist inhibits the binding of PD-L1 to PD-1. In some aspects, the PD-L1 binding antagonist inhibits the binding of PD-L1 to B7-1. In some aspects, the PD-L1 binding antagonist inhibits the binding of PD-L1 to both PD-1 and B7-1.

In some aspects, the PD-L1 binding antagonist is an anti-PD-L1 antibody. In some aspects, the anti-PD-L1 antibody is selected from the group consisting of atezolizumab, MDX-1105, MEDI4736 (durvalumab), and MSB0010718C (avelumab). In some aspects, the anti-PD-L1 antibody is atezolizumab (MPDL3280A).

In some aspects, the PD-1 axis binding antagonist is a PD-1 binding antagonist.

In some aspects, the PD-1 binding antagonist inhibits the binding of PD-1 to one or more of its ligand binding partners. In some aspects, the PD-1 binding antagonist inhibits the binding of PD-1 to PD-L1. In some aspects, the PD-1 binding antagonist inhibits the binding of PD-1 to PD-L2. In some aspects, the PD-1 binding antagonist inhibits the binding of PD-1 to both PD-L1 and PD-L2.

In some aspects, the PD-1 binding antagonist is an anti-PD-1 antibody. In some aspects, the anti-PD-1 antibody is MDX 1106 (nivolumab), MK-3475 (pembrolizumab), MEDI-0680 (AMP-514), PDR001 (spartalizumab), REGN2810 (cemiplimab), BGB-108, prolgolimab, camrelizumab, sintilimab, tislelizumab, or toripalimab.

In some aspects, the PD-1 binding antagonist is an Fc-fusion protein. In some aspects, the Fc-fusion protein is AMP-224.

In some aspects, the PD-1 axis binding antagonist is a PD-L2 binding antagonist. In some aspects, the PD-L2 binding antagonist is an antibody or an immunoadhesin.

In some aspects, the immune checkpoint inhibitor is an anti-TIGIT antagonist antibody.

In some aspects, the anti-TIGIT antagonist antibody comprises the following hypervariable regions (HVRs): an HVR-H1 sequence comprising the amino acid sequence of SNSAAWN (SEQ ID NO: 34); an HVR-H2 sequence comprising the amino acid sequence of KTYYRFKWYSDYAVSVKG (SEQ ID NO: 35); an HVR-H3 sequence comprising the amino acid sequence of ESTTYDLLAGPFDY (SEQ ID NO: 36); an HVR-L1 sequence comprising the amino acid sequence of KSSQTVLYSSNNKKYLA (SEQ ID NO: 37); an HVR-L2 sequence comprising the amino acid sequence of WASTRES (SEQ ID NO: 38); and an HVR-L3 sequence comprising the amino acid sequence of QQYYSTPFT (SEQ ID NO: 39).

In some aspects, the anti-TIGIT antagonist antibody further comprises the following light chain variable region framework regions (FRs): an FR-L1 comprising the amino acid sequence of DIVMTQSPDSLAVSLGERATINC (SEQ ID NO: 40); an FR-L2 comprising the amino acid sequence of WYQQKPGQPPNLLIY (SEQ ID NO: 41); an FR-L3 comprising the amino acid sequence of GVPDRFSGSGSGTDFTLTISSLQAEDVAVYYC (SEQ ID NO: 42); and an FR-L4 comprising the amino acid sequence of FGPGTKVEIK (SEQ ID NO: 43).

In some aspects, the anti-TIGIT antagonist antibody further comprises the following heavy chain variable region FRs: an FR-H1 comprising the amino acid sequence of X₁VQLQQSGPGLVKPSQTLSLTCAISGDSVS (SEQ ID NO: 44), wherein X₁ is E or Q; an FR-H2 comprising the amino acid sequence of WIRQSPSRGLEWLG (SEQ ID NO: 45); an FR-H3 comprising the amino acid sequence of RITINPDTSKNQFSLQLNSVTPEDTAVFYCTR (SEQ ID NO: 46); and an FR-H4 comprising the amino acid sequence of WGQGTLVTVSS (SEQ ID NO: 47). In some aspects, X₁ is E. In some aspects, X₁ is Q.

In some aspects, the anti-TIGIT antagonist antibody comprises: (a) a heavy chain variable (VH) domain comprising an amino acid sequence having at least 95% sequence identity to the amino acid sequence of SEQ ID NO: 50 or 51; (b) a light chain variable (VL) domain comprising an amino acid sequence having at least 95% sequence identity to the amino acid sequence of SEQ ID NO: 52; or (c) a VH domain as in (a) and a VL domain as in (b).

In some aspects, the anti-TIGIT antagonist antibody comprises (a) a VH domain comprising the amino acid sequence of SEQ ID NO: 50 or 51; and (b) a VL domain comprising the amino acid sequence of SEQ ID NO: 52. In some aspects, the anti-TIGIT antagonist antibody comprises (a) a VH domain comprising the amino acid sequence of SEQ ID NO: 50; and (b) a VL domain comprising the amino acid sequence of SEQ ID NO: 52. In some aspects, the anti-TIGIT antagonist antibody comprises (a) a VH domain comprising the amino acid sequence of SEQ ID NO: 51; and (b) a VL domain comprising the amino acid sequence of SEQ ID NO: 52.

In some aspects, the anti-TIGIT antagonist antibody is a monoclonal antibody.

In some aspects, the anti-TIGIT antagonist antibody is a human antibody.

In some aspects, the anti-TIGIT antagonist antibody is a full-length antibody.

In some aspects, the anti-TIGIT antagonist antibody is tiragolumab.

In some aspects, the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

In some aspects, the anti-TIGIT antagonist antibody is an IgG class antibody. In some aspects, the IgG class antibody is an IgG1 subclass antibody.

In some aspects, the immune checkpoint inhibitor is an agent that targets one or more of CTLA-4, VISTA, B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, LAG-3, BTLA, IDO, OX40, and A2aR.

In some aspects, the method comprises administering at least two immune checkpoint inhibitors to the individual. In some aspects, the method comprises administering atezolizumab and tiragolumab to the individual.

In some aspects, the method further comprises administering to the individual one or more additional therapeutic agents.

In some aspects, the one or more additional therapeutic agents comprise an immunomodulatory agent, an anti-neoplastic agent, a chemotherapeutic agent, a growth inhibitory agent, an anti-angiogenic agent, a radiation therapy, a cytotoxic agent, a cellular therapy, or a combination thereof.

In some aspects, the one or more additional therapeutic agents comprise an effective amount of an anti-cancer therapy other than an immune checkpoint inhibitor.

In some aspects, the anti-cancer therapy is an anti-neoplastic agent, a chemotherapeutic agent, a growth inhibitory agent, an anti-angiogenic agent, a radiation therapy, a cytotoxic agent, or a cellular therapy.

In some aspects, the non-immune checkpoint inhibitor is an anti-neoplastic agent, a chemotherapeutic agent, a growth inhibitory agent, an anti-angiogenic agent, a radiation therapy, or a cytotoxic agent.

In some aspects, the non-immune checkpoint inhibitor is a chemotherapeutic agent. In some aspects, the chemotherapeutic agent is vinflunine, paclitaxel, or docetaxel.

In some aspects, the treatment comprising an immune checkpoint inhibitor is a monotherapy.

In some aspects, the individual has not been previously treated for the cancer.

In some aspects, the individual has not been previously administered an immune checkpoint inhibitor.

In some aspects, the individual is a human. In some aspects, the individual is female. In some aspects, the human is of European ancestry.

In another aspect, the disclosure features an immune checkpoint inhibitor (e.g., a PD-L1 axis binding antagonist (e.g., a PD-L1 binding antagonist, e.g., atezolizumab)) for use in treating an individual having a TNBC who has been identified as one who may benefit from a treatment comprising an immune checkpoint inhibitor based on a PRS for hypothyroidism from a sample from the individual that is above a hypothyroidism reference PRS.

In another aspect, the disclosure features use of an immune checkpoint inhibitor (e.g., a PD-L1 axis binding antagonist (e.g., a PD-L1 binding antagonist, e.g., atezolizumab)) in the manufacture of a medicament for treating an individual having a TNBC who has been identified as one who may benefit from a treatment comprising an immune checkpoint inhibitor based on a PRS for hypothyroidism from a sample from the individual that is above a hypothyroidism reference PRS.

In another aspect, the disclosure features a kit for identifying an individual having a cancer who has an increased likelihood of experiencing treatment-induced thyroid dysfunction during treatment comprising an immune checkpoint inhibitor (e.g., a PD-L1 axis binding antagonist (e.g., a PD-L1 binding antagonist, e.g., atezolizumab)), the kit comprising (a) polypeptides or polynucleotides for determining the presence of a set of risk alleles selected from independent genetic signals in a GWAS for hypothyroidism; and/or (b) polypeptides or polynucleotides capable of determining the presence of a set of risk alleles selected from independent genetic signals in a GWAS for vitiligo; and (c) instructions for use of the polypeptides or polynucleotides to determine a polygenic risk score (PRS) for one or both of hypothyroidism and vitiligo from a sample from the individual, wherein (i) a PRS for hypothyroidism that is above a hypothyroidism reference PRS or (ii) a PRS for vitiligo that is above a vitiligo reference PRS identifies the individual as one who may have an increased likelihood of experiencing treatment-induced thyroid dysfunction during treatment comprising a PD-L1 binding antagonist.

In another aspect, the disclosure features a kit for identifying an individual having a TNBC who may benefit from a treatment comprising an immune checkpoint inhibitor (e.g., a PD-L1 axis binding antagonist (e.g., a PD-L1 binding antagonist, e.g., atezolizumab)), the kit comprising (a) polypeptides or polynucleotides for determining the presence of a set of risk alleles selected from independent genetic signals in a GWAS for hypothyroidism; and (b) instructions for use of the polypeptides or polynucleotides to determine a polygenic risk score (PRS) for hypothyroidism from a sample from the individual, wherein a PRS for hypothyroidism that is above a hypothyroidism reference PRS identifies the individual as one who may benefit from a treatment comprising a PD-L1 binding antagonist.

In some aspects, the risk alleles are selected from Table 7 or Table 8.

In some aspects, the PD-L1 binding antagonist is atezolizumab (MPDL3280A).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a set of bar graphs showing the rate (fraction of patients) at which hypothyroidism and hyperthyroidism immune-related adverse events (irAEs) were observed in atezolizumab trials (anti-PD-L1) and their corresponding control arms (control). “Low” designates Common Terminology Criteria for Adverse Events (CTCAE) grading of 1 and 2. “High” designates CTCAE grade >2. Trial names are abbreviated as follows: imv211=IMvigor211; imp150=Impower150; imp131=Impower131; imp130=Impower130; imp133=Impower133; imm151=IMmotion151; impas130=IMpassion130. Abbreviations for treatment combinations are coded as follows: Atezo=atezolizumab monotherapy; A=atezolizumab; C=carboplatin; P=paclitaxel; NabP=Nab-paclitaxel; B=bevacizumab; SUN=sunitinib; E=etoposide.

FIG. 1B is a set of box-and-whisker plots showing the results of an individual participant data meta-analysis. Bars show the 95% confidence intervals around the hazard ratio (HR) for a time-dependent covariate in a Cox model associating occurrence of a given endocrine irAE and overall survival (OS) in patients treated with atezolizumab (N=3,552) or with standard of care treatments (N=2,523) in the control arms. Analysis included all safety evaluable patients. IPD meta-analysis p-values are provided for a two-sided Wald test that a logarithm (log) of the random effect estimate of a time-dependent covariate in a Cox model, stratified across trials, is non-zero for hypothyroidism p=5.26×10⁻¹⁵, hyperthyroidism p=1.02×10⁻⁴, type-1 diabetes p=0.06, adrenal insufficiency p=0.4, and hypophysitis p=0.93 in atezolizumab-treated patients. IPD meta-analysis p-values are provided for patients in the control arms for hypothyroidism p=0.0039, hyperthyroidism p=0.03, type-1 diabetes p=0.76, and adrenal insufficiency p=0.29 obtained by the same test. Subpanels to the right show the 95% confidence interval around the hazard ratio for this time-dependent covariate for hyperthyroidism and hypothyroidism split by each individual atezolizumab or chemotherapy combination trial arm. Trial arms and treatment combinations are abbreviated as described for FIG. 1A. *p<0.05, **p<0.01, ***p<0.001, ****p<0.0001.

FIG. 2A is a set of box-and-whisker plots showing individual participant data (IPD) meta-analysis hazard ratio (HR) expressed in unit normalized polygenic risk score (PRS) for the occurrence of hypothyroidism irAEs estimated in a mixed effect Cox model with genotype eigenvectors as fixed effect covariates using data from (N=1,584) atezolizumab-treated and (N=1,302) standard of care-treated European ancestry cancer patients across 7 clinical trials. PRSs are abbreviated as follows: hypoT=hypothyroidism; LDpred2=hypothyroidism PRS constructed by beta-shrinkage; T1D=type-1 diabetes; VIT=vitiligo. HRs were adjusted for genotype eigenvectors. IPD meta-analysis p-values are provided for a two-sided Wald test that the mixed effect Cox model estimated log-HR is non-zero for hypoT p=7.52×10⁻⁹, LDpred2 p=5.49×10⁻⁹, T1D p=0.67, and VIT p=1.10×10⁻⁶ in atezolizumab-treated patients and for hypoT p=0.68, T1D p=0.31, and VIT p=0.38 in the control arms stratified by arm. Subpanels to the right show the HR also expressed in unit PRS using a univariable model for each trial arm. Trial arms and treatment combinations are abbreviated as described for FIG. 1A. *p<0.05, **p<0.01, ***p<0.001, ****p<0.0001.

FIG. 2B is a cumulative incidence plot comparing the risk of hypothyroidism in patients treated with atezolizumab having polygenic risk scores for hypothyroidism (PRS(hypoT)) that are above or below the median. The median value was computed across all patients, including those in the control arms.

FIG. 2C is a cumulative incidence plot comparing the risk of hypothyroidism in the control arms of atezolizumab trials having polygenic risk scores for hypothyroidism (PRS(hypoT)) that are above or below the median. The median value was computed across all patients, including those in the control arms.

FIG. 2D is a box-and-whisker plot showing the random effect point estimate and 95% Cl for the IPD meta-analysis HR expressed in normalized unit PRS for the time to occurrence of hyperthyroidism irAEs using a mixed effect Cox model with genotype eigenvectors as fixed effect covariates in atezolizumab-treated (N=3,234) and standard of care-treated (N=2,297) cancer patients cancer patients of European ancestry across 7 clinical trials. Meta-analysis p-values are provided for a two-sided Wald test that the estimated log-HR is non-zero for hypoT p=0.016, T1D p=0.67, and VIT p=0.0012 in atezolizumab treated patients and hypoT p=0.57, T1D p=0.87, and VIT p=0.85 in standard of care treated patients stratified by trial arm. *p<0.05, **p<0.01, ***p<0.001, ****p<0.0001.

FIG. 2E is a bar graph showing the estimated importance of variants from the hypothyroidism PRS in a survival lasso model for time to hypothyroidism irAEs in atezolizumab treated patients. The genes whose transcription start sites (TSSs) are spanned by the credible set to which the lasso retained variant belongs are provided with no trailing parentheses. The two closest genes in genomic distance between credible set ends are indicated by trailing parentheses containing distance in kilobases (kb). Only genes who have a TSS within 500 kb are reported. Dash designates credible sets that span more than 3 TSSs.

FIG. 3A is a box-and-whisker plot showing the results of an IPD meta-analysis assessing the association between hypothyroidism irAEs and potential pre-treatment risk factors in a multivariable mixed effects Cox model fit to data from atezolizumab-treated (N=3,234) and standard of care-treated (N=2,297) cancer patients in the safety-evaluable population across the 7 clinical trials analyzed stratified across arms. Measurements were normalized across patients by normalization to the quantiles of a standard normal distribution and modelled as random effects. Poit estimates and 95% confidence interval (CI) for hazard ratios (HR) for hypothyroidism are expressed in unit normalized hormone levels after fitting the model. TSH=thyroid stimulating hormone; fT4=free thyroxine; fT3=free triiodothyronine. Gender is encoded as 1=female and 0=male. p-values are provided for a two-sided Wald test that the log-HR is non-zero for fT4 p=0.44, TSH p=9.93×10⁻¹⁴, and gender p=0.012 in atezolizumab-treated patients and fT4 p=0.25, TSH p=3.26×10⁻⁸, and gender p=0.00054 in standard of care-treated patients. Meta=analysis: *p<0.05, **p<0.01, ***p<0.001, ****p<0.0001.

FIG. 3B is a box-and-whisker plot showing meta-analysis hazard ratios (HRs) expressed in unit normalized polygenic risk score (PRS) for the occurrence of hypothyroidism. Random effect point estimate and 95% Cl are expressed in normalized unit PRS for the time to occurrence of hyperthyroidism irAEs estimated using a mixed effect Cox model with genotype eigenvectors as fixed effect covariates in the patient population described in FIG. 3A. TSHgwas uses a PRS constructed from a GWAS of thyroid-stimulating hormone (TSH) levels in individuals not receiving any medication for thyroid dysfunction. hypoT(adj) computes the association between hypothyroidism irAEs and the hypothyroidism PRS adjusted for baseline TSH levels and gender using these as fixed effect covariates in the model. In all cases, genotype eigenvectors were used as fixed effect covariates. Meta-analysis p-values are provided for a two-sided Wald test that the estimated log-HR is non-zero for hypoT(adj) p=3.91×10⁻⁷ and TSHgwas p=0.11 in atezolizumab-treated patients and hypoT(adj) p=0.66 and TSHgwas p=0.36 in the standard of care-treated patients stratified across trial arms. *p<0.05, **p<0.01, ***p<0.001, ****p<0.0001.

FIG. 3C is a cumulative incidence plot comparing risk of hypothyroidism in atezolizumab patients with all of the risk factors identified for hypothyroidism irAEs (PRS(hypoT) above median, female gender, and TSH above median) and with none of these risk factors (PRS(hypoT) below median, male gender, and TSH below median).

FIG. 3D is a plot showing positive predictive value and sensitivity (also known as precision and recall) for hypothyroidism irAEs and population hypothyroidism occurrence across thresholds for the PRS in atezolizumab-treated patients and estimated by 4-fold cross validation in the UK Biobank, respectively. Curves were also generated for subgroups that have increasing incidence of hypothyroidism irAEs on the basis of non-genetic risk factors. Meta-analysis: *p<0.05, **p<0.01, ***p<0.001, ****p<0.0001.

FIG. 4 is a Kaplan-Meier (KM) plot for overall survival (OS) for triple-negative breast cancer (TNBC) patients from the IMpassion130 trial treated with atezolizumab (A) and nab-paclitaxel (NabP). Patients were split into two groups on the basis of the median hypothyroidism PRS (HypoT) across all IMpassion130 patients with germline genetic data. High: above median; low: below median. Censoring events are denoted by dashes. Horizontal and vertical lines designate the median survival time.

FIG. 5 is a KM plot for OS for TNBC patients from the IMpassion130 trial treated with placebo plus nab-paclitaxel (NabP). Patients were split into two groups on the basis of the median hypothyroidism PRS (HypoT) across all IMpassion130 patients with germline genetic data. High: above median; low: below median. Censoring events are denoted by dashes. Horizontal and vertical lines designate the median survival time.

FIG. 6 is a set of bar graphs showing the rate (fraction of patients) at which rare endocrine irAEs (adrenal insufficiency, diabetes mellitus, and hypophysitis) were observed in atezolizumab trials (anti-PD-L1) and their corresponding control arms (control). Rates were computed in the entire safety evaluable population. High: Grade 3; Low: Grade 1-2. Trial arms and treatment combinations are abbreviated as described for FIG. 1A.

FIG. 7 is an incidence plot showing the frequency of rare endocrine irAEs (adrenal insufficiency, diabetes mellitus, and hypophysitis) across all trials for all safety evaluable patients.

FIG. 8 is a cumulative event plot for hypothyroidism and hyperthyroidism in all safety evaluable anti-PD-L1 treated patients.

FIG. 9 is a cumulative event plot for hypothyroidism comparing the atezolizumab and bevacizumab combination (AB) arm and the sunitinib (SUN) arm of the IMmotion151 trial in renal cell carcinoma (RCC).

FIG. 10 is a pair of graphs showing the properties of the 99% credible sets identified by fine mapping. Top: number of variants in each of 140 credible sets identified. Bottom: distribution of the posterior probability of association (PPA) of variants belonging to the credible sets.

FIG. 11 is a pair of matchstick plots showing −log₁₀ (p-values) for linkage disequilibrium (LD) score regression heritability enrichment for the UK Biobank (UKBB) hypothyroidism genome-wide association study (GWAS) (top panel) and a GWAS of TSH levels in accessible chromatin (bottom panel) measured by ATAC-seq across hematopoiesis. Orange circles designate enrichments significant at a false discovery rate (FDR) of 10%, as estimated by the Benjamini-Hochberg method. Cell types are coded as follows: C=common; P=progenitor; M=myeloid; Erythro=Erythrocyte; granulocyte/macrophage=GM; HSC=hematopoietic stem cell; LMP=lymphoid-primed multipotent; Mono=monocyte.

FIG. 12 is a bar graph showing the estimated importance of variants from the vitiligo PRS in a survival lasso model for time to hypothyroidism irAEs in atezolizumab-treated patients. The genes whose TSSs are spanned by the credible set to which the lasso retained variant belongs are provided with no trailing parentheses. The two closest genes in genomic distance between credible set ends are indicated by trailing parentheses containing distance in kilobases (kb). Only genes having a TSS within 500 kb are reported. “−” designates credible sets that span more than 3 TSSs.

FIG. 13 is a pair of chord plots illustrating promoter capture Hi-C interactions between variants retained in the hypothyroidism and vitiligo PRSs within the LPP gene. The transcription start sites of the LPP and BCL genes are highlighted. The positions of the PRS variants in LPP are shown by the red lines. Both rs6780858 (p=4.2×10⁻¹²) and rs2889896 (p=4.4×10⁻¹²) were eQTLs in blood for BCL6 in eQTLGen.

FIG. 14 is a box-and-whisker plot showing the results of an individual participant data meta-analysis assessing the association between hyperthyroidism irAEs and potential risk factors: thyroid hormone levels (TSH=thyroid stimulating hormone; fT4=free thyroxine; fT3=free triiodothyronine) and gender. Thyroid hormone levels were normalized across patients by normalization to the quantiles of a standard normal distribution. 95% Cl for hazard ratios for hyperthyroidism is expressed in unit normalized hormone levels. Gender is encoded as 1=female and 0=male. p-values are provided for a two-sided Wald test that the log-HR is non-zero for fT4 p=0.65, TSH p=0.11, and gender p=0.28 in atezolizumab treated patients and fT4 p=0.013, TSH p=0.032, and gender p=0.019 in standard of care-treated patients. Meta-analysis: *p<0.05, **p<0.01, ***p<0.001, ****p<0.0001.

FIG. 15A is a matchstick plot showing the stratified LD score regression enrichment (−log₁₀ (p-value) over the baseline model for ROADMAP Epigenomic and ENTex annotations for the 15 smallest enrichment p-values (out of 489 annotations tested) for the TSH GWAS.

FIG. 15B is a matchstick plot showing the enrichment p-values for the hypothyroidism GWAS from UKBB for the annotations described for FIG. 15A. Orange circles designate enrichments that are significant at a false discovery rate (FDR) of 10%, estimated using the Benjamini-Hochberg method.

FIG. 16A is a set of box-and-whisker plots showing the association between the hypothyroidism PRS and overall survival (OS) in the atezolizumab arms (left plot) and control arms (right plot) of the atezolizumab trials described in FIG. 1A. Estimates of the hazard ratios (HRs) are expressed in per unit PRS, which was normalized by quantile normalization to a standard normal distribution. Lines designate 95% confidence intervals around the estimated hazard ratios (center ticks). Hazard ratios were adjusted for genotype eigenvectors, baseline ECOG status and presence of pre-treatment liver metastases. p-values are provided for a two-sided Wald test that the log-HRs are significantly non-zero for the PRS coefficient in the Cox regression model. *p<0.05 (not significant after accounting for multiple testing), ****p<0.0001 (p-value significant after accounting multiple testing by Bonferroni correction). The association in the carboplatin and etoposide (CE) arm of IMpower133 did not meet the cutoff for multiple testing. *p<0.05, ****p<0.0001.

FIG. 16B is a set of box-and-whisker plots showing the association between the hypothyroidism PRS and progression-free survival (PFS) in the atezolizumab arms (left plot) and control arms (right plot) of the atezolizumab trials described in FIG. 1A. Estimates of the hazard ratios (HRs) are expressed in per unit PRS, which was normalized by quantile normalization to a standard normal distribution. Hazard ratios were adjusted for genotype eigenvectors, baseline ECOG status and presence of pre-treatment liver metastases. Lines designate 95% confidence intervals around the estimated hazard ratios (center ticks). p-values are provided for a two-sided Wald test that the log-HRs significantly non-zero for the PRS coefficient in the Cox regression model. *p<0.05 (not significant after accounting for multiple testing), ****p<0.0001 (p-value significant after accounting multiple testing by Bonferroni correction).

FIG. 16C is a set of box-and-whisker plots showing the association between the hypothyroidism PRS and best confirmed objective response by RECIST (0=progressive disease or stable disease, 1=partial response or complete response) in the atezolizumab arms (left plot) and control arms (right plot) of the atezolizumab trials described in FIG. 1A. Odds ratios (ORs) are expressed in per unit normalized PRS. Lines designate 95% confidence intervals. p-values are provided for a two-sided Wald test that the log-ORs are significantly non-zero for the PRS coefficient in the logistic regression model. *p<0.05 (not significant after accounting for multiple testing), ****p<0.0001 (p-value significant after accounting multiple testing by Bonferroni correction).

FIG. 17 is a KM plot for OS of TNBC patients from the atezolizumab plus nab-paclitaxel arm of IMpassion130. Patients were stratified on tumor PD-L1 positivity and by high (above median) and low (below median) hypothyroidism PRS. Dashed lines show censoring events. Vertical and horizontal lines designate the median survival time for each group. Abbreviations: pos=positive, neg=negative.

FIG. 18 is a consort diagram showing the reasons for removal of patients and whole genome sequencing data on the basis of informed consent, European ancestry, and quality control (QC) filters.

FIG. 19 is a cumulative incidence plot showing time to occurrence of hypothyroidism irAEs in atezolizumab-treated cancer patients that did not meet the cutoff for European ancestry (EUR<0.7). Patients were split by above-median or below-median hypothyroidism PRS values.

FIG. 20 is a Kaplan-Meier plot showing overall survival in TNBC patients from the atezolizumab plus nab-paclitaxel arm of IMpassion130 not meeting the cutoff for European ancestry (EUR<0.7). Patients were split by above-median or below-median hypothyroidism PRS values.

FIG. 21 is a pair of stacked bar graphs showing the fraction of patients with abnormal TSH measurements (TSH>5 mU/L) (Abn Lab), symptomatic hypothyroidism irAEs (Symp), or symptomatic hypothyroidism with an abnormal TSH measurement in the preceding 7 days (Lab Symp) in the indicated trial arms.

FIG. 22 is a pair of cumulative incidence plots showing risk of hypothyroidism in renal cell carcinoma patients from the IMmotion151 trial treated with atezolizumab and bevacizumab as compared to patients treated with sunitinib. Patients were split by above-median or below-median hypothyroidism PRS values. The median value was computed across all patients with genetic data, including those in the control arms.

FIG. 23 is a cumulative incidence plot for hypothyroidism in cancer patients in the control arms, excluding sunitinib-treated patients, stratified by above-median and below-median hypothyroidism PRS. The median value was computed across all patients with genetic data, including those in the control arms.

### DETAILED DESCRIPTION OF THE INVENTION

### I. Definitions

As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) aspects that are directed to that value or parameter per se. For example, description referring to “about X” includes description of “X.”

It is understood that aspects of the invention described herein include “comprising,” “consisting,” and “consisting essentially of” aspects.

As used herein, the term “adverse event” or “AE” refers to any unfavorable and unintended sign (including an abnormal laboratory finding), symptom, or disease temporally associated with the use of a medical treatment or procedure that may or may not be considered related to the medical treatment or procedure. Adverse events may be classified by “grade,” as defined by the National Cancer Institute Common Terminology Criteria for Adverse Events v5.0 (NIH CTCAE). In some aspects, the AE is a low grade AE, e.g., a Grade 1 or Grade 2 AE. Grade 1 includes AEs that are asymptomatic or have mild symptoms. Grade 2 includes AEs that are moderate and limit age-appropriate instrumental activities of daily living (e.g., preparing meals, shopping for groceries or clothes) and that indicate local or noninvasive intervention. In other instances, the AE is a high grade AE, e.g., a Grade 3, Grade 4, or Grade 5 AE. Grade 3 includes AEs that are severe or medically significant, but not immediately life-threatening, and that indicate hospitalization or prolongation of hospitalization. Grade 4 includes AEs that have life-threatening consequences and indicate urgent intervention. Grade 5 includes AEs that result in or relate to death.

As used herein, the term “immune-related adverse event” or “irAE” refers to an adverse event or “adverse event of special interest” (“AESI”), as classified by the NIH CTCAE, that has a putative immune-related etiology. In some aspects, the irAE is an AESI occurring as a result of immune checkpoint inhibitor therapy. In some aspects, the irAE affects the endocrine system (“endocrine irAE”), the skin (“dermatological irAE” or “skin irAE”), or the gastrointestinal tract (“GI irAE”). Endocrine irAEs include, but are not limited to, “immune-related hypothyroidism,” “immune-related hyperthyroidism,” “immune-related adrenal insufficiency,” “immune-related diabetes mellitus,” and “immune-related hypophysitis.” Dermatological irAEs include, but are not limited to, “immune-related rash” and “immune-related severe cutaneous reaction.” GI irAEs include, but are not limited to, “immune-related hepatitis,” “immune-related colitis,” and “immune-related pancreatitis.” In some aspects, the irAE is a low grade irAE, e.g., a Grade 1 AE (Grade 1 irAE) or Grade 2 AE (Grade 2 irAE).

As used herein, the term “vitiligo” refers to the physiological condition in mammals that is typically characterized by hypopigmentation, e.g., loss of skin or hair pigment, e.g., loss of melanocytes or of melanocyte function. In some aspects, skin hypopigmentation affects <10% of body surface area (Grade 1 hypopigmentation). In other aspects, skin hypopigmentation affects >10% of body surface area (Grade 2 hypopigmentation).

As used herein, the term “polygenic risk score” or “PRS” refers to a numerical value that reflects the number of single-nucleotide polymorphisms (SNPs) associated with an increased likelihood of developing a given pathological state, disease, or condition (e.g., an autoimmune condition, e.g., an endocrine autoimmune condition (e.g., hypothyroidism) or a dermatological autoimmune condition (e.g., vitiligo)) detected in a sample (e.g., a blood sample (e.g., a whole blood sample, a plasma sample, a serum sample, or a combination thereof), a buccal swab, or a tissue biopsy) obtained from an individual (e.g., an individual at risk of or having a cancer). The PRS can be measured, for example, on a whole genome basis, or on the basis of a subset of the genome (e.g., a predetermined set of loci, e.g., a set of loci in linkage disequilibrium). In some aspects, the predetermined set of loci does not comprise the entire genome. In some aspects, the predetermined set of loci comprise a plurality of loci at which one or more alleles are associated with an increased risk for the given pathological state, disease, or condition. In some aspects, the predetermined set of loci comprise at least about 5 or more, about 10 or more, about 20 or more, about 50 or more, about 100 or more, about 200 or more, about 500 or more, about 1000 or more, about 2000 or more, about 5000 or more, about 10,000 or more, about 15,000 or more, or about 20,000 or more loci.

As used herein, the term “reference polygenic risk score” or “reference PRS” refers to a PRS against which another PRS is compared, e.g., to make a diagnostic, predictive, prognostic, and/or therapeutic determination. For example, the reference PRS may be a PRS in a reference sample, a reference population, and/or a pre-determined value. In some aspects, the reference PRS is a cut-off value that significantly separates a first subset and a second subset of individuals who have been treated with an immune checkpoint inhibitor (e.g., a PD-L1 axis binding antagonist, e.g., a PD-L1 binding antagonist, e.g., atezolizumab) in the same reference population based on a significant difference between an individual's responsiveness to treatment with the immune checkpoint inhibitor, at or above the cut-off value or at or below the cut-off value. In some aspects, the individual's responsiveness to treatment with the immune checkpoint inhibitor (e.g., a PD-L1 axis binding antagonist, e.g., a PD-L1 binding antagonist, e.g., atezolizumab) therapy is significantly improved relative to the individual's responsiveness to treatment with the non-PD-L1 axis binding antagonist therapy at or above the cut-off value. In other aspects, the individual's responsiveness to treatment with the immune checkpoint inhibitor (e.g., a PD-L1 axis binding antagonist, e.g., a PD-L1 binding antagonist, e.g., atezolizumab) is significantly improved relative to the individual's responsiveness to treatment with the non-PD-L1 axis binding antagonist therapy at or below the cut-off value.

In some aspects, a reference PRS is defined as, e.g., the 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, or 75^{th} percentile of PRSs in the reference population. In some aspects, a reference PRS is defined as the 50^{th} percentile of PRSs in the reference population. In some aspects, a reference PRS is defined as the median of PRSs in the reference population.

The term “copy number of a gene” or “copy number of an allele” refers to the number of DNA loci in a cell having a particular sequence. Generally, for a given gene or locus, a mammal has two copies of each gene or locus. The copy number can be increased, e.g., by gene amplification or duplication, or reduced by deletion.

As used herein, the term “immune checkpoint inhibitor” refers to a therapeutic agent that targets at least one immune checkpoint protein to alter the regulation of an immune response, e.g., down-modulating, inhibiting, up-modulating, or activating an immune response. The term “immune checkpoint blockade” may be used to refer to a therapy comprising an immune checkpoint inhibitor. Immune checkpoint proteins are known in the art and include, without limitation, programmed cell death ligand 1 (PD-L1), TIGIT, cytotoxic T-lymphocyte antigen 4 (CTLA-4), programmed cell death 1 (PD-1), programmed cell death ligand 2 (PD-L2), V-domain Ig suppressor of T cell activation (VISTA), B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, LAG-3, BTLA, IDO, OX40, and A2aR. In some aspects, an immune checkpoint protein may be expressed on the surface of an activated T cell. Therapeutic agents that can act as immune checkpoint inhibitors useful in the methods of the present invention, include, but are not limited to, therapeutic agents that target one or more of PD-L1, TIGIT, PD-1, CTLA-4, PD-L2, VISTA, B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, LAG-3, BTLA, IDO, OX40, and A2aR. In some aspects, an immune checkpoint inhibitor enhances or suppresses the function of one or more targeted immune checkpoint proteins. In some aspects, the immune checkpoint inhibitor is a PD-L1 axis binding antagonist, such as atezolizumab, as described herein.

The term “PD-L1 axis binding antagonist” refers to a molecule that inhibits the interaction of a PD-L1 axis binding partner with either one or more of its binding partner, so as to remove T cell dysfunction resulting from signaling on the PD-1 signaling axis—with a result being to restore or enhance T cell function (e.g., proliferation, cytokine production, target cell killing). As used herein, a PD-L1 axis binding antagonist includes a PD-1 binding antagonist, a PD-L1 binding antagonist, and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some aspects, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one aspect, a PD-1 binding antagonist reduces the negative co-stimulatory signal mediated by or through cell surface proteins expressed on T lymphocytes mediated signaling through PD-1 so as render a dysfunctional T cell less dysfunctional (e.g., enhancing effector responses to antigen recognition). In some aspects, the PD-1 binding antagonist is an anti-PD-1 antibody. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In other aspects, a PD-1 binding antagonist is prolgolimab, camrelizumab, sintilimab, tislelizumab, or toripalimab.

The term “PD-L1 binding antagonist” refers to a molecule that decreases, blocks, inhibits, abrogates or interferes with signal transduction resulting from the interaction of PD-L1 with either one or more of its binding partners, such as PD-1 and B7-1. In some aspects, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some aspects, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and B7-1. In one aspect, a PD-L1 binding antagonist reduces the negative co-stimulatory signal mediated by or through cell surface proteins expressed on T lymphocytes mediated signaling through PD-L1 so as to render a dysfunctional T cell less dysfunctional (e.g., enhancing effector responses to antigen recognition). In some aspects, a PD-L1 binding antagonist is an anti-PD-L1 antibody. In still another specific aspect, an anti-PD-L1 antibody is atezolizumab, marketed as TECENTRIQ™ with a WHO Drug Information (International Nonproprietary Names for Pharmaceutical Substances), Recommended INN: List 74, Vol. 29, No. 3, 2015 (see page 387). In a specific aspect, an anti-PD-L1 antibody is MDX-1105. In another specific aspect, an anti PD-L1 antibody is MSB0015718C. In still another specific aspect, an anti-PD-L1 antibody is MEDI4736.

The term “PD-L2 binding antagonist” refers to a molecule that decreases, blocks, inhibits, abrogates or interferes with signal transduction resulting from the interaction of PD-L2 with either one or more of its binding partners, such as PD-1. In some aspects, a PD-L2 binding antagonist is a molecule that inhibits the binding of PD-L2 to one or more of its binding partners. In a specific aspect, the PD-L2 binding antagonist inhibits binding of PD-L2 to PD-1. In some aspects, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T cell less dysfunctional (e.g., enhancing effector responses to antigen recognition). In some aspects, a PD-L2 binding antagonist is an immunoadhesin.

Further examples of PD-L1 axis binding antagonists include cemiplimab, prolgolimab, camrelizumab, sintilimab, tislelizumab, toripalimab, dostarlimab, retifanlimab, spartalizumab, sasanlimab, penpulimab, CS1003, HLX10, SCT-110A, SHR-1316, CS1001, envafolimab, TQB2450, ZKAB001, LP-002, zimberelimab, balstilimab, genolimzumab, BI 754091, cetrelimab, YBL-006, BAT1306, HX008, CX-072, IMC-001, KL-A167, budigalimab, AMG 404, CX-188, JTX-4014, 609A, Sym021, LZM009, F520, SG001, APL-502, cosibelimab, lodapolimab, GS-4224, INCB086550, FAZ053, TG-1501, BGB-A333, BCD-135, AK-106, LDP, GR1405, HLX20, MSB2311, MAX-10181, RC98, BION-004, AM0001, CB201, ENUM 244C8, ENUM 388D4, AUNP-012, STI-1110, ADG104, AK-103, LBL-006, hAb21, AVA-004, PDL-GEX, INCB090244, KD036, KY1003, LYN192, MT-6035, VXM10, YBL-007, ABSK041, GB7003, JS-003, and HS-636.

The term “small molecule” refers to any molecule with a molecular weight of about 2000 daltons or less, preferably of about 500 daltons or less.

The term “antibody” herein is used in the broadest sense and encompasses various antibody structures, including but not limited to monoclonal antibodies, polyclonal antibodies, multispecific antibodies (e.g., bispecific antibodies), and antibody fragments (e.g., bis-Fabs) so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to bis-Fabs; Fv; Fab; Fab, Fab′-SH; F(ab′)₂; diabodies; linear antibodies; single-chain antibody molecules (e.g., scFv, scFab); and multispecific antibodies formed from antibody fragments.

“Single-chain Fv” or “scFv” antibody fragments comprise the VH and VL domains of an antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of antibodies are called α, δ, ε, γ, and μ, respectively.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain aspects, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg et al., Intern. Rev. Immunol. 13: 65-93 (1995)).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter. J. Mol. Biol. 227:381, 1991; Marks et al. J. Mol. Biol. 222:581, 1991. Also available for the preparation of human monoclonal antibodies are methods described in Cole et al. Monoclonal Antibodies and Cancer Therapy, Alan R. Liss, p. 77 (1985); Boerner et al. J. Immunol., 147(1):86-95,1991. See also van Dijk and van de Winkel. Curr. Opin. Pharmacol. 5:368-74, 2001. Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al. Proc. Natl. Acad. Sci. USA. 103:3557-3562, 2006 regarding human antibodies generated via a human B-cell hybridoma technology.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al. supra. In one aspect, for the VH, the subgroup is subgroup III as in Kabat et al. supra.

A “humanized” antibody refers to a chimeric antibody comprising amino acid residues from non-human HVRs and amino acid residues from human FRs. In certain aspects, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3).

The terms “anti-PD-L1 antibody” and “an antibody that binds to PD-L1” refer to an antibody that is capable of binding PD-L1 with sufficient affinity such that the antibody is useful as a diagnostic and/or therapeutic agent in targeting PD-L1. In one aspect, the extent of binding of an anti-PD-L1 antibody to an unrelated, non-PD-L1 protein is less than about 10% of the binding of the antibody to PD-L1 as measured, for example, by a radioimmunoassay (RIA). In some aspects, an anti-PD-L1 antibody binds to an epitope of PD-L1 that is conserved among PD-L1 from different species. In another aspect, an anti-PD-L1 antibody is atezolizumab, marketed as TECENTRIQ™ with a WHO Drug Information (International Nonproprietary Names for Pharmaceutical Substances), Recommended INN: List 74, Vol. 29, No. 3, 2015 (see page 387).

The terms “anti-PD-1 antibody” and “an antibody that binds to PD-1” refer to an antibody that is capable of binding PD-1 with sufficient affinity such that the antibody is useful as a diagnostic and/or therapeutic agent in targeting PD-1. In one aspect, the extent of binding of an anti-PD-1 antibody to an unrelated, non-PD-1 protein is less than about 10% of the binding of the antibody to PD-1 as measured, for example, by a radioimmunoassay (RIA). In certain aspects, an anti-PD-1 antibody binds to an epitope of PD-1 that is conserved among PD-1 from different species.

A “blocking” antibody or an “antagonist” antibody is one which inhibits or reduces biological activity of the antigen it binds. In some aspects, blocking antibodies or antagonist antibodies substantially or completely inhibit the biological activity of the antigen.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In some aspects, the extent of binding of an antibody to an unrelated target is less than about 10% of the binding of the antibody to the target as measured, e.g., by a radioimmunoassay (RIA). In some aspects, an antibody that specifically binds to a target has a dissociation constant (K_{D}) of ≤1 μM, ≤100 nM, ≤10 nM, ≤1 nM, or ≤0.1 nM. In some aspects, an antibody specifically binds to an epitope on a protein that is conserved among the protein from different species. In other aspects, specific binding can include, but does not require exclusive binding.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “biomarker” as used herein refers to an indicator, e.g., predictive, diagnostic, and/or prognostic, which can be detected in a sample, e.g., a single-nucleotide polymorphism (SNP), or derived therefrom (e.g., a PRS). In some aspects, a biomarker is a genetic locus, a collection of genetic loci, or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers. The biomarker may serve as an indicator of the likelihood of developing a given pathological state, disease, or condition (e.g., an autoimmune condition, e.g., a dermatological autoimmune condition, e.g., vitiligo, psoriasis, or atopic dermatitis), or of developing a particular subtype of a disease or disorder (e.g., cancer) characterized by certain, molecular, pathological, histological, and/or clinical features (e.g., responsiveness to therapy including an immune checkpoint inhibitor).

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, buccal swab, sputum, tears, perspiration, mucus, tumor lysates, and tissue culture medium, tissue extracts such as homogenized tissue, tumor tissue, cellular extracts, and combinations thereof. The sample may be an archival sample, a fresh sample, or a frozen sample. In some instances, the sample is a buccal swab, whole blood sample, a plasma sample, a serum sample, or a combination thereof.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes.

The term “survival” refers to the patient remaining alive, and includes overall survival as well as progression-free survival.

As used herein, “progression-free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall survival” or “OS” refers to the percentage of individuals in a group who are likely to be alive after a particular duration of time.

By “extending survival” is meant increasing overall survival and/or progression-free survival in a treated patient relative to an untreated patient (i.e. relative to a patient not treated with the medicament), or relative to a patient who has been treated with a non-immune checkpoint inhibitor therapy, wherein the patient having extended survival (e.g., overall survival) has a PRS for hypothyroidism that is above a hypothyroidism reference PRS and/or a PRS for vitiligo that is above a vitiligo reference PRS.

As used herein, “hazard ratio” or “HR” is a statistical definition for rates of events. For the purpose of the invention, hazard ratio is defined as representing the probability of an event (e.g., PFS or OS) in the experimental (e.g., treatment) group/arm divided by the probability of an event in the control group/arm at any specific point in time. An HR with a value of 1 indicates that the relative risk of an endpoint (e.g., death) is equal in both the “treatment” and “control” groups; a value greater than 1 indicates that the risk is greater in the treatment group relative to the control group; and a value less than 1 indicates that the risk is greater in the control group relative to the treatment group. “Hazard ratio” in progression-free survival analysis (i.e., PFS HR) is a summary of the difference between two progression-free survival curves, representing the reduction in the risk of death on treatment compared to control, over a period of follow-up. “Hazard ratio” in overall survival analysis (i.e., OS HR) is a summary of the difference between two overall survival curves, representing the reduction in the risk of death on treatment compared to control, over a period of follow-up.

The word “label” when used herein refers to a compound or composition that is conjugated or fused directly or indirectly to a reagent such as a polynucleotide probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable. The term is intended to encompass direct labeling of a probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

An “effective amount” of a compound, for example, an immune checkpoint inhibitor (e.g., a PD-L1 axis binding antagonist, e.g., a PD-L1 binding antagonist, e.g., atezolizumab) or a composition (e.g., pharmaceutical composition) thereof, is at least the minimum amount required to achieve the desired therapeutic or prophylactic result, such as a measurable improvement or prevention of a particular disorder (e.g., a cell proliferative disorder, e.g., cancer). An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

A “disorder” is any condition that would benefit from treatment including, but not limited to, chronic and acute disorders or diseases including those pathological conditions which predispose the mammal to the disorder in question. In one aspect, the disorder is a cancer.

The terms “cancer” and “cancerous” refer to or describe the physiological condition in mammals that is typically characterized by unregulated cell growth/proliferation. Aspects of cancer include solid tumor cancers and non-solid tumor cancers. Solid cancer tumors include, but are not limited to a breast cancer, a bladder cancer, a lung cancer, a kidney cancer, a melanoma, a colorectal cancer, a head and neck cancer, an ovarian cancer, a pancreatic cancer, or a prostate cancer, or metastatic forms thereof. In some aspects, the cancer is a bladder cancer. In some aspects, the cancer is a breast cancer. Further aspects of breast cancer include a triple-negative breast cancer (TNBC). Other aspects of breast cancer include a hormone receptor-positive (HR+) breast cancer, e.g., an estrogen receptor-positive (ER+) breast cancer, a progesterone receptor-positive (PR+) breast cancer, or an ER+/PR+ breast cancer. Yet other aspects of breast cancer include a HER2-positive (HER2+) breast cancer. In some aspects, the breast cancer is an early breast cancer. In some aspects, the cancer is a bladder cancer. Further aspects of bladder cancer include urothelial carcinoma (UC), muscle invasive bladder cancer (MIBC), and non-muscle invasive bladder cancer (NMIBC). In some aspects, the bladder cancer is a metastatic urothelial carcinoma (mUC). In some aspects, the mUC is a second-line (2L) mUC. In some aspects, the cancer is a lung cancer. Further aspects of lung cancer include an epidermal growth factor receptor-positive (EGFR+) lung cancer. Other aspects of lung cancer include an epidermal growth factor receptor-negative (EGFR−) lung cancer. Yet other aspects of lung cancer include a non-small cell lung cancer (NSCLC), e.g., a squamous lung cancer or a non-squamous lung cancer. In some aspects, the NSCLC is a first-line (1 L) non-squamous NSCLC or a 1 L squamous NSCLC. Other aspects of lung cancer include a small cell lung cancer (SCLC). In some aspects, the cancer is a kidney cancer. Further aspects of kidney cancer include a renal cell carcinoma (RCC). In some aspects, the cancer is a head and neck cancer. Further aspects of head and neck cancer include a squamous cell carcinoma of the head and neck (SCCHN). In some aspects, the cancer is a liver cancer. Further aspects of liver cancer include a hepatocellular carcinoma. In some aspects, the cancer is a prostate cancer. Further aspects of prostate cancer include a castration-resistant prostate cancer (CRPC). In some aspects, the cancer is a metastatic form of a solid tumor. In some aspects, the metastatic form of a solid tumor is a metastatic form of a melanoma, a breast cancer, a colorectal cancer, a lung cancer, a head and neck cancer, a bladder cancer, a kidney cancer, an ovarian cancer, a pancreatic cancer, or a prostate cancer. In some aspects, the cancer is a non-solid tumor cancer. Non-solid tumor cancers include, but are not limited to, a B-cell lymphoma. Further aspects of B-cell lymphoma include, e.g., a chronic lymphocytic leukemia (CLL), a diffuse large B-cell lymphoma (DLBCL), a follicular lymphoma, myelodysplastic syndrome (MDS), a non-Hodgkin lymphoma (NHL), an acute lymphoblastic leukemia (ALL), a multiple myeloma, an acute myeloid leukemia (AML), or a mycosis fungoides (MF).

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, an immune checkpoint inhibitor is used to delay development of a disease or to slow the progression of a disease.

The term “anti-cancer therapy” refers to a therapy useful in treating cancer. Examples of anti-cancer therapeutic agents include, but are not limited to, cytotoxic agents, chemotherapeutic agents, growth inhibitory agents, agents used in radiation therapy, anti-angiogenesis agents, apoptotic agents, anti-tubulin agents, and other agents to treat cancer, for example, anti-CD20 antibodies, platelet derived growth factor inhibitors (e.g., GLEEVEC™ (imatinib mesylate)), a COX-2 inhibitor (e.g., celecoxib), interferons, cytokines, antagonists (e.g., neutralizing antibodies) that bind to one or more of the following targets PDGFR-p, BlyS, APRIL, BCMA receptor(s), TRAIL/Apo2, other bioactive and organic chemical agents, and the like. Combinations thereof are also included in the invention.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anti-cancer agents disclosed below.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5c-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free) (nab-paclitaxel), albumin-engineered nanoparticle formulations of paclitaxel (American Pharmaceutical Partners, Schaumberg, III.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 A antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11 F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), interleukin 1 (IL-1) blockers such as anakinra (Kineret), T cell costimulation blockers such as abatacept (Orencia), interleukin 6 (IL-6) blockers such as tocilizumab (ACTEMERA®); interleukin 13 (IL-13) blockers such as lebrikizumab; interferon alpha (IFN) blockers such as Rontalizumab; beta 7 integrin blockers such as rhuMAb Beta7; IgE pathway blockers such as Anti-M1 prime; Secreted homotrimeric LTa3 and membrane bound heterotrimer LTa1/β2 blockers such as anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

A “growth inhibitory agent” when used herein refers to a compound or composition which inhibits growth of a cell either in vitro or in vivo. In one aspect, growth inhibitory agent is growth inhibitory antibody that prevents or reduces proliferation of a cell expressing an antigen to which the antibody binds. In another aspect, the growth inhibitory agent may be one which significantly reduces the percentage of cells in S phase. Aspects of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

A “subject” or an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the subject or individual is a human.

As used herein, “administering” is meant a method of giving a dosage of a compound (e.g., an immune checkpoint inhibitor) to a subject. In some aspects, the compositions utilized in the methods herein are administered intravenously. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer, for example, to the symptoms of the disorder being treated, the presence or size of metastases, or the size of the primary tumor.

The term “TIGIT” or “T-cell immunoreceptor with Ig and ITIM domains” as used herein refers to any native TIGIT from any vertebrate source, including mammals such as primates (e.g., humans) and rodents (e.g., mice and rats), unless otherwise indicated. TIGIT is also known in the art as DKFZp667A205, FLJ39873, V-set and immunoglobulin domain-containing protein 9, V-set and transmembrane domain-containing protein 3, VSIG9, VSTM3, and WUCAM. The term encompasses “full-length,” unprocessed TIGIT (e.g., full-length human TIGIT having the amino acid sequence of SEQ ID NO: 53), as well as any form of TIGIT that results from processing in the cell (e.g., processed human TIGIT without a signal sequence, having the amino acid sequence of SEQ ID NO: 54). The term also encompasses naturally occurring variants of TIGIT, e.g., splice variants or allelic variants. The amino acid sequence of an exemplary human TIGIT may be found under UniProt Accession Number Q495A1.

The term “anti-TIGIT antagonist antibody” refers to an antibody or an antigen-binding fragment or variant thereof that is capable of binding TIGIT with sufficient affinity such that it substantially or completely inhibits the biological activity of TIGIT. For example, an anti-TIGIT antagonist antibody may block signaling through PVR, PVRL2, and/or PVRL3 so as to restore a functional response by T-cells (e.g., proliferation, cytokine production, target cell killing) from a dysfunctional state to antigen stimulation. For example, an anti-TIGIT antagonist antibody may block signaling through PVR without impacting PVR-CD226 interaction. It will be understood by one of ordinary skill in the art that in some instances, an anti-TIGIT antagonist antibody may antagonize one TIGIT activity without affecting another TIGIT activity. For example, an anti-TIGIT antagonist antibody for use in certain of the methods or uses described herein is an anti-TIGIT antagonist antibody that antagonizes TIGIT activity in response to one of PVR interaction, PVRL3 interaction, or PVRL2 interaction, e.g., without affecting or minimally affecting any of the other TIGIT interactions. In one embodiment, the extent of binding of an anti-TIGIT antagonist antibody to an unrelated, non-TIGIT protein is less than about 10% of the binding of the antibody to TIGIT as measured, e.g., by a radioimmunoassay (RIA). In certain embodiments, an anti-TIGIT antagonist antibody that binds to TIGIT has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g. from 10⁻⁸ M to 10⁻¹³ M, e.g., from 10⁻⁹ M to 10⁻¹³ M). In certain embodiments, an anti-TIGIT antagonist antibody binds to an epitope of TIGIT that is conserved among TIGIT from different species or an epitope on TIGIT that allows for cross-species reactivity. In one embodiment, the anti-TIGIT antagonist antibody is tiragolumab.

As used herein, the term “tiragolumab” refers to an anti-TIGIT antagonist antibody having the International Nonproprietary Names for Pharmaceutical Substances (INN) List 117 (WHO Drug Information, Vol. 31, No. 2, 2017, p. 343), or the CAS Registry Number 1918185-84-8. Tiragolumab is also interchangeably referred to as “R07092284.”

### II. Predictive Methods and Assays

The invention is based, at least in part, on the discovery that determining a polygenic risk score (PRS) for hypothyroidism can be used as a biomarker (e.g., a predictive biomarker) in the treatment of an individual having a breast cancer (e.g., a triple-negative breast cancer (TNBC)), e.g., for determining whether an individual having such a cancer is likely to respond to treatment with an anti-cancer therapy that includes an immune checkpoint inhibitor (e.g., a PD-L1 axis binding antagonist (e.g., a PD-L1 binding antagonist, e.g., atezolizumab)) and/or an anti-TIGIT antagonist antibody) or for selecting a therapy for an individual having a cancer. In some aspects, a high PRS for hypothyroidism is associated with increased likelihood of response to treatment with an immune checkpoint inhibitor.

Additionally, the invention is based, at least in part, on the discovery that a PRS for hypothyroidism or vitiligo can be used as a biomarker (e.g., a predictive biomarker) in the treatment of an individual having a cancer, e.g., for determining whether an individual having such a cancer is likely to experience treatment-induced thyroid dysfunction during treatment with an anti-cancer therapy that includes an immune checkpoint inhibitor (e.g., a PD-L1 axis binding antagonist (e.g., a PD-L1 binding antagonist, e.g., atezolizumab) and/or an anti-TIGIT antagonist antibody) or for selecting a therapy for an individual having a cancer. In some aspects, a high PRS for hypothyroidism or vitiligo is associated with increased likelihood of experiencing treatment-related thyroid dysfunction during treatment with an immune checkpoint inhibitor.

Accordingly, also provided herein are methods and assays of evaluating PRSs for hypothyroidism and vitiligo in a sample from an individual. Any of the methods provided herein may include administering an anti-cancer therapy other than, or in additional to, an immune checkpoint inhibitor to the individual. Any of the methods may further include administering an effective amount of an additional therapeutic agent, as described herein, to the individual.

A. Diagnostic Methods and Assays

i. Methods of Determining Polygenic Risk Scores (PRSs)

Ia. Identification of Risk Alleles

In some aspects, the invention features methods that include determining one or more polygenic risk scores (PRSs) of an individual for one or more endocrine or dermatological autoimmune diseases, e.g., hypothyroidism or vitiligo. PRS may be represented as the number of single-nucleotide polymorphisms (SNPs) associated with increased likelihood of having or developing a disease, state, or condition (“risk alleles”), e.g., hypothyroidism risk alleles or vitiligo risk alleles counted over a defined number of sequenced base pairs or in the whole genome sequence of an individual.

Risk alleles may be identified using a number of methods. In some aspects, risk alleles may be identified in a genome-wide association study (GWAS) for a pathological state, disease, or condition of interest. In some aspects, individuals included in the GWAS may be clinically diagnosed as having the disease, state, or condition, e.g., diagnosed using the International Classification of Diseases (ICD) code. In other aspects, individuals included in the GWAS may self-identify as having the disease, state, or condition. Exemplary GWAS for TSH levels, hypothyroidism, Type 1 diabetes, and vitiligo are reported in Table 1. GWAS may identify one or more genic or non-genic loci (e.g., a SNP), e.g., 1 or more loci, 5 or more loci, 10 or more loci, 15 or more loci, 20 or more loci, 25 or more loci, 30 or more loci, 40 or more loci, 50 or more loci, 60 or more loci, 70 or more loci, 80 or more loci, 90 or more loci, 100 or more loci, 150 or more loci, 200 or more loci, 300 or more loci, 400 or more loci, 500 or more loci, 1000 or more loci, 2000 or more loci, 3000 or more loci, 4000 or more loci, 5000 or more loci, 10,000 or more loci, 50,000 or more loci, 100,000 or more loci, 200,000 or more loci, or 500,000 or more loci to be included in the set of risk alleles. The GWAS p-value threshold at which the PRS is most predictive is often unknown, and PRSs may use SNPs that do not achieve genome-wide significant p-values in the original GWAS (Dudbridge, PLoS Genet., 9: e1003348, 2013; Euesden et al., Bioinformatics, 31: 1466-1468, 2015). The p-value threshold for inclusion in the set of risk alleles may be, e.g., p<0.2, p<0.1, p<0.05, p<0.01, p<0.001, p<1×10⁻⁴, p<1×10⁻⁵s, p<1×10⁻⁶, p<1×10⁻⁷, p<1×10⁻⁸, p<1×10⁻⁹, or p<1×10⁻¹⁰.

TABLE 1
GWAS summary statistics analyzed

| Trait | Abbreviation | GWAS | Cases/Controls | Citation |
|---|---|---|---|---|
| Thyroid | TSHgwas | TSH levels, | 54288/NA | Teumer et |
| stimulating | | excluding | | al., Nat. |
| hormone | | patients receiving | | Commun., 9: |
| | | medication for | | 4455, 2018. |
| | | thyroid | | |
| | | dysfunction | | |
| Hypothyroidism | hypoT | ICD Code + Self | 25072/383887 | Bycroft et al., |
| | | Reported | | Nature, 562: |
| | | Hypothyroidism | | 203-209, |
| | | UKBioBank | | 2018. |
| Type 1 | T1D | Type-1 Diabetes | 5913/8828 | Censin et al., |
| diabetes | | Meta-analysis | | PLoS Med., |
| | | | | 14: |
| | | | | e1002362, |
| | | | | 2017. |
| Vitiligo | VIT | Vitiligo GWAS | 4680/39586 | Jin et al., Nat. |
| | | | | Genet., 48: |
| | | | | 1418-1424, |
| | | | | 2016. |

NA = not applicable

The hypothyroidism GWAS was conducted using SAIGE (Zhou et al., Nat. Genet., 50: 1335-1341, 2018). The genomic inflation factor for the hypoT GWAS was λ_{gc}=1.192. As λ_{gc} scales with sample size, the inflation factor was computed for an equivalent study of 1000 cases and 1000 controls λ₁₀₀₀=1.004 confirming no significant test statistic inflation (de Bakker et al., Hum. Mol. Genet., 17: R122-128, 2008).

In some aspects, the GWAS may identify risk alleles for TSH levels (e.g., as described in Teumer et al., Nat. Commun., 9: 4455, 2018). In other aspects, the GWAS may identify risk alleles for hypothyroidism (e.g., as described in Bycroft et al., Nature, 562: 203-209, 2018). In other aspects, the GWAS may identify risk alleles for T1 D (e.g., as described in Censin et al., PLoS Med., 14: e1002362, 2017). In yet other aspects, the GWAS may identify risk alleles for vitiligo (e.g., as described in Jin et al., Nat. Genet., 48: 1418-1424, 2016).

In some aspects, the GWAS for vitiligo identifies 10 or more, 20 or more, 30 or more, 40 or more, 50 or more, 100 or more, 200 or more, 300 or more, 400 or more, 500 or more, 1000 or more, 2000 or more, 3000 or more, 4000 or more, 5000 or more, 6,000 or more, 10,000 or more, 15,000 or more, 25,000 or more, 50,000 or more, 100,000 or more, 150,000 or more, or 200,000 or more risk alleles for vitiligo. In some aspects, the GWAS for vitiligo identifies 70 to 110,000 risk alleles for vitiligo, e.g., 100 to 100,000 risk alleles, 250 to 150,000 risk alleles, 500 to 100,000 risk alleles, 1000 to 50,000 risk alleles, 2000 to 25,000 risk alleles, 3000 to 20,000 risk alleles. 4,000 to 15,000 risk alleles, or 5,000 to 10,000 risk alleles.

Ib. Determination of Individual PRS

In some aspects, the PRS of an individual is represented as the number of SNPs associated with risk for hypothyroidism or vitiligo (“risk alleles”) occurring in the individual as counted over a defined number of sequenced base pairs. In some aspects, the number of sequenced base pairs (bp) is, e.g., at least 50 bp, at least 100 bp, at least 500 bp, at least 1 kbp, at least 10 kbp, at least 50 kbp, at least 100 kbp, at least 500 kbp, at least 1000 kbp, at least 1 Mbp, at least 500 Mbp, or at least 1 Gbp. In other aspects, the sequenced base pairs comprise the whole genome sequence (WGS) of an individual. Methods for WGS include, but are not limited to, the Illumina X10 HiSeq platform. In some aspects, WGS data is generated to an average read depth of at least 2×, at least 5×, at least 10×, at least 15×, at least 20×, at least 25×, at least 30×, at least 35×, at least 40×, at least 45×, at least 50×, or at least 100×coverage. Reads may be mapped to a reference genome, e.g., a human reference genome, e.g., hg38/GRCh38 (GCA_000001405.15). See, for example, Van der Auwera et al., Curr Protoc Bioinformatics, 11: 11.10.1-11.10.33, 2013; McKenna et al., Genome Res., 20: 1297-1303, 2010; and DePristo et al., Nat. Genet., 43: 491-498, 2011.

PRSs may be assessed in one or more samples from an individual. A sample may be a tissue sample (e.g., a tissue biopsy), a cell sample, a whole blood sample, a buccal swab, a plasma sample, a serum sample, or a combination thereof. In some aspects, the sample contains germline DNA. In some aspects, the sample is a formalin-fixed and paraffin-embedded (FFPE) sample, an archival sample, a fresh sample, or a frozen sample.

In some aspects, a PRS for hypothyroidism may be determined for a sample from an individual. In some aspects, the PRS identifies 0, 1 or more, 5 or more, 10 or more, 15 or more, 20 or more, 25 or more, 30 or more, 40 or more, 50 or more, 60 or more, 70 or more, 80 or more, 90 or more, 100 or more, 150 or more, 200 or more, 300 or more, 400 or more, 500 or more, 1000 or more, 2000 or more, 3000 or more, 4000 or more, 5000 or more, 10,000 or more, 50,000 or more, 100,000 or more, 200,000 or more, or 500,000 or more risk alleles for hypothyroidism in the sample from the individual. In some aspects, the PRS score for hypothyroidism of the individual is higher than 0%, higher than 10%, higher than 20%, higher than 30%, higher than 40%, higher than 50%, higher than 60%, higher than 70%, higher than 80%, higher than 90%, or higher than 100% of PRS scores for hypothyroidism for individuals in a reference population.

In one aspect, the PRS of an individual for hypothyroidism is represented as the number of SNPs associated with risk for hypothyroidism counted in a WGS sample, wherein the sample is a blood sample or a buccal swab.

In some aspects, a PRS for vitiligo may be determined for a sample from an individual. In some aspects, the PRS identifies 0, 1 or more, 5 or more, 10 or more, 15 or more, 20 or more, 25 or more, 30 or more, 40 or more, 50 or more, 60 or more, 70 or more, 80 or more, 90 or more, 100 or more, 150 or more, 200 or more, 300 or more, 400 or more, 500 or more, 1000 or more, 2000 or more, 3000 or more, 4000 or more, 5000 or more, 10,000 or more, 50,000 or more, 100,000 or more, 200,000 or more, or 500,000 or more risk alleles for vitiligo in the sample from the individual. In some aspects, the PRS score for vitiligo of the individual is higher than 0%, higher than 10%, higher than 20%, higher than 30%, higher than 40%, higher than 50%, higher than 60%, higher than 70%, higher than 80%, higher than 90%, or higher than 100% of PRS scores for vitiligo for individuals in a reference population.

In one aspect, the PRS of an individual for vitiligo is represented as the number of SNPs associated with risk for vitiligo counted in a WGS sample, wherein the sample is a blood sample or a buccal swab.

In some aspects, PRSs may be determined for an individual for at least two autoimmune diseases. In some aspects, a PRS for hypothyroidism and a PRS for vitiligo are determined for an individual.

In some aspects, the PRS for hypothyroidism or vitiligo of the sample from the individual or the PRS for hypothyroidism or vitiligo of a sample from an individual in the reference population is calculated using the equation:

$$\hat{S} = {\sum }_{i = 1}^{M} {β}_{i} \cdot {G}_{i}$$

wherein Ŝ is the PRS for hypothyroidism; M is the number of risk alleles selected from independent genetic signals in a genome-wide association study (GWAS) for hypothyroidism or vitiligo; i represents the index of a given SNP; βᵢ is the log odds ratio or conditionally independent odds ratio of the ith SNP; and Gᵢ={0,1,2} is the number of copies of the SNP in the sample from the individual.

In some aspects, the risk alleles are selected from Table 7 and/or Table 8.

In some embodiments, M is the number of independent signals in the GWAS after fine mapping, βᵢ corresponds to the conditional effect size for the variant with the highest PPA for ith signal, and Gᵢ={0,1,2} corresponds to the number of copies of the risk allele.

Ic. Reference Populations

In some aspects, the PRS of an individual for hypothyroidism or vitiligo is compared to PRSs in a reference population. In some aspects, the reference population is a population of individuals having a cancer, the population of individuals consisting of a first subset of individuals who have been treated with an immune checkpoint inhibitor therapy, e.g., an immune checkpoint inhibitor described in Section IIIB herein, e.g., a PD-L1 axis binding antagonist, and a second subset of individuals who have been treated with a non-immune checkpoint inhibitor therapy e.g., a non-immune checkpoint inhibitor therapy described in Section lID herein, e.g., a chemotherapy, wherein the non-immune checkpoint inhibitor therapy does not comprise an immune checkpoint inhibitor. In other aspects, the reference population is the GWAS population.

In some aspects, the reference population may be used to determine a hypothyroidism reference PRS and/or a vitiligo reference PRS. The reference population may be used to determine one or both of a hypothyroidism reference PRS and a vitiligo reference PRS. In some aspects, the reference is a PRS value that significantly separates each of the first subset and the second subsets of individuals based on a significant difference in responsiveness to treatment with the immune checkpoint inhibitor therapy relative to responsiveness to treatment with the non-immune checkpoint inhibitor therapy. The difference in responsiveness to treatment may be, for example, a difference in overall survival (OS) or progression-free survival (PFS).

In some aspects, the hypothyroidism reference PRS is defined as, e.g., the 0^{th} percentile, 1^{st} percentile, 2^{nd} percentile, 3^{rd} percentile, 4^{th} percentile, 5^{th} percentile, 6^{th} percentile, 7^{th} percentile, 8^{th} percentile, 9^{th} percentile, 10^{th} percentile, 11^{th} percentile, 12^{th} percentile, 13^{th} percentile, 14^{th} percentile, 15^{th} percentile, 16^{th} percentile, 17^{th} percentile, 18^{th} percentile, 19^{th} percentile, 20^{th} percentile, 21^{st} percentile, 22^{nd} percentile, 23^{rd} percentile, 24^{th} percentile, 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of PRSs for hypothyroidism in the reference population.

In some aspects, the vitiligo reference PRS is defined as the 25^{th} percentile of PRSs for vitiligo in the reference population. In some aspects, the vitiligo reference PRS is defined as the 50^{th} percentile of PRSs for vitiligo in the reference population. In some aspects, the vitiligo reference PRS is defined as the median of PRSs for vitiligo in the reference population. In some aspects, the vitiligo reference PRS is defined as the 75^{th} percentile of PRSs for vitiligo in the reference population.

In some aspects, the vitiligo reference PRS is defined as, e.g., the 0^{th} percentile, 1^{st} percentile, 2^{nd} percentile, 3^{rd} percentile, 4^{th} percentile, 5^{th} percentile, 6^{th} percentile, 7^{th} percentile, 8^{th} percentile, 9^{th} percentile, 10^{th} percentile, 11^{th} percentile, 12^{th} percentile, 13^{th} percentile, 14^{th} percentile, 15^{th} percentile, 16^{th} percentile, 17^{th} percentile, 18^{th} percentile, 19^{th} percentile, 20^{th} percentile, 21^{st} percentile, 22^{nd} percentile, 23^{rd} percentile, 24^{th} percentile, 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of PRSs for vitiligo in the reference population.

In some aspects, the vitiligo reference PRS is defined as the 25^{th} percentile of PRSs for vitiligo in the reference population. In some aspects, the vitiligo reference PRS is defined as the 50^{th} percentile of PRSs for vitiligo in the reference population. In some aspects, the vitiligo reference PRS is defined as the median of PRSs for vitiligo in the reference population. In some aspects, the vitiligo reference PRS is defined as the 75^{th} percentile of PRSs for vitiligo in the reference population.

B. Methods of Identifying Patients Likely to Experience Endocrine irAEs

In some aspects, the invention features a method of identifying an individual having a cancer who has an increased likelihood of experiencing treatment-induced thyroid dysfunction during treatment comprising an immune checkpoint inhibitor, the method comprising determining a polygenic risk score (PRS) for one or both of hypothyroidism and vitiligo from a sample from the individual, wherein (a) a PRS for hypothyroidism that is above a hypothyroidism reference PRS and/or (b) a PRS for vitiligo that is above a vitiligo reference PRS (e.g., a reference PRS as defined in Section IIA) identifies the individual as one who may have an increased likelihood of experiencing treatment-induced thyroid dysfunction during treatment comprising an immune checkpoint inhibitor (e.g., a PD-L1 axis binding antagonist (e.g., a PD-L1 binding antagonist, e.g., atezolizumab)). In some aspects, the thyroid dysfunction is hypothyroidism. In other aspects, the thyroid dysfunction is hyperthyroidism. In still other aspects, the thyroid dysfunction comprises both hypothyroidism and hyperthyroidism, e.g., comprises hyperthyroidism followed by hypothyroidism or hypothyroidism followed by hyperthyroidism.

In some aspects, the PRS of the individual for hypothyroidism is greater than 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of PRSs for hypothyroidism in the reference population.

In some aspects, the hypothyroidism reference PRS is defined as the median of PRSs for hypothyroidism in the reference population, and the PRS for hypothyroidism of the individual is greater than the median of PRSs for hypothyroidism in the reference population.

In other aspects, the hypothyroidism reference PRS is defined as the median of PRSs for hypothyroidism in the reference population, and the PRS for hypothyroidism of the individual is less than the median of PRSs for hypothyroidism in the reference population.

In some aspects, the PRS of the individual for vitiligo is greater than 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of PRSs for vitiligo in the reference population.

In some aspects, the vitiligo reference PRS is defined as the median of PRSs for vitiligo in the reference population, and the PRS for vitiligo of the individual is greater than the median of PRSs for vitiligo in the reference population.

In other aspects, the hypothyroidism reference PRS is defined as the median of PRSs for vitiligo in the reference population, and the PRS for vitiligo of the individual is less than the median of PRSs for vitiligo in the reference population.

In some aspects, the cancer is metastatic urothelial carcinoma, non-squamous non-small cell lung cancer (NSCLC), small cell lung cancer (SCLC), renal cell carcinoma (RCC), or triple negative breast cancer (TNBC). In some aspects, the treatment comprising an immune checkpoint inhibitor is second-line (2L) treatment of metastatic urothelial carcinoma, first-line (1 L) treatment of NSCLC, or first-line (1 L) treatment of squamous NSCLC. In some aspects, the method comprises administering an immune checkpoint inhibitor as 2L treatment of metastatic urothelial carcinoma, 1 L treatment of NSCLC, or 1 L treatment of squamous NSCLC.

i. TSH Levels and Gender

In some aspects, the method further comprises assessing one or more properties that are positively associated with the predictive capacity of a PRS for hypothyroidism from a sample from the individual before administration of a treatment including an immune checkpoint inhibitor (e.g., a PD-L1 axis binding antagonist (e.g., a PD-L1 binding antagonist, e.g., atezolizumab)).

In some aspects, the property is a level of thyroid-stimulating hormone (TSH) that is above a TSH reference level. In some aspects, the TSH reference level is a pre-assigned TSH level. In some aspects, the TSH reference level is the median TSH level in the reference population.

In some aspects, the TSH reference level is defined as, e.g., the 0^{th} percentile, 1^{st} percentile, 2^{nd} percentile, 3^{rd} percentile, 4^{th} percentile, 5^{th} percentile, 6^{th} percentile, 7^{th} percentile, 8^{th} percentile, 9^{th} percentile, 10^{th} percentile, 11^{th} percentile, 12^{th} percentile, 13^{th} percentile, 14^{th} percentile, 15^{th} percentile, 16^{th} percentile, 17^{th} percentile, 18^{th} percentile, 19^{th} percentile, 20^{th} percentile, 21^{st} percentile, 22^{nd} percentile, 23^{rd} percentile, 24^{th} percentile, 25^{th} percentile, 26^{th} percentile, 27^{th} percentile, 28^{th} percentile, 29^{th} percentile, 30^{th} percentile, 31^{st} percentile, 32^{nd} percentile, 33^{rd} percentile, 34^{th} percentile, 35^{th} percentile, 36^{th} percentile, 37^{th} percentile, 38^{th} percentile, 39^{th} percentile, 40^{th} percentile, 41^{st} percentile, 42^{nd} percentile, 43^{rd} percentile, 44^{th} percentile, 45^{th} percentile, 46^{th} percentile, 47^{th} percentile, 48^{th} percentile, 49^{th} percentile, 50^{th} percentile, 51^{st} percentile, 52^{nd} percentile, 53^{rd} percentile, 54^{th} percentile, 55^{th} percentile, 56^{th} percentile, 57^{th} percentile, 58^{th} percentile, 59^{th} percentile, 60^{th} percentile, 61^{st} percentile, 62^{nd} percentile, 63^{rd} percentile, 64^{th} percentile, 65^{th} percentile, 66^{th} percentile, 67^{th} percentile, 68^{th} percentile, 69^{th} percentile, 70^{th} percentile, 71^{st} percentile, 72^{nd} percentile, 73^{rd} percentile, 74^{th} percentile, 75^{th} percentile, 76^{th} percentile, 77^{th} percentile, 78^{th} percentile, 79^{th} percentile, 80^{th} percentile, 81^{st} percentile, 82^{nd} percentile, 83^{rd} percentile, 84^{th} percentile, 85^{th} percentile, 86^{th} percentile, 87^{th} percentile, 88^{th} percentile, 89^{th} percentile, 90^{th} percentile, 91^{st} percentile, 92^{nd} percentile, 93^{rd} percentile, 94^{th} percentile, 95^{th} percentile, 96^{th} percentile, 97^{th} percentile, 98^{th} percentile, or 99^{th} percentile of TSH levels in the reference population.

In some aspects, the TSH reference level is defined as the 25^{th} percentile of TSH levels in the reference population. In some aspects, the TSH reference level is defined as the 50^{th} percentile of TSH levels in the reference population. In some aspects, the TSH reference level is defined as the median of TSH levels in the reference population. In some aspects, the TSH reference level is defined as the 75^{th} percentile of TSH levels in the reference population.

In some aspects, the property that is positively associated with the predictive capacity of a PRS for hypothyroidism is female sex.

In some aspects, the individual is female and has a level of TSH that is above a TSH reference level.

C. Methods of Identifying Patients Likely to Benefit from Treatment with an Immune Checkpoint Inhibitor

In some aspects, a hypothyroidism PRS score of an individual is used in determining whether to treat a patient with an immune checkpoint inhibitor, e.g., an immune checkpoint inhibitor described in Section IIIB herein (e.g., a PD-L1 axis binding antagonist (e.g., a PD-L1 binding antagonist, e.g., atezolizumab) and/or an anti-TIGIT antagonist antibody (e.g., tiragolumab)).

In some aspects, the invention features a method of identifying an individual having a breast cancer (e.g., a triple-negative breast cancer (TNBC)) who may benefit from a treatment comprising an immune checkpoint inhibitor, the method comprising determining a polygenic risk score (PRS) for hypothyroidism from a sample from the individual, wherein a PRS for hypothyroidism that is above a hypothyroidism reference PRS (e.g., a hypothyroidism reference PRS defined in Section IIA) identifies the individual as one who may receive a benefit from the treatment comprising an immune checkpoint inhibitor.

In some aspects, the invention features a method for selecting a treatment for an individual having a breast cancer (e.g., a TNBC), the method comprising determining a PRS for hypothyroidism from a sample from the individual, wherein a PRS for hypothyroidism that is above a hypothyroidism reference PRS identifies the individual as one who may receive a benefit from a treatment comprising an immune checkpoint inhibitor (e.g., a PD-L1 axis binding antagonist (e.g., a PD-L1 binding antagonist, e.g., atezolizumab)).

In some aspects, the benefit is an increase in overall survival (OS), e.g., an increase in OS of at least 30, 60, 90, 120, 150, 180, 210, 240, 270, 300, 310, 320, or 330 days or an increase in OS of more than 330 days. In some aspects, the increase in OS is an increase of about 30-90 days, 90-150 days, 150-210 days, 210-270 days, or 270-330 days (e.g., about 300-320 days). In some aspects, the increase in OS is an increase of about 320 days.

In some aspects, the PRS of the individual for hypothyroidism is greater than 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of PRSs for hypothyroidism in the reference population.

In some aspects, the hypothyroidism reference PRS is defined as the 50^{th} percentile of PRSs for hypothyroidism in the reference population, and the PRS for hypothyroidism of the individual is greater than 50% of PRSs for hypothyroidism in the reference population.

In other aspects, the hypothyroidism reference PRS is defined as the 50^{th} percentile of PRSs for hypothyroidism in the reference population, and the PRS for hypothyroidism of the individual is less than 50% of PRSs for hypothyroidism in the reference population.

In some aspects, the hypothyroidism reference PRS is defined as the median of PRSs for hypothyroidism in the reference population, and the PRS for hypothyroidism of the individual is greater than the median of PRSs for hypothyroidism in the reference population.

In other aspects, the hypothyroidism reference PRS is defined as the median of PRSs for hypothyroidism in the reference population, and the PRS for hypothyroidism of the individual is less than the median of PRSs for hypothyroidism in the reference population.

In some aspects, the PRS for hypothyroidism determined from the sample is above the hypothyroidism reference PRS and the method further comprises administering to the individual an effective amount of an immune checkpoint inhibitor.

In some aspects, the invention features an immune checkpoint inhibitor for use in treating an individual having a breast cancer (e.g., a TNBC) who has been identified as one who may benefit from a treatment comprising an immune checkpoint inhibitor based on a PRS for hypothyroidism from a sample from the individual that is above a hypothyroidism reference PRS.

In some aspects, the invention features use of an immune checkpoint inhibitor in the manufacture of a medicament for treating an individual having a breast cancer (e.g., a TNBC) who has been identified as one who may benefit from a treatment comprising an immune checkpoint inhibitor based on a PRS for hypothyroidism from a sample from the individual that is above a hypothyroidism reference PRS.

iv. Methods of Assessing Tumor-Associated Factors

The presence or absence of one or more factors in the tumor or the tumor microenvironment (“tumor-associated factors”) may be associated with the efficacy of immune checkpoint inhibitor therapy. These factors include high immune cell (IC) staining of PD-L1. In some aspects, IC staining of PD-L1 is measured in one or more tumor samples for an individual for which a PRS for hypothyroidism or a PRS for vitiligo is also measured. Analysis of IC staining of PD-L1 can occur prior to, simultaneously, and/or following determination of the PRS for hypothyroidism or PRS for vitiligo of the individual.

Tumor-associated factors (e.g., IC staining of PD-L1) may be assessed in one or more samples from an individual. A sample may be a tissue sample, a tissue biopsy, a cell sample, a whole blood sample, a plasma sample, a serum sample, or a combination thereof. In some aspects, the tissue sample is a tumor tissue sample. In some aspects, the tumor tissue sample comprises tumor cells, tumor-infiltrating immune cells, stromal cells, or a combination thereof. The tumor tissue sample may be assessed to confirm the presence of tumor cells and/or the proportion of tumor cells in the sample, e.g., by hematoxylin and eosin (H&E) staining of slides and subsequent observation. The sample may contain, e.g., at least 10% tumor cells. In some aspects, the tumor tissue sample is a formalin-fixed and paraffin-embedded (FFPE) sample, an archival sample, a fresh sample, or a frozen sample.

iii(a). IC Staining of PD-L1

In some aspects, the level of immune cell (IC) staining (e.g., by immunohistochemistry (IHC)) for PD-L1 of a sample from the tumor of the individual is quantified. IC staining may be reported as, e.g., ICO (no evidence of immune cell staining of PD-L1) or as IC1, IC2, or IC3, designating increasing levels of immune cell PD-L1 staining as defined in Powles et al., Lancet, 391: 748-757, 2018. Likewise, the level of tumor cell (TC) staining (e.g., by immunohistochemistry) for PD-L1 of a sample from the tumor of the individual may be quantified. TC staining may be reported as, e.g., TCO (no evidence of immune cell staining of PD-L1) or as TC1, TC2, or TC3, designating increasing levels of tumor cell PD-L1 staining, as defined in Table 2. Low IC staining of PD-L1 may be defined as, e.g., ICO or ICO and IC1. Staining may be performed using a diagnostic anti-human PD-L1 monoclonal antibody, e.g., 22C3, SP142, SP263, or 28-8. In some aspects, the diagnostic antibody is SP142. SP142 is described in US Patent Application Publication No. 2018/0022809. In some aspects, the protocol for staining is the VENTANA PD-L1 SP142 immunohistochemistry (IHC) assay.

The amino acid sequence of the heavy chain variable region of SP142 is the following:

```
(SEQ ID NO: 32)
QSLEESGGRLVKPDETLTITCTVSGIDLSSNGLTWVRQAPGEG
                                                          HVR-H1
LEWIGTINKDASAYYASWAKGRLTI
                  HVR-H2
SKPSSTKVDLKITSPTTEDTATYFCGRIAFKTGTSIWGPGTLVTVSS.
                                                          HVR-H3
```

The amino acid sequence of the light chain variable region of SP142 is the following:

```
(SEQ ID NO: 33)
AIVMTQTPSPVSAAVGGTVTINCQASESVYSNNYLSWFQ
                                                    HVR-L1
QKPGQPPKLLIYLASTLASGVPSRFKGS
                          HVR-L2
GSGTQFTLTISGVQCDDAATYYCIGGKSSSTDGNAFGGGTEVVVR.
                                                    HVR-L3
```

In some aspects, detectable PD-L1 staining is present in tumor-infiltrating immune cells covering <1% of the tumor area.

In some aspects, detectable PD-L1 staining is present in tumor-infiltrating immune cells covering ≥5% of the tumor area.

In some aspects, detectable PD-L1 staining is present in tumor-infiltrating immune cells covering ≥50% of the tumor area.

TABLE 2
PD-L1 scoring criteria on TC and IC using the SP142 assay

| PD-L1 TC Scoring | | PD-L1 IC Scoring | |
|---|---|---|---|
| ^{a}TC Score | % of PD-L1-Expressing TC | ^{b}IC Score | % of PD-L1-Expressing IC |
| TC3 | Presence of discernible PD-L1 | IC3 | Presence of discernible PD-L1 |
| | staining of any intensity in ≥50% | | staining of any intensity in |
| | of tumor cells | | tumor-infiltrating immune cells |
| | | | covering ≥10% of tumor area |
| | | | occupied by tumor cells, |
| | | | associated intratumoral stroma, |
| | | | and contiguous peri-tumoral |
| | | | desmoplastic stroma |
| TC2 | Presence of discernible PD-L1 | IC2 | Presence of discernible PD-L1 |
| | staining of any intensity in ≥5% | | staining of any intensity in tumor- |
| | to <50% of tumor cells | | infiltrating immune cells |
| | | | covering ≥5% to <10% of tumor |
| | | | area occupied by tumor cells, |
| | | | associated intratumoral |
| | | | stroma, and contiguous |
| | | | peri-tumoral desmoplastic stroma |
| TC1 | Presence of discernible PD-L1 | IC1 | Presence of discernible PD-L1 |
| | staining of any intensity in ≥1% | | staining of any intensity in tumor- |
| | to <5% of tumor cells | | infiltrating immune cells |
| | | | covering ≥1% to <5% of tumor |
| | | | area occupied by tumor cells, |
| | | | associated intratumoral |
| | | | stroma, and contiguous |
| | | | peri-tumoral desmoplastic stroma |
| TC0 | Absence of any discernible PD-L1 | IC0 | Absence of any discernible PD-L1 |
| | staining | | staining |
| | OR | | OR |
| | Presence of discernible PD-L1 | | Presence of discernible PD-L1 |
| | staining of any intensity in <1% | | staining of any intensity in tumor- |
| | of tumor cells | | infiltrating immune cells |
| | | | covering <1% of tumor area |
| | | | occupied by tumor cells, |
| | | | associated intratumoral |
| | | | stroma, and contiguous |
| | | | peri-tumoral desmoplastic stroma |

^{a}TC scored as percentage of tumor cells; ^{b}IC scored as percentage of tumor area.

### III. Methods of Treatment

In some aspects, the invention features a method of treating an individual having a cancer, the method comprising (a) determining a PRS for one or both of hypothyroidism and vitiligo from a sample from the individual, wherein the PRS for hypothyroidism is above a hypothyroidism reference PRS and/or the PRS for vitiligo is above a vitiligo reference PRS; (b) administering an effective amount of an immune checkpoint inhibitor to the individual (e.g., a PD-L1 axis binding antagonist (e.g., a PD-L1 binding antagonist, e.g., atezolizumab)) and/or an anti-TIGIT antagonist antibody (e.g., tiragolumab)); and (c) monitoring the individual for symptoms of thyroid dysfunction. In some aspects, the thyroid dysfunction is hypothyroidism. In other aspects, the thyroid dysfunction is hyperthyroidism. In still other aspects, the thyroid dysfunction comprises both hypothyroidism and hyperthyroidism, e.g., comprises hyperthyroidism followed by hypothyroidism or hypothyroidism followed by hyperthyroidism.

In some aspects, the individual is monitored for symptoms of thyroid dysfunction prior to and periodically during treatment with the immune checkpoint inhibitor. Hormone replacement therapy or medical management of hyperthyroidism may be initiated as clinically indicated. In some aspects, treatment with the immune checkpoint inhibitor is continued in the case of hypothyroidism and interrupted in the case of hyperthyroidism based on the severity (e.g., interrupted in the case of severe hyperthyroidism).

In some aspects, the invention features a method of treating an individual having a breast cancer (e.g., a TNBC), the method comprising (a) determining a PRS for hypothyroidism from a sample from the individual, wherein the PRS for hypothyroidism from the sample is above a hypothyroidism reference PRS; and (b) administering an effective amount of an immune checkpoint inhibitor (e.g., a PD-L1 axis binding antagonist (e.g., a PD-L1 binding antagonist, e.g., atezolizumab)) to the individual.

In some aspects, the invention features a method of treating an individual having a breast cancer (e.g., a TNBC), the method comprising administering an immune checkpoint inhibitor (e.g., a PD-L1 axis binding antagonist (e.g., a PD-L1 binding antagonist, e.g., atezolizumab)) to the individual who has been determined to have a PRS for hypothyroidism that is above a hypothyroidism reference PRS.

In some aspects, the benefit is an increase in overall survival (OS), e.g., an increase in OS of at least 30, 60, 90, 120, 150, 180, 210, 240, 270, 300, 310, 320, or 330 days or an increase in OS of more than 330 days. In some aspects, the increase in OS is an increase of about 30-90 days, 90-150 days, 150-210 days, 210-270 days, or 270-330 days (e.g., about 300-320 days). In some aspects, the increase in OS is an increase of about 320 days.

### A. Cancers

In some aspects, an immune checkpoint inhibitor is used to treat or delay progression of a cancer in a subject in need thereof. In some aspects, the subject is a human. The cancer may be a solid tumor cancer or a non-solid tumor cancer. Solid cancer tumors include, but are not limited to a breast cancer, a bladder cancer, a lung cancer, a kidney cancer, a melanoma, a colorectal cancer, a head and neck cancer, an ovarian cancer, a pancreatic cancer, or a prostate cancer, or metastatic forms thereof. In some aspects, the cancer is a bladder cancer. In some aspects, the cancer is a breast cancer. Further aspects of breast cancer include a triple-negative breast cancer (TNBC). Other aspects of breast cancer include a hormone receptor-positive (HR+) breast cancer, e.g., an estrogen receptor-positive (ER+) breast cancer, a progesterone receptor-positive (PR+) breast cancer, or an ER+/PR+ breast cancer. Yet other aspects of breast cancer include a HER2-positive (HER2+) breast cancer. In some aspects, the breast cancer is an early breast cancer. In some aspects, the cancer is a bladder cancer. Further aspects of bladder cancer include urothelial carcinoma (UC), muscle invasive bladder cancer (MIBC), and non-muscle invasive bladder cancer (NMIBC). In some aspects, the bladder cancer is a metastatic urothelial carcinoma (mUC). In some aspects, the mUC is a second line (2L) mUC. In some aspects, the cancer is a lung cancer. Further aspects of lung cancer include an epidermal growth factor receptor-positive (EGFR+) lung cancer. Other aspects of lung cancer include an epidermal growth factor receptor-negative (EGFR−) lung cancer. Yet other aspects of lung cancer include a non-small cell lung cancer (NSCLC), e.g., a squamous lung cancer or a non-squamous lung cancer. In some aspects, the NSCLC is a 1 L non-squamous NSCLC or a 1 L squamous NSCLC. Other aspects of lung cancer include a small cell lung cancer (SCLC). In some aspects, the cancer is a kidney cancer. Further aspects of kidney cancer include a renal cell carcinoma (RCC). In some aspects, the cancer is a head and neck cancer. Further aspects of head and neck cancer include a squamous cell carcinoma of the head & neck (SCCHN). In some aspects, the cancer is a liver cancer. Further aspects of liver cancer include a hepatocellular carcinoma. In some aspects, the cancer is a prostate cancer. Further aspects of prostate cancer include a castration-resistant prostate cancer (CRPC). In some aspects, the cancer is a metastatic form of a solid tumor. In some aspects, the metastatic form of a solid tumor is a metastatic form of a melanoma, a breast cancer, a colorectal cancer, a lung cancer, a head and neck cancer, a bladder cancer, a kidney cancer, an ovarian cancer, a pancreatic cancer, or a prostate cancer. In some aspects, the cancer is a non-solid tumor cancer. Non-solid tumor cancers include, but are not limited to, a B-cell lymphoma. Further aspects of B-cell lymphoma include, e.g., a chronic lymphocytic leukemia (CLL), a diffuse large B-cell lymphoma (DLBCL), a follicular lymphoma, myelodysplastic syndrome (MDS), a non-Hodgkin lymphoma (NHL), an acute lymphoblastic leukemia (ALL), a multiple myeloma, an acute myeloid leukemia (AML), or a mycosis fungoides (MF).

In some aspects, the cancer is metastatic urothelial carcinoma, non-squamous non-small cell lung cancer (NSCLC), small cell lung cancer (SCLC), renal cell carcinoma (RCC), or triple negative breast cancer (TNBC). In some aspects, the treatment comprising an immune checkpoint inhibitor is second-line (2L) treatment of metastatic urothelial carcinoma, first-line (1 L) treatment of NSCLC, or first-line (1 L) treatment of squamous NSCLC. In some aspects, the method comprises administering an immune checkpoint inhibitor as 2L treatment of metastatic urothelial carcinoma, 1 L treatment of NSCLC, or 1 L treatment of squamous NSCLC.

### B. Immune Checkpoint Inhibitors

i. PD-L1 Axis Binding Antagonists

In some aspects, an immune checkpoint inhibitor, for example, a PD-L1 axis binding antagonist, includes a PD-1 binding antagonist, a PD-L1 binding antagonist, and a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1LG1,” “CD274,” “B7-H,” and “PDL1.” An exemplary human PD-L1 is shown in UniProtKB/Swiss-Prot Accession No.Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some aspects, the PD-1 binding antagonist is a molecule that inhibits the binding of PD-1 to its ligand binding partners. In a specific aspect the PD-1 ligand binding partners are PD-L1 and/or PD-L2. In another instance, a PD-L1 binding antagonist is a molecule that inhibits the binding of PD-L1 to its binding ligands. In a specific aspect, PD-L1 binding partners are PD-1 and/or B7-1. In another instance, the PD-L2 binding antagonist is a molecule that inhibits the binding of PD-L2 to its ligand binding partners. In a specific aspect, the PD-L2 binding ligand partner is PD-1. The antagonist may be an antibody, an antigen binding fragment thereof, an immunoadhesin, a fusion protein, or oligopeptide.

In some aspects, the PD-1 binding antagonist is an anti-PD-1 antibody (e.g., a human antibody, a humanized antibody, or a chimeric antibody), for example, as described below. In some aspects, the anti-PD-1 antibody is selected from the group consisting of MDX-1106 (nivolumab), MK-3475 (pembrolizumab), MEDI-0680 (AMP-514), PDR001, REGN2810, and BGB-108. MDX-1106, also known as MDX-1106-04, ONO-4538, BMS-936558, or nivolumab, is an anti-PD-1 antibody described in WO2006/121168. MK-3475, also known as pembrolizumab or lambrolizumab, is an anti-PD-1 antibody described in WO 2009/114335. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some instances, the PD-1 binding antagonist is AMP-224. AMP-224, also known as B7-DCIg, is a PD-L2-Fc fusion soluble receptor described in WO 2010/027827 and WO 2011/066342.

In some aspects, the anti-PD-1 antibody is MDX-1106. Alternative names for “MDX-1106” include MDX-1106-04, ONO-4538, BMS-936558, and nivolumab. In some aspects, the anti-PD-1 antibody is nivolumab (CAS Registry Number: 946414-94-4). In a still further aspect, provided is an isolated anti-PD-1 antibody comprising a heavy chain variable region comprising the heavy chain variable region amino acid sequence from SEQ ID NO: 1 and/or a light chain variable region comprising the light chain variable region amino acid sequence from SEQ ID NO: 2. In a still further aspect, provided is an isolated anti-PD-1 antibody comprising a heavy chain and/or a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 1)
QVQLVESGGGVVQPGRSLRLDCKASGITFSNSGMHWVRQAP
GKGLEWVAVIWYDGSKRYYADSVKGRFTISRDNSKNTLFL
QMNSLRAEDTAVYYCATNDDYWGQGTLVTVSSASTKGPSV
FPLAPCSRSTSESTAALGCLVKDYFPEPVTVSWNSGALTS
GVHTFPAVLQSSGLYSLSSVVTVPSSSLGTKTYTCNVDHK
PSNTKVDKRVESKYGPPCPPCPAPEFLGGPSVFLFPPKPK
DTLMISRTPEVTCVVVDVSQEDPEVQFNWYVDGVEVHNAK
TKPREEQFNSTYRVVSVLTVLHQDWLNGKEYKCKVSNKGL
PSSIEKTISKAKGQPREPQVYTLPPSQEEMTKNQVSLTCL
VKGFYPSDIAVEWESNGQPENNYKTTPPVLDSDGSFFLYS
RLTVDKSRWQEGNVFSCSVMHEALHNHYTQKSLSLSLGK,

and
(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 2)
EIVLTQSPATLSLSPGERATLSCRASQSVSSYLAWYQQKPGQAP
RLLIYDASNRATGIPARFSGSGSGTDFTLTISSLEPEDFAVYYC
QQSSNWPRTFGQGTKVEIKRTVAAPSVFIFPPSDEQLKSGTASV
VCLLNNFYPREAKVQWKVDNALQSGNSQESVTEQDSKDSTYSLS
STLTLSKADYEKHKVYACEVTHQGLSSPVTKSFNRGEC.

In some aspects, the PD-L1 axis binding antagonist is a PD-L2 binding antagonist. In some aspects, the PD-L2 binding antagonist is an anti-PD-L2 antibody (e.g., a human antibody, a humanized antibody, or a chimeric antibody). In some aspects, the PD-L2 binding antagonist is an immunoadhesin.

In some aspects, the PD-L1 binding antagonist is an anti-PD-L1 antibody, for example, as described below. In some aspects, the anti-PD-L1 antibody is capable of inhibiting binding between PD-L1 and PD-1 and/or between PD-L1 and B7-1. In some aspects, the anti-PD-L1 antibody is a monoclonal antibody. In some aspects, the anti-PD-L1 antibody is an antibody fragment selected from the group consisting of Fab, Fab′-SH, Fv, scFv, and (Fab′)₂ fragments. In some aspects, the anti-PD-L1 antibody is a humanized antibody. In some aspects, the anti-PD-L1 antibody is a human antibody. In some aspects, the anti-PD-L1 antibody is selected from the group consisting of atezolizumab, MDX-1105, and MEDI4736 (durvalumab), and MSB0010718C (avelumab). MDX-1105, also known as BMS-936559, is an anti-PD-L1 antibody described in WO2007/005874. MEDI4736 (durvalumab) is an anti-PD-L1 monoclonal antibody described in WO2011/066389 and US2013/034559. Examples of anti-PD-L1 antibodies useful for the methods of this invention, and methods for making thereof are described in PCT patent application WO 2010/077634, WO 2007/005874, WO 2011/066389, U.S. Pat. No. 8,217,149, and US 2013/034559, which are incorporated herein by reference.

Anti-PD-L1 antibodies described in WO 2010/077634 A1 and U.S. Pat. No. 8,217,149 may be used in the methods described herein. In some aspects, the anti-PD-L1 antibody comprises a heavy chain variable region sequence of SEQ ID NO: 3 and/or a light chain variable region sequence of SEQ ID NO: 4. In a still further aspect, provided is an isolated anti-PD-L1 antibody comprising a heavy chain variable region and/or a light chain variable region sequence, wherein:
(a) the heavy chain variable region sequence has at least 85%, at least 90%, at least 91%, at least 92%, at least 93%, at least 94%, at least 95%, at least 96%, at least 97%, at least 98%, at least 99% or 100% sequence identity to the heavy chain variable region sequence:

(SEQ ID NO: 3)
EVOLVESGGGLVQPGGSLRLSCAASGFTFSDSWIHWVRQAPGKG
LEWVAWISPYGGSTYYADSVKGRFTISADTSKNTAYLQMNSLRA
EDTAVYYCARRHWPGGFDYWGQGTLVTVSS,

and
(b) the light chain variable region sequence has at least 85%, at least 90%, at least 91%, at least 92%, at least 93%, at least 94%, at least 95%, at least 96%, at least 97%, at least 98%, at least 99% or 100% sequence identity to the light chain variable region sequence:

(SEQ ID NO: 4)
DIQMTQSPSSLSASVGDRVTITCRASQDVSTAVAWYQQ
KPGKAPKLLIYSASFLYSGVPSRFSGSGSGTDFTLTIS
SLQPEDFATYYCQQYLYHPATFGQGTKVEIKR.

In one aspect, the anti-PD-L1 antibody comprises a heavy chain variable region comprising an HVR-H1, HVR-H2 and HVR-H3 sequence, wherein:

(a) the HVR-H1 sequence is
(SEQ ID NO: 5)
GFTFSX₁SWIH;
(b) the HVR-H2 sequence is
(SEQ ID NO: 6)
AWIX₂PYGGSX₃YYADSVKG;
(c) the HVR-H3 sequence is
(SEQ ID NO: 7)
RHWPGGFDY;

further wherein: X₁ is D or G; X₂ is S or L; X₃ is T or S. In one specific aspect, X₁ is D; X₂ is S and X₃ is T. In another aspect, the polypeptide further comprises variable region heavy chain framework sequences juxtaposed between the HVRs according to the formula: (FR-H1)-(HVR-H1)-(FR-H2)-(HVR-H2)-(FR-H3)-(HVR-H3)-(FR-H4). In yet another aspect, the framework sequences are derived from human consensus framework sequences. In a further aspect, the framework sequences are VH subgroup III consensus framework. In a still further aspect, at least one of the framework sequences is the following:

FR-H1 is
(SEQ ID NO: 8)
EVQLVESGGGLVQPGGSLRLSCAAS
FR-H2 is
(SEQ ID NO: 9)
WRQAPGKGLEWV
FR-H3 is
(SEQ ID NO: 10)
RFTISADTSKNTAYLQMNSLRAEDTAVYYCAR
FR-H4 is
(SEQ ID NO: 11)
WGQGTLVTVSS.

In a still further aspect, the heavy chain polypeptide is further combined with a variable region light chain comprising an HVR-L1, HVR-L2 and HVR-L3, wherein:

(a) the HVR-L1 sequence is
(SEQ ID NO: 12)
RASQX₄X₅X₆TX₇X₈A;
(b) the HVR-L2 sequence is
(SEQ ID NO: 13)
SASX₉LX₁₀S;
(c) the HVR-L3 sequence is
(SEQ ID NO: 14)
QQX₁₁X₁₂X₁₃X₁₄PX₁₅T;

wherein: X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T. In a still further aspect, X₄ is D; X₅ is V; X₆ is S; X₇ is A; X₈ is V; Xₛ is F; X₁₀ is Y; X₁₁ is Y; X₁₂ is L; X₁₃ is Y; X₁₄ is H; X₁₅ is A.

In a still further aspect, the light chain further comprises variable region light chain framework sequences juxtaposed between the HVRs according to the formula: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the framework sequences are VL kappa I consensus framework. In a still further aspect, at least one of the framework sequence is the following:

FR-L1 is
(SEQ ID NO: 15)
DIQMTQSPSSLSASVGDRVTITC
FR-L2 is
(SEQ ID NO: 16)
WYQQKPGKAPKLLIY
FR-L3 is
(SEQ ID NO: 17)
GVPSRFSGSGSGTDFTLTISSLQPEDFATYYC
FR-L4 is
(SEQ ID NO: 18)
FGQGTKVEIKR.

In another aspect, provided is an isolated anti-PD-L1 antibody or antigen binding fragment comprising a heavy chain and a light chain variable region sequence, wherein:
(a) the heavy chain comprises an HVR-H1 HVR-H2 and HVR-H3 wherein further:

(i) the HVR-H1 sequence is
(SEQ ID NO: 5)
GFTFSX₁SWIH;
(ii) the HVR-H2 sequence is
(SEQ ID NO: 6)
AWIX₂PYGGSX₃YYADSVKG
(iii) the HVR-H3 sequence is
(SEQ ID NO: 7)
RHWPGGFDY,
and

(b) the light chain comprises an HVR-L1, HVR-L2 and HVR-L3, wherein further:

(i)
the HVR-L1 sequence is
(SEQ ID NO: 14)
RASQX₄X₅X₆TX7X₈A
(ii) the HVR-L2 sequence is
(SEQ ID NO: 12)
SASX₉LX₁₀S;
and
(iii) the HVR-L3 sequence is
(SEQ ID NO: 13)
QQX₁₁X₁₂X₁₃X₁₄PX₁₅T;

wherein: X₁ is D or G; X₂ is S or L; X₃ is T or S; X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, Tori; X₁₅ is A, W, R, P or T. In a specific aspect, X₁ is D; X₂ is S and X₃ is T. In another aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A. In yet another aspect, X₁ is D; X₂ is S and X₃ is T, X₄ is D; X₅ is V; X₆ is S; X₇ is A; X₈ is V; X₉ is F; X₁₀ is Y; X₁₁ is Y; X₁₂ is L; X₁₃ is Y; X₁₄ is Hand X₁₅ is A.

In a further aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs:8, 9, 10, and 11. In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs: 15, 16, 17, and 18.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect the minimal effector function results from an “effector-less Fc mutation” or aglycosylation. In still a further aspect, the effector-less Fc mutation is an N297A or D265A/N297A substitution in the constant region.

In yet another aspect, provided is an anti-PD-L1 antibody comprising a heavy chain and a light chain variable region sequence, wherein:
(a) the heavy chain further comprises an HVR-H1, HVR-H2 and an HVR-H3 sequence having at least 85% sequence identity to GFTFSDSWIH (SEQ ID NO: 19), AWISPYGGSTYYADSVKG (SEQ ID NO: 20) and RHWPGGFDY (SEQ ID NO: 21), respectively, or
(b) the light chain further comprises an HVR-L1, HVR-L2 and an HVR-L3 sequence having at least 85% sequence identity to RASQDVSTAVA (SEQ ID NO: 22), SASFLYS (SEQ ID NO: 23) and QQYLYHPAT (SEQ ID NO: 24), respectively.

In a specific aspect, the sequence identity is 86%, 87%, 88%, 89%, 90%, 91%, 92%, 93%, 94%, 95%, 96%, 97%, 98%, 99% or 100%.

In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs: 8, 9, 10, and 11. In a still further aspect, the light chain framework sequences are derived from a Kabat kappa I, II, II, or IV subgroup sequence. In a still further aspect, the light chain framework sequences are VL kappa I consensus framework. In a still further aspect, one or more of the light chain framework sequences are set forth as SEQ ID NOs: 15, 16, 17, and 18.

In a further aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

FR-H1
(SEQ ID NO: 27)
EVQLVESGGGLVQPGGSLRLSCAASGFTFS
FR-H2
(SEQ ID NO: 28)
WVRQAPGKGLEWVA
FR-H3
(SEQ ID NO: 10)
RFTISADTSKNTAYLQMNSLRAEDTAVYYCAR
FR-H4
(SEQ ID NO: 11)
WGQGTLVTVSS.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

FR-L1
(SEQ ID NO: 15)
DIQMTQSPSSLSASVGDRVTITC
FR-L2
(SEQ ID NO: 16)
WYQQKPGKAPKLLIY
FR-L3
(SEQ ID NO: 17)
GVPSRFSGSGSGTDFTLTISSLQPEDFATYYC
FR-L4
(SEQ ID NO: 26)
FGQGTKVEIK.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect the minimal effector function results from an “effector-less Fc mutation” or aglycosylation. In still a further aspect, the effector-less Fc mutation is an N297A or D265A/N297A substitution in the constant region.

In yet another aspect, provided is an anti-PD-L1 antibody comprising a heavy chain and a light chain variable region sequence, wherein:
(c) the heavy chain further comprises an HVR-H1, HVR-H2 and an HVR-H3 sequence having at least 85% sequence identity to GFTFSDSWIH (SEQ ID NO: 19), AWISPYGGSTYYADSVKG (SEQ ID NO: 20) and RHWPGGFDY (SEQ ID NO: 21), respectively, and/or
(d) the light chain further comprises an HVR-L1, HVR-L2 and an HVR-L3 sequence having at least 85% sequence identity to RASQDVSTAVA (SEQ ID NO: 22), SASFLYS (SEQ ID NO: 23) and QQYLYHPAT (SEQ ID NO: 24), respectively.

In a specific aspect, the sequence identity is 86%, 87%, 88%, 89%, 90%, 91%, 92%, 93%, 94%, 95%, 96%, 97%, 98%, 99% or 100%.

In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs: 8, 9, 10, and WGQGTLVTVSSASTK (SEQ ID NO: 29).

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs: 15, 16, 17, and 18. In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation. In still a further aspect, the effector-less Fc mutation is an N297A or D265A/N297A substitution in the constant region.

In a still further aspect, provided is an isolated anti-PD-L1 antibody comprising a heavy chain and a light chain sequence, wherein:
(a) the heavy chain sequence has at least 85% sequence identity to the heavy chain sequence:

(SEQ ID NO: 30)
EVQLVESGGGLVQPGGSLRLSCAASGFTFSDSWIHWVRQA
PGKGLEWVAWISPYGGSTYYADSVKGRFTISADTSKNTAY
LQMNSLRAEDTAVYYCARRHWPGGFDYWGQGTLVTVSSAS
TKGPSVFPLAPSSKSTSGGTAALGCLVKDYFPEPVTVSWN
SGALTSGVHTFPAVLQSSGLYSLSSVVTVPSSSLGTQTYI
CNVNHKPSNTKVDKKVEPKSCDKTHTCPPCPAPELLGGPS
VFLFPPKPKDTLMISRTPEVTCVVVDVSHEDPEVKFNWYV
DGVEVHNAKTKPREEQYASTYRVVSVLTVLHQDWLNGKEY
KCKVSNKALPAPIEKTISKAKGQPREPQVYTLPPSREEMT
KNQVSLTCLVKGFYPSDIAVEWESNGQPENNYKTTPPVLD
SDGSFFLYSKLTVDKSRWQQGNVFSCSVMHEALHNHYTQK
SLSLSPG,

and/or
(b) the light chain sequences has at least 85% sequence identity to the light chain sequence:

(SEQ ID NO: 31)
DIQMTQSPSSLSASVGDRVTITCRASQDVSTAVAWYQQKP
GKAPKLLIYSASFLYSGVPSRFSGSGSGTDFTLTISSLQPE
DFATYYCQQYLYHPATFGQGTKVEIKRTVAAPSVFIFPPSD
EQLKSGTASVVCLLNNFYPREAKVQWKVDNALQSGNSQESV
TEQDSKDSTYSLSSTLTLSKADYEKHKVYACEVTHQGLSSP
VTKSFNRGEC.

In some aspects, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 31. In some aspects, provided is an isolated anti-PD-L1 antibody comprising a heavy chain and a light chain sequence, wherein the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 30. In some aspects, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 31 and the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 30.

In some aspects, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In any of the aspects herein, the isolated anti-PD-L1 antibody can bind to a human PD-L1, for example a human PD-L1 as shown in UniProtKB/Swiss-Prot Accession No. Q9NZQ7.1, or a variant thereof.

In a still further aspect, provided is an isolated nucleic acid encoding any of the antibodies described herein. In some aspects, the nucleic acid further comprises a vector suitable for expression of the nucleic acid encoding any of the previously described anti-PD-L1 antibodies. In a still further specific aspect, the vector is in a host cell suitable for expression of the nucleic acid. In a still further specific aspect, the host cell is a eukaryotic cell or a prokaryotic cell. In a still further specific aspect, the eukaryotic cell is a mammalian cell, such as Chinese hamster ovary (CHO) cell.

The antibody or antigen binding fragment thereof, may be made using methods known in the art, for example, by a process comprising culturing a host cell containing nucleic acid encoding any of the previously described anti-PD-L1 antibodies or antigen-binding fragments in a form suitable for expression, under conditions suitable to produce such antibody or fragment, and recovering the antibody or fragment.

It is expressly contemplated that such PD-L1 axis binding antagonist antibodies (e.g., anti-PD-L1 antibodies, anti-PD-1 antibodies, and anti-PD-L2 antibodies), or other antibodies described herein for use in any of the aspects enumerated above may have any of the features, singly or in combination.

In some aspects, the PD-L1 axis binding antagonist is avelumab, durvalumab, cemiplimab, nivolumab, pembrolizumab, prolgolimab, camrelizumab, sintilimab, tislelizumab, toripalimab, dostarlimab, retifanlimab, spartalizumab, sasanlimab, penpulimab, CS1003, HLX10, SCT-110A, SHR-1316, CS1001, envafolimab, TQB2450, ZKAB001, LP-002, zimberelimab, balstilimab, genolimzumab, BI 754091, cetrelimab, YBL-006, BAT1306, HX008, CX-072, IMC-001, KL-A167, budigalimab, AMG 404, CX-188, JTX-4014, 609A, Sym021, LZM009, F520, SG001, APL-502, cosibelimab, lodapolimab, GS-4224, INCB086550, FAZ053, TG-1501, BGB-A333, BCD-135, AK-106, LDP, GR1405, HLX20, MSB2311, MAX-10181, RC98, BION-004, AM0001, CB201, ENUM 244C8, ENUM 388D4, AUNP-012, STI-1110, ADG104, AK-103, LBL-006, hAb21, AVA-004, PDL-GEX, INCB090244, K_{D}036, KY1003, LYN192, MT-6035, VXM10, YBL-007, ABSK041, GB7003, JS-003, or HS-636.

In some aspects, the immune checkpoint inhibitor is an antagonist directed against a co-inhibitory molecule (e.g., a CTLA-4 antagonist (e.g., an anti-CTLA-4 antibody), a TIM-3 antagonist (e.g., an anti-TIM-3 antibody), or a LAG-3 antagonist (e.g., an anti-LAG-3 antibody)), or any combination thereof.

ii. Anti-TIGIT Antagonist Antibodies

In some aspects, the immune checkpoint inhibitor is an antagonist directed against TIGIT (e.g., an anti-TIGIT antagonist antibody). Exemplary anti-TIGIT antagonist antibodies are described in US Patent Application Publication No. 2018/0186875 and in International Patent Application Publication No. WO 2017/053748, which are incorporated herein by reference in their entirety.

In some instances, the anti-TIGIT antagonist antibody is tiragolumab (CAS Registry Number: 1918185-84-8). Tiragolumab (Genentech) is also known as MTIG7192A.

In certain instances, the anti-TIGIT antagonist antibody includes at least one, two, three, four, five, or six HVRs selected from: (a) an HVR-H1 comprising the amino acid sequence of SNSAAWN (SEQ ID NO: 34); (b) an HVR-H2 comprising the amino acid sequence of KTYYRFKWYSDYAVSVKG (SEQ ID NO: 35); (c) an HVR-H3 comprising the amino acid sequence of ESTTYDLLAGPFDY (SEQ ID NO: 36); (d) an HVR-L1 comprising the amino acid sequence of KSSQTVLYSSNNKKYLA (SEQ ID NO: 37), (e) an HVR-L2 comprising the amino acid sequence of WASTRES (SEQ ID NO: 38); and/or (f) an HVR-L3 comprising the amino acid sequence of QQYYSTPFT (SEQ ID NO: 39), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 34-39.

In some instances, any of the above anti-TIGIT antagonist antibodies includes (a) an HVR-H1 comprising the amino acid sequence of SNSAAWN (SEQ ID NO: 34); (b) an HVR-H2 comprising the amino acid sequence of KTYYRFKWYSDYAVSVKG (SEQ ID NO: 35); (c) an HVR-H3 comprising the amino acid sequence of ESTTYDLLAGPFDY (SEQ ID NO: 36); (d) an HVR-L1 comprising the amino acid sequence of KSSQTVLYSSNNKKYLA (SEQ ID NO: 37); (e) an HVR-L2 comprising the amino acid sequence of WASTRES (SEQ ID NO: 38); and (f) an HVR-L3 comprising the amino acid sequence of QQYYSTPFT (SEQ ID NO: 39). In some instances, the anti-TIGIT antagonist antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of, EVQLQQSGPGLVKPSQTLSLTCAISGDSVSSNSAAWNWIRQSPSRGLEWLGKTYYRFKWYSDYAVSVKGRITIN PDTSKNQFSLQLNSVTPEDTAVFYCTRESTTYDLLAGPFDYWGQGTLVTVSS (SEQ ID NO: 50) or QVQLQQSGPGLVKPSQTLSLTCAISGDSVSSNSAAWNWIRQSPSRGLEWLGKTYYRFKWYSDYAVSVKGRITIN PDTSKNQFSLQLNSVTPEDTAVFYCTRESTTYDLLAGPFDYWGQGTLVTVSS (SEQ ID NO: 51) and/or a VL domain comprising an amino acid sequence having at least 90% sequence identity (e.g., at least 91%, 92%, 93%, 94%, 95%, 96%, 97%, 98%, or 99% sequence identity) to, or the sequence of, DIVMTQSPDSLAVSLGERATINCKSSQTVLYSSNNKKYLAWYQQKPGQPPNLLIYWASTRESGVPDRFSGSGSG TDFTLTISSLQAEDVAVYYCQQYYSTPFTFGPGTKVEIK (SEQ ID NO: 52). In some instances, the anti-TIGIT antagonist antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of, SEQ ID NO: 50 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 52. In some instances, the anti-TIGIT antagonist antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of, SEQ ID NO: 51 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 52.

In some instances, the anti-TIGIT antagonist antibody includes a heavy chain and a light chain sequence, wherein: (a) the heavy chain comprises the amino acid sequence:

(SEQ ID NO: 55)
EVQLQQSGPGLVKPSQTLSLTCAISGDSVSSNSAAWNWIR
QSPSRGLEWLGKTYYRFKWYSDYAVSVKGRITINPDTSKN
QFSLQLNSVTPEDTAVFYCTRESTTYDLLAGPFDYWGQGT
LVTVSSASTKGPSVFPLAPSSKSTSGGTAALGCLVKDYFP
EPVTVSWNSGALTSGVHTFPAVLQSSGLYSLSSVVTVPSS
SLGTQTYICNVNHKPSNTKVDKKVEPKSCDKTHTCPPCPA
PELLGGPSVFLFPPKPKDTLMISRTPEVTCVVVDVSHEDP
EVKFNWYVDGVEVHNAKTKPREEQYNSTYRVVSVLTVLHQ
DWLNGKEYKCKVSNKALPAPIEKTISKAKGQPREPQVYTL
PPSREEMTKNQVSLTCLVKGFYPSDIAVEWESNGQPENNY
KTTPPVLDSDGSFFLYSKLTVDKSRWQQGNVFSCSVMHEA
LHNHYTQKSLSLSPGK;

and (b) the light chain comprises the amino acid sequence:

(SEQ ID NO: 56)
DIVMTQSPDSLAVSLGERATINCKSSQTVLYSSNNKKYLA
WYQQKPGQPPNLLIYWASTRESGVPDRFSGSGSGTDFTLT
ISSLQAEDVAVYYCQQYYSTPFTFGPGTKVEIKRTVAAPS
VFIFPPSDEQLKSGTASVVCLLNNFYPREAKVQWKVDNAL
QSGNSQESVTEQDSKDSTYSLSSTLTLSKADYEKHKVYAC
EVTHQGLSSPVTKSFNRGEC.

In some instances, the anti-TIGIT antagonist antibody further comprises at least one, two, three, or four of the following light chain variable region framework regions (FRs): an FR-L1 comprising the amino acid sequence of DIVMTQSPDSLAVSLGERATINC (SEQ ID NO: 40); an FR-L2 comprising the amino acid sequence of WYQQKPGQPPNLLIY (SEQ ID NO: 41); an FR-L3 comprising the amino acid sequence of GVPDRFSGSGSGTDFTLTISSLQAEDVAVYYC (SEQ ID NO: 42); and/or an FR-L4 comprising the amino acid sequence of FGPGTKVEIK (SEQ ID NO: 43), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 40-43. In some instances, for example, the antibody further comprises an FR-L1 comprising the amino acid sequence of DIVMTQSPDSLAVSLGERATINC (SEQ ID NO: 40); an FR-L2 comprising the amino acid sequence of WYQQKPGQPPNLLIY (SEQ ID NO: 41); an FR-L3 comprising the amino acid sequence of GVPDRFSGSGSGTDFTLTISSLQAEDVAVYYC (SEQ ID NO: 42); and an FR-L4 comprising the amino acid sequence of FGPGTKVEIK (SEQ ID NO: 43).

In some instances, the anti-TIGIT antagonist antibody further comprises at least one, two, three, or four of the following heavy chain variable region FRs: an FR-H1 comprising the amino acid sequence of X₁VQLQQSGPGLVKPSQTLSLTCAISGDSVS (SEQ ID NO: 44), wherein X₁ is Q or E; an FR-H2 comprising the amino acid sequence of WIRQSPSRGLEWLG (SEQ ID NO: 45); an FR-H3 comprising the amino acid sequence of RITINPDTSKNQFSLQLNSVTPEDTAVFYCTR (SEQ ID NO: 46); and/or an FR-H4 comprising the amino acid sequence of WGQGTLVTVSS (SEQ ID NO: 47), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 44-47. The anti-TIGIT antagonist antibody may further include, for example, at least one, two, three, or four of the following heavy chain variable region FRs: an FR-H1 comprising the amino acid sequence of EVQLQQSGPGLVKPSQTLSLTCAISGDSVS (SEQ ID NO: 48); an FR-H2 comprising the amino acid sequence of WIRQSPSRGLEWLG (SEQ ID NO: 45); an FR-H3 comprising the amino acid sequence of RITINPDTSKNQFSLQLNSVTPEDTAVFYCTR (SEQ ID NO: 46); and/or an FR-H4 comprising the amino acid sequence of WGQGTLVTVSS (SEQ ID NO: 47), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 45-48. In some instances, the anti-TIGIT antagonist antibody includes an FR-H1 comprising the amino acid sequence of EVQLQQSGPGLVKPSQTLSLTCAISGDSVS (SEQ ID NO: 48); an FR-H2 comprising the amino acid sequence of WIRQSPSRGLEWLG (SEQ ID NO: 45); an FR-H3 comprising the amino acid sequence of RITINPDTSKNQFSLQLNSVTPEDTAVFYCTR (SEQ ID NO: 46); and an FR-H4 comprising the amino acid sequence of WGQGTLVTVSS (SEQ ID NO: 47).

In another instance, for example, the anti-TIGIT antagonist antibody may further include at least one, two, three, or four of the following heavy chain variable region FRs: an FR-H1 comprising the amino acid sequence of QVQLQQSGPGLVKPSQTLSLTCAISGDSVS (SEQ ID NO: 49); an FR-H2 comprising the amino acid sequence of WIRQSPSRGLEWLG (SEQ ID NO: 45); an FR-H3 comprising the amino acid sequence of RITINPDTSKNQFSLQLNSVTPEDTAVFYCTR (SEQ ID NO: 46); and/or an FR-H4 comprising the amino acid sequence of WGQGTLVTVSS (SEQ ID NO: 47), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 45-47 and 49. In some instances, the anti-TIGIT antagonist antibody includes an FR-H1 comprising the amino acid sequence of QVQLQQSGPGLVKPSQTLSLTCAISGDSVS (SEQ ID NO: 49); an FR-H2 comprising the amino acid sequence of WIRQSPSRGLEWLG (SEQ ID NO: 45); an FR-H3 comprising the amino acid sequence of RITINPDTSKNQFSLQLNSVTPEDTAVFYCTR (SEQ ID NO: 46); and an FR-H4 comprising the amino acid sequence of WGQGTLVTVSS (SEQ ID NO: 47).

In another aspect, an anti-TIGIT antagonist antibody is provided, wherein the antibody comprises a VH as in any of the instances provided above, and a VL as in any of the instances provided above, wherein one or both of the variable domain sequences include post-translational modifications.

In some instances, any one of the anti-TIGIT antagonist antibodies described above is capable of binding to rabbit TIGIT, in addition to human TIGIT. In some instances, any one of the anti-TIGIT antagonist antibodies described above is capable of binding to both human TIGIT and cynomolgus monkey (cyno) TIGIT. In some instances, any one of the anti-TIGIT antagonist antibodies described above is capable of binding to human TIGIT, cyno TIGIT, and rabbit TIGIT. In some instances, any one of the anti-TIGIT antagonist antibodies described above is capable of binding to human TIGIT, cyno TIGIT, and rabbit TIGIT, but not murine TIGIT.

In some instances, the anti-TIGIT antagonist antibody binds human TIGIT with a K_{D} of about 10 nM or lower and cyno TIGIT with a K_{D} of about 10 nM or lower (e.g., binds human TIGIT with a K_{D} of about 0.1 nM to about 1 nM and cyno TIGIT with a K_{D} of about 0.5 nM to about 1 nM, e.g., binds human TIGIT with a K_{D} of about 0.1 nM or lower and cyno TIGIT with a K_{D} of about 0.5 nM or lower).

In some instances, the anti-TIGIT antagonist antibody specifically binds TIGIT and inhibit or block TIGIT interaction with poliovirus receptor (PVR) (e.g., the antagonist antibody inhibits intracellular signaling mediated by TIGIT binding to PVR). In some instances, the antagonist antibody inhibits or blocks binding of human TIGIT to human PVR with an IC50 value of 10 nM or lower (e.g., 1 nM to about 10 nM). In some instances, the antagonist antibody inhibits or blocks binding of cyno TIGIT to cyno PVR with an IC50 value of 50 nM or lower (e.g., 1 nM to about 50 nM, e.g., 1 nM to about 5 nM).

In some instances, the methods or uses described herein may include using or administering an isolated anti-TIGIT antagonist antibody that competes for binding to TIGIT with any of the anti-TIGIT antagonist antibodies described above. For example, the method may include administering an isolated anti-TIGIT antagonist antibody that competes for binding to TIGIT with an anti-TIGIT antagonist antibody having the following six HVRs: (a) an HVR-H1 comprising the amino acid sequence of SNSAAWN (SEQ ID NO: 34); (b) an HVR-H2 comprising the amino acid sequence of KTYYRFKWYSDYAVSVKG (SEQ ID NO: 35); (c) an HVR-H3 comprising the amino acid sequence of ESTTYDLLAGPFDY (SEQ ID NO: 36); (d) an HVR-L1 comprising the amino acid sequence of KSSQTVLYSSNNKKYLA (SEQ ID NO: 37), (e) an HVR-L2 comprising the amino acid sequence of WASTRES (SEQ ID NO: 38); and (f) an HVR-L3 comprising the amino acid sequence of QQYYSTPFT (SEQ ID NO: 39). The methods described herein may also include administering an isolated anti-TIGIT antagonist antibody that binds to the same epitope as an anti-TIGIT antagonist antibody described above.

An anti-TIGIT antagonist antibody according to any of the above instances may be a monoclonal antibody, comprising a chimeric, humanized, or human antibody. In some instances, the anti-TIGIT antagonist antibody is tiragolumab. In one instance, an anti-TIGIT antagonist antibody is an antibody fragment, for example, a Fv, Fab, Fab′, scFv, diabody, or F(ab′)₂ fragment. In another instance, the antibody is a full-length antibody, e.g., an intact IgG antibody (e.g., an intact IgG1 antibody) or other antibody class or isotype as defined herein.

In some aspects, the method comprises administering at least two immune checkpoint inhibitors to the individual. In some aspects, for example, the method comprises administering a PD-L1 axis binding antagonist (e.g., atezolizumab) and an anti-TIGIT antagonist antibody (e.g., tiragolumab) to the individual.

### C. Methods of Delivery

The compositions utilized in the methods described herein (e.g., immune checkpoint inhibitors) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some aspects, an immune checkpoint inhibitor (e.g., a PD-L1 axis binding antagonist, e.g., atezolizumab) is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Immune checkpoint inhibitors (e.g., an immune checkpoint inhibitor described in Section IIIB herein, e.g., an antibody, binding polypeptide, and/or small molecule) described herein (and any additional therapeutic agent) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The immune checkpoint inhibitor need not be, but is optionally formulated with and/or administered concurrently with one or more agents currently used to prevent or treat the disorder in question, e.g., one or more of the agents provided in Section IIID herein. The effective amount of such other agents depends on the amount of the immune checkpoint inhibitor present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the treatment of a cancer, e.g., a cancer described in Section IIIA herein, the appropriate dosage of an immune checkpoint inhibitor, e.g., a PD-L1 axis binding antagonist, an anti-TIGIT antagonist antibody, an antagonist directed against a co-inhibitory molecule (e.g., a CTLA-4 antagonist (e.g., an anti-CTLA-4 antibody), a TIM-3 antagonist (e.g., an anti-TIM-3 antibody), or a LAG-3 antagonist (e.g., an anti-LAG-3 antibody)), or any combination thereof, described herein (when used alone or in combination with one or more other additional therapeutic agents) will depend on the type of disease to be treated, the severity and course of the disease, whether the PD-L1 axis binding antagonist is administered for preventive or therapeutic purposes, previous therapy, the patient's clinical history and response to the PD-L1 axis binding antagonist, and the discretion of the attending physician. The immune checkpoint inhibitor is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the immune checkpoint inhibitor). An initial higher loading dose, followed by one or more lower doses, may be administered. However, other dosage regimens may be useful. The progress of this therapy is easily monitored by conventional techniques and assays.

For example, as a general proposition, the therapeutically effective amount of an immune checkpoint inhibitor, for example, a PD-L1 axis binding antagonist antibody, an anti-TIGIT antagonist antibody, an anti-CTLA-4 antibody, an anti-TIM-3 antibody, or an anti-LAG-3 antibody, administered to human will be in the range of about 0.01 to about 50 mg/kg of patient body weight, whether by one or more administrations. In some aspects, the antibody used is about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20 mg/kg, about 0.01 mg/kg to about 15 mg/kg, about 0.01 mg/kg to about 10 mg/kg, about 0.01 mg/kg to about 5 mg/kg, or about 0.01 mg/kg to about 1 mg/kg administered daily, weekly, every two weeks, every three weeks, or monthly, for example. In some aspects, the antibody is administered at 15 mg/kg. However, other dosage regimens may be useful. In one aspect, an anti-PD-L1 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, or about 1800 mg on day 1 of 21-day cycles (every three weeks, q3w). In some aspects, the anti-PD-L1 antibody atezolizumab is administered at 1200 mg intravenously every three weeks (q3w). In some aspects, the anti-PD-L1 antibody atezolizumab is administered at a fixed dose of about 840 mg every two weeks (q2w). In some aspects, the anti-PD-L1 antibody atezolizumab is administered at a fixed dose of about 1680 mg every four weeks (q4w). In some aspects, the anti-TIGIT antagonist antibody is administered at a fixed dose of between about 30 mg to about 1200 mg (e.g., about 600 mg) every three weeks (q3w). In some aspects, the anti-TIGIT antagonist antibody is administered at a fixed dose of about 420 mg every two weeks (q2w). In some aspects, the anti-TIGIT antagonist antibody is administered at a fixed dose of about 840 mg every four weeks (q4w). The dose may be administered as a single dose or as multiple doses (e.g., 2 or 3 doses), such as infusions. The dose of the antibody administered in a combination treatment may be reduced as compared to a single treatment. The progress of this therapy is easily monitored by conventional techniques.

In some aspects, the individual has not been previously treated for the cancer. In other aspects, the individual has received at least one prior anti-cancer therapy, e.g., is 2L+. In some aspects, the individual has not been previously administered an immune checkpoint inhibitor.

In some aspects, the individual is a human. In some aspects, the individual is female. In some aspects, the individual is of European ancestry.

### D. Additional Therapeutic Agents

In some aspects, the immune checkpoint inhibitor used with one or more additional therapeutic agents, e.g., a combination therapy. In some aspects, the composition comprising the immune checkpoint inhibitor further comprises the additional therapeutic agent. In another aspect, the additional therapeutic agent is delivered in a separate composition. In some aspects, the one or more additional therapeutic agents comprise an immunomodulatory agent, an anti-neoplastic agent, a chemotherapeutic agent, a growth inhibitory agent, an anti-angiogenic agent, a radiation therapy, a cytotoxic agent, a cell-based therapy, or a combination thereof.

Combination therapies as described above encompass combined administration (wherein two or more therapeutic agents are included in the same or separate formulations) and separate administration (wherein administration of an immune checkpoint inhibitor (e.g., an immune checkpoint inhibitor described in Section IIIB herein, e.g., a PD-L1 axis binding antagonist) can occur prior to, simultaneously, and/or following, administration of the additional therapeutic agent or agents). In one aspect, administration of an immune checkpoint inhibitor and administration of an additional therapeutic agent occur within about one month, or within about one, two or three weeks, or within about one, two, three, four, five, or six days, of each other.

In some aspects, the additional therapeutic agent is carboplatin. In some aspects, the additional therapeutic agent is paclitaxel. In some aspects, the additional therapeutic agent is nab-paclitaxel. In some aspects, the additional therapeutic agent is bevacizumab. In some aspects, the additional therapeutic agent is sunitinib. In some aspects, the additional therapeutic agent is etoposide.

i. Immunomodulatory Agents

In some aspects, the additional therapeutic agent is an immunomodulatory agent. In some aspects, the immunomodulatory agent is a T cell-dependent bispecific antibody or an mRNA-based personalized cancer vaccine (PCV).

Ia. T-Cell-Dependent Bispecific Antibodies (TDBs)

In some aspects, the immunomodulatory agent is a T-cell-dependent bispecific antibody (TDB). In some aspects, the TDB may bind to two different epitopes of the T cell marker CD3 (e.g., CD3ε or CD3γ). In other aspects, the TDB may bind to two different targets, one of which is CD3, and the other of which is a second biological molecule, e.g., a cell surface antigen, e.g., a tumor antigen. Exemplary tumor antigens are described in U.S. Pub. No. 2010/0111856.

Ib. T-Cell Receptor Bispecific Targeting Domains

In some aspects, the immunomodulatory agent is a T-cell receptor bispecific. In some aspects, the T cell receptor bispecific comprises a first region comprising a T cell receptor (“TCR”). In some aspects, the TCR binds to a pMHC epitope. In some aspects, the T cell receptor bispecific further comprises a targeting domain that binds to a tumor antigen. In some aspects, the T-cell receptor bispecific is an Immune mobilizing monoclonal T-cell receptor Against Cancer (ImmTAC). (Oates and Jakobsen, Oncolmmunology, 2(2), 2013; WO2010133828).

Ic. NK-Engaging Bispecific Targeting Domains

In some aspects, the immunomodulatory agent is a NK-engaging bispecific. In some aspects, the NK-engaging bispecific comprises a first targeting domain binding to an epitope on a NK cell and a second targeting domain binding to a different target, e.g., a tumor antigen. In some aspects, the NK-engaging bispecific comprises a first targeting domain binding CD16a, a protein expressed on the surface of NK cells, and a second targeting domain binding the tumor marker CD30. In some aspects, the NK-engaging bispecific is an NK cell TandB®. In some aspects, the NK cell TandB® is AFM13 (Reusch et al., mAbs, 6(3):727-738; 2014; U.S. Pat. No. 7,129,330B1; U.S. Pat. No. 9,035,026B2; WO0111059A1). In some aspects, the NK-engaging bispecific comprises a first targeting domain binding CD16a and a second targeting domain binding epidermal growth factor receptor (EGFR) or EGFRvIII. In some aspects, the NK cell TandB® is AFM24. (Treder et al., Journal of Clinical Oncology, 34(15 suppl), 2016; Ellwanger et al., J Immunother Cancer, 3(Suppl 2): 219, 2015). In some aspects, the NK-engaging bispecific comprises a first targeting domain binding NKp46 and a second targeting domain binding a tumor antigen.

Id. Personalized Cancer Vaccines (PCVs)

In some aspects, the immunomodulatory agent is a personalized cancer vaccine (PCV). PCV is a method of treatment comprising inducing in a patient an immune response against one or more (e.g., 1, 2, 3, 4, 5. 10, 15, 20, 30, 40, 50, 100, 200, 300, 400, 500, or more than 500) cancer-specific somatic mutations present in cancer cells of the patient, as described, for example, in PCT Pub. Nos. WO2014/082729 and WO2012/159754, which are incorporated by reference herein in their entirety.

In some aspects, the immune response is against one or more (e.g., 1, 2, 3, 4, 5. 10, 15, 20, 30, 40, 50, 100, 200, 300, 400, 500, or more than 500) individual tumor mutations. It is estimated that 30-400 protein-changing somatic mutations, which may result in tumor-specific T cell epitopes, are present in a human cancer cell. These mutations comprise a patient's individual cancer mutation “signature” (WO2014/082729). Mutations may be collected from tumor cells, e.g., circulating tumor cells (CTCs), which may be isolated from, e.g., a biopsy or a blood sample. In some aspects, mutations are determined by comparing DNA sequences in healthy versus cancerous cells using next-generation sequencing. The transcriptome (RNA) may also be sequenced to determine which proteins are expressed by the cancer cells (WO2012/159754). Mutation-based antigens (or epitopes thereof) thus identified may be encoded by a nucleic acid, e.g., an RNA, e.g., an in vitro transcribed RNA. Said antigens or epitopes may be spaced by linkers or lined up head-to-tail (WO2014/082729).

In some aspects, treatment with the PCV may comprise inducing in a patient a first immune response against one or more tumor antigens and a second immune response against one or more mutation-based antigens as described above (WO2014/082729). The first and second immune responses may be administered simultaneously or sequentially. In some aspects, the first immune response is against a tumor antigen (e.g., a TAA) prevalent in multiple cancers or in the cancer to be treated. Induction of the first immune response may comprise, e.g., administration of one or more vaccine products selected from a set comprising pre-manufactured vaccine products, e.g., an RNA encoding a polypeptide comprising a tumor antigen or a fragment thereof (WO2014/082729).

Ie. Antibodies Targeting a Tumor Antigen, an Immune Checkpoint Protein, or a Lymphocyte Receptor

In some aspects, the immunomodulatory agent is an antibody or antibody fragment targeting a lymphocyte receptor (e.g., a marker of T cells, a T cell receptor protein, or a marker of NK cells), a dendritic cell receptor, a tumor antigen, an immune checkpoint component, or a T cell agonist or antagonist.

ii. Chemotherapeutic Agents

In some aspects, the additional therapeutic agent is a chemotherapeutic agent. A chemotherapeutic agent is a chemical compound useful in the treatment of cancer. Exemplary chemotherapeutic agents include, but are not limited to erlotinib (TARCEVA®, Genentech/OSI Pharm.), anti-hormonal agents that act to regulate or inhibit hormone action on tumors such as anti-estrogens and selective estrogen receptor modulators (SERMs), antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), or trastuzumab (HERCEPTIN®, Genentech), EGFR inhibitors (EGFR antagonists), tyrosine kinase inhibitors, and chemotherapeutic agents also include non-steroidal anti-inflammatory drugs (NSAIDs) with analgesic, antipyretic and anti-inflammatory effects.

iii. Growth Inhibitory Agents

In some aspects, the additional therapeutic agent is a growth inhibitory agent. Exemplary growth inhibitory agents include agents that block cell cycle progression at a place other than S phase, e.g., agents that induce G1 arrest (e.g., DNA alkylating agents such as tamoxifen, prednisone, dacarbazine, mechlorethamine, cisplatin, methotrexate, 5-fluorouracil, or ara-C) or M-phase arrest (e.g., vincristine, vinblastine, taxanes (e.g., paclitaxel and docetaxel), doxorubicin, epirubicin, daunorubicin, etoposide, or bleomycin).

iv. Radiation Therapies

In some aspects, the additional therapeutic agent is a radiation therapy. Radiation therapies include the use of directed gamma rays or beta rays to induce sufficient damage to a cell so as to limit its ability to function normally or to destroy the cell altogether. Typical treatments are given as a one-time administration and typical dosages range from 10 to 200 units (Grays) per day.

v. Cytotoxic Agents

In some aspects, the additional therapeutic agent is a cytotoxic agent, e.g., a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and antitumor or anticancer agents.

vi. Cell-Based Therapies

The additional therapeutic agent may by a cell-based therapy, e.g., an adoptive cell transfer (ACT) therapy. Cell-based therapies include CAR-T, NAR-T, and NEO-T.

The immunomodulatory agent may be a T cell transduced with a chimeric antigen receptor (CAR-T). In some aspects, the immunomodulatory agent is a natural killer cell transduced with a chimeric antigen receptor (NAR-T; CAR-NK). In some aspects, the chimeric antigen receptor (CAR) comprises an antigen-binding domain (e.g., an antibody or a fragment thereof; a T-cell receptor (TCR) or a fragment thereof) binding to a tumor antigen, a transmembrane domain, and one or more intracellular signaling domains, e.g., a primary signaling domain (e.g., CD3ζ) and/or a costimulatory signaling domain (e.g., CD28, 4-1 BB) (WO2017-114497; Hartmann et al., EMBO Molecular Medicine, 9(9), 2017). The intracellular signaling domain may act to activate cytotoxicity.

In some aspects, the CAR is introduced into a population of immune effector cells, e.g., T cells or NK cells. The population of immune effector cells may be prepared for CAR, e.g., by use of a flow-through module, as described in WO2017117112. The immune effector cells may be autologous, e.g., deriving from the patient, or allogenic, e.g., derived from a donor. In some aspects, CAR-T and NAR-T cells are introduced to a patient intravenously or intratumorally.

In some aspects, the immunomodulatory agent is a neoantigen T cell (NEO-T) therapy. In some aspects, the immunomodulatory agent is a T cell transduced with a native TCR specific to a tumor neoantigen (“neoantigen-specific TCR”). In some aspects, the tumor neoantigen is prevalent in multiple cancers or in the cancer to be treated. In other aspects, the tumor neoantigen is specific to the cancer of an individual patient. In some aspects, the neoantigen-specific TCR is discovered by sequencing of an individual patient's TCRs.

In some aspects, the neoantigen-specific TCR is introduced into a population of T cells. In some aspects, the T cells are autologous. In some aspects, the native TCR is replaced by the neoantigen-specific TCR using gene editing technology.

In some instances, the methods include administering to the individual an anti-cancer therapy other than, or in addition to, an immune checkpoint inhibitor, for example, a PD-L1 axis binding antagonist (e.g., an anti-neoplastic agent, a chemotherapeutic agent, a growth inhibitory agent, an anti-angiogenic agent, a radiation therapy, or a cytotoxic agent).

In some instances, the methods further involve administering to the patient an effective amount of an additional therapeutic agent. In some instances, the additional therapeutic agent is selected from the group consisting of an anti-neoplastic agent, a chemotherapeutic agent, a growth inhibitory agent, an anti-angiogenic agent, a radiation therapy, a cytotoxic agent, and combinations thereof. In some instances, an immune checkpoint inhibitor, for example, a PD-L1 axis binding antagonist, may be administered in conjunction with a chemotherapy or chemotherapeutic agent. In some instances, an immune checkpoint inhibitor, for example, a PD-L1 axis binding antagonist, may be administered in conjunction with a radiation therapy agent. In some instances, an immune checkpoint inhibitor, for example, a PD-L1 axis binding antagonist, may be administered in conjunction with a targeted therapy or targeted therapeutic agent. In some instances, an immune checkpoint inhibitor, for example, a PD-L1 axis binding antagonist, may be administered in conjunction with an immunotherapy or immunotherapeutic agent, for example a monoclonal antibody. In some instances, the additional therapeutic agent is an agonist directed against a co-stimulatory molecule. In some instances, the additional therapeutic agent is an antagonist directed against a co-inhibitory molecule. In some instances, the PD-L1 axis binding antagonist is administered as a monotherapy.

Such combination therapies noted above encompass combined administration (where two or more therapeutic agents are included in the same or separate formulations), and separate administration, in which case, administration of an immune checkpoint inhibitor, for example, a PD-L1 axis binding antagonist, can occur prior to, simultaneously, and/or following, administration of the additional therapeutic agent or agents. In one instance, administration of PD-L1 axis binding antagonist and administration of an additional therapeutic agent occur within about one month, or within about one, two or three weeks, or within about one, two, three, four, five, or six days, of each other.

Without wishing to be bound to theory, it is thought that enhancing T-cell stimulation, by promoting a co-stimulatory molecule or by inhibiting a co-inhibitory molecule, may promote tumor cell death thereby treating or delaying progression of cancer. In some instances, an immune checkpoint inhibitor, e.g., an immune checkpoint inhibitor described in Section IIIB herein, may be administered in conjunction with an agonist directed against a co-stimulatory molecule. In some instances, a co-stimulatory molecule may include CD40, CD226, CD28, OX40, GITR, CD137, CD27, HVEM, or CD127. In some instances, the agonist directed against a co-stimulatory molecule is an agonist antibody that binds to CD40, CD226, CD28, OX40, GITR, CD137, CD27, HVEM, or CD127. In some instances, an immune checkpoint inhibitor, for example, a PD-L1 axis binding antagonist, may be administered in conjunction with an antagonist directed against a co-inhibitory molecule. In some instances, a co-inhibitory molecule may include CTLA-4 (also known as CD152), TIM-3, BTLA, VISTA, LAG-3, B7-H3, B7-H4, IDO, TIGIT, MICA/B, or arginase. In some instances, the antagonist directed against a co-inhibitory molecule is an antagonist antibody that binds to CTLA-4, TIM-3, BTLA, VISTA, LAG-3, B7-H3, B7-H4, IDO, TIGIT, MICA/B, or arginase.

In some instances, a PD-L1 axis binding antagonist may be administered in conjunction with an antagonist directed against CTLA-4 (also known as CD152), e.g., a blocking antibody. In some instances, a PD-L1 axis binding antagonist may be administered in conjunction with ipilimumab (also known as MDX-010, MDX-101, or YERVOY®). In some instances, a PD-L1 axis binding antagonist may be administered in conjunction with tremelimumab (also known as ticilimumab or CP-675,206). In some instances, a PD-L1 axis binding antagonist may be administered in conjunction with an antagonist directed against B7-H3 (also known as CD276), e.g., a blocking antibody. In some instances, a PD-L1 axis binding antagonist may be administered in conjunction with MGA271. In some instances, a PD-L1 axis binding antagonist may be administered in conjunction with an antagonist directed against a TGF-beta, e.g., metelimumab (also known as CAT-192), fresolimumab (also known as GC1008), or LY2157299.

In some instances, an immune checkpoint inhibitor, for example, a PD-L1 axis binding antagonist, may be administered in conjunction with a treatment comprising adoptive transfer of a T-cell (e.g., a cytotoxic T-cell or CTL) expressing a chimeric antigen receptor (CAR). In some instances, an immune checkpoint inhibitor, for example, a PD-L1 axis binding antagonist, may be administered in conjunction with a treatment comprising adoptive transfer of a T-cell comprising a dominant-negative TGF beta receptor, e.g., a dominant-negative TGF beta type II receptor. In some instances, an immune checkpoint inhibitor, for example, a PD-L1 axis binding antagonist, may be administered in conjunction with a treatment comprising a HERCREEM protocol (see, e.g., ClinicalTrials.gov Identifier NCT00889954).

In some instances, an immune checkpoint inhibitor, for example, a PD-L1 axis binding antagonist, may be administered in conjunction with an agonist directed against CD137 (also known as TNFRSF9, 4-1 BB, or ILA), e.g., an activating antibody. In some instances, an immune checkpoint inhibitor, for example, a PD-L1 axis binding antagonist, may be administered in conjunction with urelumab (also known as BMS-663513). In some instances, an immune checkpoint inhibitor, for example, a PD-L1 axis binding antagonist, may be administered in conjunction with an agonist directed against CD40, e.g., an activating antibody. In some instances, an immune checkpoint inhibitor, for example, a PD-L1 axis binding antagonist, may be administered in conjunction with CP-870893. In some instances, an immune checkpoint inhibitor, for example, a PD-L1 axis binding antagonist, may be administered in conjunction with an agonist directed against OX40 (also known as CD134), e.g., an activating antibody. In some instances, an immune checkpoint inhibitor, for example, a PD-L1 axis binding antagonist, may be administered in conjunction with an anti-OX40 antibody (e.g., AgonOX). In some instances, an immune checkpoint inhibitor, for example, a PD-L1 axis binding antagonist, may be administered in conjunction with an agonist directed against CD27, e.g., an activating antibody. In some instances, an immune checkpoint inhibitor, for example, a PD-L1 axis binding antagonist, may be administered in conjunction with CDX-1127. In some instances, an immune checkpoint inhibitor, for example, a PD-L1 axis binding antagonist, may be administered in conjunction with an antagonist directed against indoleamine-2,3-dioxygenase (IDO). In some instances, with the IDO antagonist is 1-methyl-D-tryptophan (also known as 1-D-MT).

In some instances, an immune checkpoint inhibitor, for example, a PD-L1 axis binding antagonist, may be administered in conjunction with an antibody-drug conjugate. In some instances, the antibody-drug conjugate comprises mertansine or monomethyl auristatin E (MMAE). In some instances, an immune checkpoint inhibitor, for example, a PD-L1 axis binding antagonist, may be administered in conjunction with an anti-NaPi2b antibody-MMAE conjugate (also known as DNIB0600A or RG7599). In some instances, an immune checkpoint inhibitor, for example, a PD-L1 axis binding antagonist, may be administered in conjunction with trastuzumab emtansine (also known as T-DM1, ado-trastuzumab emtansine, or KADCYLA®, Genentech). In some instances, an immune checkpoint inhibitor, for example, a PD-L1 axis binding antagonist, may be administered in conjunction with DMUC5754A. In some instances, an immune checkpoint inhibitor, for example, a PD-L1 axis binding antagonist, may be administered in conjunction with an antibody-drug conjugate targeting the endothelin B receptor (EDNBR), e.g., an antibody directed against EDNBR conjugated with MMAE.

In some instances, an immune checkpoint inhibitor, for example, a PD-L1 axis binding antagonist, may be administered in conjunction with an anti-angiogenesis agent. In some instances, an immune checkpoint inhibitor, for example, a PD-L1 axis binding antagonist, may be administered in conjunction with an antibody directed against a VEGF, e.g., VEGF-A. In some instances, an immune checkpoint inhibitor, for example, a PD-L1 axis binding antagonist, may be administered in conjunction with bevacizumab (also known as AVASTIN®, Genentech). In some instances, an immune checkpoint inhibitor, for example, a PD-L1 axis binding antagonist, may be administered in conjunction with an antibody directed against angiopoietin 2 (also known as Ang2). In some instances, an immune checkpoint inhibitor, for example, a PD-L1 axis binding antagonist, may be administered in conjunction with MEDI3617.

In some instances, an immune checkpoint inhibitor, for example, a PD-L1 axis binding antagonist, may be administered in conjunction with an antineoplastic agent. In some instances, an immune checkpoint inhibitor, for example, a PD-L1 axis binding antagonist, may be administered in conjunction with an agent targeting CSF-1R (also known as M-CSFR or CD115). In some instances, an immune checkpoint inhibitor, for example, a PD-L1 axis binding antagonist, may be administered in conjunction with anti-CSF-1R (also known as IMC-CS4). In some instances, an immune checkpoint inhibitor, for example, a PD-L1 axis binding antagonist, may be administered in conjunction with an interferon, for example interferon alpha or interferon gamma. In some instances, an immune checkpoint inhibitor, for example, a PD-L1 axis binding antagonist, may be administered in conjunction with Roferon-A (also known as recombinant Interferon alpha-2a). In some instances, an immune checkpoint inhibitor, for example, a PD-L1 axis binding antagonist, may be administered in conjunction with GM-CSF (also known as recombinant human granulocyte macrophage colony stimulating factor, rhu GM-CSF, sargramostim, or LEUKINE®). In some instances, an immune checkpoint inhibitor, for example, a PD-L1 axis binding antagonist, may be administered in conjunction with IL-2 (also known as aldesleukin or PROLEUKIN®). In some instances, an immune checkpoint inhibitor, for example, a PD-L1 axis binding antagonist, may be administered in conjunction with IL-12. In some instances, an immune checkpoint inhibitor, for example, a PD-L1 axis binding antagonist, may be administered in conjunction with an antibody targeting CD20. In some instances, the antibody targeting CD20 is obinutuzumab (also known as GA101 or GAZYVA®) or rituximab. In some instances, an immune checkpoint inhibitor, for example, a PD-L1 axis binding antagonist, may be administered in conjunction with an antibody targeting GITR. In some instances, the antibody targeting GITR is TRX518.

In some instances, an immune checkpoint inhibitor, for example, a PD-L1 axis binding antagonist, may be administered in conjunction with a cancer vaccine. In some instances, the cancer vaccine is a peptide cancer vaccine, which in some instances is a personalized peptide vaccine. In some instances the peptide cancer vaccine is a multivalent long peptide, a multi-peptide, a peptide cocktail, a hybrid peptide, or a peptide-pulsed dendritic cell vaccine (see, e.g., Yamada et al., Cancer Sci. 104:14-21, 2013). In some instances, an immune checkpoint inhibitor, for example, a PD-L1 axis binding antagonist, may be administered in conjunction with an adjuvant. In some instances, an immune checkpoint inhibitor, for example, a PD-L1 axis binding antagonist, may be administered in conjunction with a treatment comprising a TLR agonist, e.g., Poly-ICLC (also known as HILTONOL®), LPS, MPL, or CpG ODN. In some instances, an immune checkpoint inhibitor, for example, a PD-L1 axis binding antagonist, may be administered in conjunction with tumor necrosis factor (TNF) alpha. In some instances, an immune checkpoint inhibitor, for example, a PD-L1 axis binding antagonist, may be administered in conjunction with IL-1. In some instances, an immune checkpoint inhibitor, for example, a PD-L1 axis binding antagonist, may be administered in conjunction with HMGB1. In some instances, an immune checkpoint inhibitor, for example, a PD-L1 axis binding antagonist, may be administered in conjunction with an IL-10 antagonist. In some instances, an immune checkpoint inhibitor, for example, a PD-L1 axis binding antagonist, may be administered in conjunction with an IL-4 antagonist. In some instances, an immune checkpoint inhibitor, for example, a PD-L1 axis binding antagonist, may be administered in conjunction with an IL-13 antagonist. In some instances, an immune checkpoint inhibitor, for example, a PD-L1 axis binding antagonist, may be administered in conjunction with an HVEM antagonist. In some instances, an immune checkpoint inhibitor, for example, a PD-L1 axis binding antagonist, may be administered in conjunction with an ICOS agonist, e.g., by administration of ICOS-L, or an agonistic antibody directed against ICOS. In some instances, an immune checkpoint inhibitor, for example, a PD-L1 axis binding antagonist, may be administered in conjunction with a treatment targeting CX3CL1. In some instances, an immune checkpoint inhibitor, for example, a PD-L1 axis binding antagonist, may be administered in conjunction with a treatment targeting CXCL9. In some instances, an immune checkpoint inhibitor, for example, a PD-L1 axis binding antagonist, may be administered in conjunction with a treatment targeting CXCL10. In some instances, an immune checkpoint inhibitor, for example, a PD-L1 axis binding antagonist, may be administered in conjunction with a treatment targeting CCL5. In some instances, an immune checkpoint inhibitor, for example, a PD-L1 axis binding antagonist, may be administered in conjunction with an LFA-1 or ICAM1 agonist. In some instances, an immune checkpoint inhibitor, for example, a PD-L1 axis binding antagonist, may be administered in conjunction with a Selectin agonist.

In some instances, an immune checkpoint inhibitor, for example, a PD-L1 axis binding antagonist, may be administered in conjunction with a targeted therapy. In some instances, an immune checkpoint inhibitor, for example, a PD-L1 axis binding antagonist, may be administered in conjunction with an inhibitor of B-Raf. In some instances, an immune checkpoint inhibitor, for example, a PD-L1 axis binding antagonist, may be administered in conjunction with vemurafenib (also known as ZELBORAF®). In some instances, an immune checkpoint inhibitor, for example, a PD-L1 axis binding antagonist, may be administered in conjunction with dabrafenib (also known as TAFINLAR®). In some instances, an immune checkpoint inhibitor, for example, a PD-L1 axis binding antagonist, may be administered in conjunction with erlotinib (also known as TARCEVA®). In some instances, an immune checkpoint inhibitor, for example, a PD-L1 axis binding antagonist, may be administered in conjunction with an inhibitor of a MEK, such as MEK1 (also known as MAP2K1) or MEK2 (also known as MAP2K2). In some instances, an immune checkpoint inhibitor, for example, a PD-L1 axis binding antagonist, may be administered in conjunction with cobimetinib (also known as GDC-0973 or XL-518). In some instances, an immune checkpoint inhibitor, for example, a PD-L1 axis binding antagonist, may be administered in conjunction with trametinib (also known as MEKINIST®). In some instances, an immune checkpoint inhibitor, for example, a PD-L1 axis binding antagonist, may be administered in conjunction with an inhibitor of K-Ras. In some instances, an immune checkpoint inhibitor, for example, a PD-L1 axis binding antagonist, may be administered in conjunction with an inhibitor of c-Met. In some instances, an immune checkpoint inhibitor, for example, a PD-L1 axis binding antagonist, may be administered in conjunction with onartuzumab (also known as MetMAb). In some instances, an immune checkpoint inhibitor, for example, a PD-L1 axis binding antagonist, may be administered in conjunction with an inhibitor of Alk. In some instances, an immune checkpoint inhibitor, for example, a PD-L1 axis binding antagonist, may be administered in conjunction with AF802 (also known as CH5424802 or alectinib). In some instances, an immune checkpoint inhibitor, for example, a PD-L1 axis binding antagonist, may be administered in conjunction with an inhibitor of a phosphatidylinositol 3-kinase (PI3K). In some instances, an immune checkpoint inhibitor, for example, a PD-L1 axis binding antagonist, may be administered in conjunction with BKM120. In some instances, an immune checkpoint inhibitor, for example, a PD-L1 axis binding antagonist, may be administered in conjunction with idelalisib (also known as GS-1101 or CAL-101). In some instances, an immune checkpoint inhibitor, for example, a PD-L1 axis binding antagonist, may be administered in conjunction with perifosine (also known as KRX-0401). In some instances, an immune checkpoint inhibitor, for example, a PD-L1 axis binding antagonist, may be administered in conjunction with an inhibitor of an Akt. In some instances, an immune checkpoint inhibitor, for example, a PD-L1 axis binding antagonist, may be administered in conjunction with MK2206. In some instances, an immune checkpoint inhibitor, for example, a PD-L1 axis binding antagonist, may be administered in conjunction with GSK690693. In some instances, an immune checkpoint inhibitor, for example, a PD-L1 axis binding antagonist, may be administered in conjunction with GDC-0941. In some instances, an immune checkpoint inhibitor, for example, a PD-L1 axis binding antagonist, may be administered in conjunction with an inhibitor of mTOR. In some instances, an immune checkpoint inhibitor, for example, a PD-L1 axis binding antagonist, may be administered in conjunction with sirolimus (also known as rapamycin). In some instances, an immune checkpoint inhibitor, for example, a PD-L1 axis binding antagonist, may be administered in conjunction with temsirolimus (also known as CCI-779 or TORISEL®). In some instances, an immune checkpoint inhibitor, for example, a PD-L1 axis binding antagonist, may be administered in conjunction with everolimus (also known as RAD001). In some instances, an immune checkpoint inhibitor, for example, a PD-L1 axis binding antagonist, may be administered in conjunction with ridaforolimus (also known as AP-23573, MK-8669, or deforolimus). In some instances, an immune checkpoint inhibitor, for example, a PD-L1 axis binding antagonist, may be administered in conjunction with OSI-027. In some instances, an immune checkpoint inhibitor, for example, a PD-L1 axis binding antagonist, may be administered in conjunction with AZD8055. In some instances, an immune checkpoint inhibitor, for example, a PD-L1 axis binding antagonist, may be administered in conjunction with INK128. In some instances, an immune checkpoint inhibitor, for example, a PD-L1 axis binding antagonist, may be administered in conjunction with a dual PI3K/mTOR inhibitor. In some instances, an immune checkpoint inhibitor, for example, a PD-L1 axis binding antagonist, may be administered in conjunction with XL765. In some instances, an immune checkpoint inhibitor, for example, a PD-L1 axis binding antagonist, may be administered in conjunction with GDC-0980. In some instances, an immune checkpoint inhibitor, for example, a PD-L1 axis binding antagonist, may be administered in conjunction with BEZ235 (also known as NVP-BEZ235). In some instances, an immune checkpoint inhibitor, for example, a PD-L1 axis binding antagonist, may be administered in conjunction with BGT226. In some instances, an immune checkpoint inhibitor, for example, a PD-L1 axis binding antagonist, may be administered in conjunction with GSK2126458. In some instances, an immune checkpoint inhibitor, for example, a PD-L1 axis binding antagonist, may be administered in conjunction with PF-04691502. In some instances, an immune checkpoint inhibitor, for example, a PD-L1 axis binding antagonist, may be administered in conjunction with PF-05212384 (also known as PKI-587).

### IV. Articles of Manufacture and Kits

In another aspect of the invention, an article of manufacture or kit containing materials useful for the prognostic assessment and/or treatment of individuals is provided.

In some aspects, the disclosure features a kit for identifying an individual having a cancer who has an increased likelihood of experiencing treatment-induced thyroid dysfunction during treatment comprising an immune checkpoint inhibitor (e.g., a PD-L1 axis binding antagonist (e.g., a PD-L1 binding antagonist, e.g., atezolizumab)), the kit comprising (a) polypeptides or polynucleotides for determining the presence of a set of risk alleles selected from independent genetic signals in a GWAS for hypothyroidism; and/or (b) polypeptides or polynucleotides capable of determining the presence of a set of risk alleles selected from independent genetic signals in a GWAS for vitiligo; and (c) instructions for use of the polypeptides or polynucleotides to determine a polygenic risk score (PRS) for one or both of hypothyroidism and vitiligo from a sample from the individual, wherein (i) a PRS for hypothyroidism that is above a hypothyroidism reference PRS or (ii) a PRS for vitiligo that is above a vitiligo reference PRS identifies the individual as one who may have an increased likelihood of experiencing treatment-induced thyroid dysfunction during treatment comprising a PD-L1 axis binding antagonist.

In some aspects, the risk alleles are selected from Table 7 or Table 8.

In some aspects, the PD-L1 binding antagonist is atezolizumab (MPDL3280A).

In some aspects, the disclosure features a kit for identifying an individual having a TNBC who may benefit from a treatment comprising an immune checkpoint inhibitor (e.g., a PD-L1 axis binding antagonist (e.g., a PD-L1 binding antagonist, e.g., atezolizumab)), the kit comprising (a) polypeptides or polynucleotides for determining the presence of a set of risk alleles selected from independent genetic signals in a GWAS for hypothyroidism; and (b) instructions for use of the polypeptides or polynucleotides to determine a polygenic risk score (PRS) for hypothyroidism from a sample from the individual, wherein a PRS for hypothyroidism that is above a hypothyroidism reference PRS identifies the individual as one who may benefit from a treatment comprising a PD-L1 axis binding antagonist.

In some aspects, the risk alleles are selected from Table 7 or Table 8.

In some aspects, the PD-L1 binding antagonist is atezolizumab (MPDL3280A).

In some instances, such articles of manufacture or kits can be used to identify an individual having a breast cancer (e.g., a TNBC) who may benefit from treatment with an immune checkpoint inhibitor, e.g., an immune checkpoint inhibitor described in Section IIIB herein, e.g., a PD-L1 axis binding antagonist and/or an anti-TIGIT antagonist antibody. Such articles of manufacture or kits may include (a) reagents for determining the polygenic risk score (PRS) of an individual for hypothyroidism, as described in Section IIA, and (b) instructions for using the reagents to identify an individual having a breast cancer (e.g., a TNBC) who may benefit from a treatment comprising an immune checkpoint inhibitor, e.g., an immune checkpoint inhibitor described in Section IIIB herein, e.g., a PD-L1 axis binding antagonist and/or an anti-TIGIT antagonist antibody.

In some aspects, such articles of manufacture or kits include an immune checkpoint inhibitor, e.g., an immune checkpoint inhibitor described in Section IIIB herein, for treating an individual with a cancer (e.g., breast cancer, e.g., TNBC). In some aspects, the article of manufacture or kit includes (a) an immune checkpoint inhibitor, e.g., an immune checkpoint inhibitor described in Section IIIB herein and (b) a package insert including instructions for administration of the immune checkpoint inhibitor to an individual having a cancer (e.g., breast cancer, e.g., TNBC), wherein, prior to treatment, the polygenic risk score (PRS) of an individual for hypothyroidism in a sample from the individual has been determined and is the same as or above a hypothyroidism reference PRS.

Any of the articles of manufacture or kits described may include a carrier means being compartmentalized to receive in close confinement one or more container means such as vials, tubes, and the like, each of the container means comprising one of the separate elements to be used in the method. Where the article of manufacture or kit utilizes nucleic acid hybridization to detect the target nucleic acid, the kit may also have containers containing nucleotide(s) for amplification of the target nucleic acid sequence and/or a container comprising a reporter-means, such as an enzymatic, fluorescent, or radioisotope label.

In some aspects, the article of manufacture or kit includes the container described above and one or more other containers including materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above. For example, the article of manufacture or kit may further include a container including a pharmaceutically-acceptable buffer, such as bacteriostatic water for injection (BWFI), phosphate-buffered saline, Ringer's solution, and dextrose solution.

The articles of manufacture or kits described herein may have a number of aspects. In one aspect, the article of manufacture or kit includes a container, a label on said container, and a composition contained within said container, wherein the composition includes one or more polynucleotides that hybridize to a complement of a locus described herein under stringent conditions, and the label on said container indicates that the composition can be used to evaluate the presence of a gene listed herein in a sample, or of a single-nucleotide polymorphism (SNP) described herein in a sample, and wherein the kit includes instructions for using the polynucleotide(s) for evaluating the presence of the gene RNA or DNA or the presence of the SNP in a particular sample type.

For oligonucleotide-based articles of manufacture or kits, the article of manufacture or kit can include, for example: (1) an oligonucleotide, e.g., a detectably labeled oligonucleotide, which hybridizes to a nucleic acid sequence encoding a protein or (2) a pair of primers useful for amplifying a nucleic acid molecule. The article of manufacture or kit can also include, e.g., a buffering agent, a preservative, or a protein stabilizing agent. The article of manufacture or kit can further include components necessary for detecting the detectable label (e.g., an enzyme or a substrate). The article of manufacture or kit can further include components necessary for analyzing the sequence of a sample (e.g., a restriction enzyme or a buffer). The article of manufacture or kit can also contain a control sample or a series of control samples that can be assayed and compared to the test sample. Each component of the article of manufacture or kit can be enclosed within an individual container and all of the various containers can be within a single package, along with instructions for interpreting the results of the assays performed using the kit.

### V. Examples

The following are examples of methods and compositions of the invention. It is understood that various other aspects may be practiced, given the general description provided above, and the examples are not intended to limit the scope of the claims.

### Example 1. Treatment with Anti-PD-L1 Therapy is Associated with Increased Risk of Thyroid Dysfunction

i. Introduction

PD-1 checkpoint inhibitors have made significant advances in the treatment of cancer. Considerable progress has been made in identifying the immune mechanisms that are responsible for the therapeutic benefit observed. These include the enhancement of T-cell priming and activation at the level of dendritic cells and the re-invigoration of “exhausted” intra-tumoral T-cells (Pauken et al., Trends Immunol., 36: 265-76, 2015; Oh et al., Nat. Cancer, 1: 681-691, 2020). However, PD-1 checkpoint inhibitors act systemically and patients can develop immune toxicities and rheumatic complications, termed immune-related adverse events (irAEs). Activation of the immune system due to diminished activity of the PD-1 checkpoint is hypothesized to contribute to autoimmunity that increases risk for irAEs, but the underlying risk factors and mechanisms are poorly understood (Postow et al., N. Engl. J. Med., 378: 158-168, 2018; June et al., Nat. Med., 23: 540-547, 2017; Calabrese et al., Nat. Rev. Rheumatol., 14: 569-579, 2018). In cancer patients treated with PD-1 checkpoint inhibitors, there exists considerable inter-individual variation in tumor response and immune toxicity. Understanding an individual and their tumor's immunological status, or cancer-immune set point, can explain this variation and identify therapeutic approaches to improve not only efficacy, but also safety (Chen and Mellman, Nature, 541: 321-30, 2017). This approach requires consideration of factors both intrinsic and extrinsic to a tumor, including genetic variation that affects the immune system.

Classes of dermatological and endocrine system irAEs that occur during treatment with PD-1 checkpoint inhibitors as monotherapy have been associated with longer survival in melanoma, suggesting these irAEs may also reflect immune responses associated with anti-tumor immunity (Freeman-Keller et al., Clin. Cancer Res., 22: 886-894, 2016; Teulings et al., J. Clin. Oncol., 33: 773-781, 2015). A recent study has confirmed these findings by accounting for sources of survival bias that arise when using adverse event data and by comparison to matched melanoma patients receiving placebo (Eggermont et al., JAMA Oncol., 6(4): 519-527, 2020). Yet, the clinical development path of PD-1 checkpoint inhibitors has focused on combination therapies. Recent clinical trials have demonstrated that adding immunotherapy to chemotherapy can improve clinical outcomes across cancers, supporting the notion that immune activation provides benefit beyond inducing cell death and inhibiting angiogenesis in tumors.

Endocrinopathies are common among patients that receive immune checkpoint inhibitors and rarely occur in patients treated with chemotherapies. While most dermatological irAEs are reversible, endocrinopathies can be permanent, representing a distinct risk for a cancer patient (Brahmer et al., J. Clin. Oncol., 36: 1714-1768, 2018). Studies have identified physiological risk factors for endocrine irAEs, yet the general applicability of these findings to chemotherapy combinations and underlying genes and mechanisms remain unclear (Kotwal et al., Thyroid, 30: 177-184, 2020; Osorio et al., Ann. Oncol., 28: 583-589, 2017; Byun et al., Nat. Rev. Endocrinol., 13: 195-207, 2017). Derived from disease-associated genomic loci, polygenic risk scores (PRS) have the potential to address these limitations. We recently introduced the use of PRS to determine the extent to which genetic variation shared with autoimmune disease impacts dermatological irAEs and survival in a single atezolizumab monotherapy bladder cancer trial (Khan et al., Proc. Natl. Acad. Sci., 117: 1228812294, 2020). The present study shows that thyroid irAEs are associated with lower risk of death across cancers and chemotherapy combinations with atezolizumab. To gain further insight into these irAEs, a PRS derived from a hypothyroidism GWAS is used to demonstrate that genetic variation affecting lifetime risk for autoimmune thyroid disease also impacts risk of thyroid irAEs during the shorter duration of atezolizumab treatment. Sparse regression analysis indicates that a subset of variants in this PRS drive this association and are in loci near genes involved in autoimmunity and regulation of immune responses, including responses driven by T-follicular-helper (Tfh) cells. The PRS was directly associated with lower risk of death in triple negative breast cancer (TNBC) patients treated with atezolizumab and nab-paclitaxel, suggesting that immune mechanisms that lead to thyroid autoimmunity may improve survival in these patients. This study emphasizes the importance of genetic variation affecting the immune system during cancer immunotherapy, with implications for the use of PRS in clinical decision-making and for the prioritization of immunotherapy targets.

ii. Thyroid irAE are Common and are Associated with Longer Survival

Activation of systemic immune responses using PD-1 checkpoint inhibitors is an essential approach to cancer therapy. Yet, the extent of benefit relative to risk of immune related adverse events (irAEs) varies widely between patients. To characterize their prevalence and clinical course, endocrine system irAEs were aggregated across the safety evaluable populations of seven phase 3 trials testing atezolizumab combinations with chemotherapies and bevacizumab and spanning six cancer indications: metastatic urothelial carcinoma (IMvigor211 (Powles, et al., Lancet, 391: 748-757, 2018)), squamous and non-squamous non-small cell lung cancer (IMpower150 (Socinski et al., N. Engl. J. Med., 378: 2288-2301, 2018)), IMpoweri30 (West et al., Lancet Oncol., 20: 924-937, 2019), IMpower131 (Jotte et al., J. Thorac. Oncol., 15(8): 1351-1360, 2020)), small cell lung cancer (IMpoweri33 (Horn et al, N. Engl. J. Med., 379: 2220-2229, 2018)), metastatic renal cell carcinoma (IMmotion151 (Rini et al., Lancet, 393: 2404-2415, 2019)), and triple-negative breast cancer (TNBC) (IMpassion130 (Schmid et al., N. Engl. J. Med., 379: 2108-2121, 2018; Schmid et al., Lancet Oncol., 21: 44-59, 2020)). (Tables 3 and 4). Protocols for each trial are provided in the original study publications (Table 3).

TABLE 3
Atezolizumab trials

| Trial | Abbreviation | Indication | Citation | NCT Number |
|---|---|---|---|---|
| IMvigor211 | imv211 | 2L metastatic | Powles, et al., Lancet, 391: | NCT02302807 |
| | | urothelial carcinoma | 748-757, 2018. | |
| IMpower150 | imp150 | 1L non-squamous | Socinski et al., N. Engl. J. | NCT02366143 |
| | | NSCLC | Med., 378: 2288-2301, 2018. | |
| IMpower131 | imp131 | 1L squamous | Jotte et al., J. Thorac. Oncol., | NCT02367794 |
| | | NSCLC | 15(8): 1351-1360, 2020. | |
| IMpower130 | imp130 | 1L non-squamous | West et al., Lancet Oncol., | NCT02367781 |
| | | NSCLC | 20: 924-937, 2019. | |
| IMpower133 | imp133 | SCLC | Horn et al, N. Engl. J. Med., | NCT02763579 |
| | | | 379: 2220-2229, 2018. | |
| IMmotion151 | imm151 | RCC | Rini et al., Lancet, 393: | NCT02420821 |
| | | | 2404-2415, 2019. | |
| IMpassion130 | impas130 | TNBC | Schmid et al., N. Engl. J. | NCT02425891 |
| | | | Med., 379: 2108-2121, 2018; | |
| | | | Schmid et al., Lancet Oncol., | |
| | | | 21: 44-59, 2020. | |

NSCLC: non-small cell lung cancer; SCLC: small cell lung cancer; RCC: renal cell carcinoma; TNBC = triple negative breast cancer; 1L = first line, 2L = second line

TABLE 4
Number of patients in the safety evaluable population

| | | | | | N(EUR |
|---|---|---|---|---|---|
| | Arm | N | N | N | genetics |
| Trial | Abbreviation | (safety) | (consent) | (EUR) | QC) |
| IMvigor211 | Atezo | 459 | 243 | 228 | 224 |
| IMvigor211 | Chemo | 443 | 236 | 212 | 205 |
| IMpower150 | ABCP | 393 | 249 | 210 | 200 |
| IMpower150 | ACP | 400 | 267 | 229 | 223 |
| IMpower150 | BCP | 394 | 236 | 185 | 178 |
| IMpower131 | ACNabP | 334 | 192 | 161 | 155 |
| IMpower131 | ACP | 332 | 188 | 165 | 158 |
| IMpower131 | CNabP | 334 | 193 | 160 | 155 |
| IMpower130 | ACNabP | 473 | 226 | 204 | 200 |
| IMpower130 | CNabP | 232 | 116 | 103 | 103 |
| IMpower133 | ACE | 250 | 96 | 77 | 76 |
| IMpower133 | CE | 244 | 85 | 71 | 71 |
| IMpassion130 | ANabP | 460 | 259 | 166 | 145 |
| IMpassion130 | NabP | 430 | 230 | 142 | 132 |
| IMmotion151 | AB | 451 | 252 | 214 | 203 |
| IMmotion151 | SUN | 446 | 218 | 194 | 189 |

Number of patients that provided informed consent for genetic data collection, were of European (EUR) ancestry, and met genotype and population QC filters separated by trial and arm.
Abbreviations: Atezo = atezolizumab monotherapy; A = atezolizumab; C = carboplatin; P = paclitaxel; NabP = Nab-paclitaxel; B = bevacizumab; SUN = sunitinib; E = etoposide; Chemo = taxanes or vinflunine; N(EUR) designates individuals with ADMIXTURE EUR ancestry coefficients of >0.7, N(EUR QC) designates individuals kept after population and genotype level QC filters.

i. Endocrinopathies Analyzed

irAEs were defined on the basis of an adverse events of special interest (AESI) strategy uniformly applied across studies. The original study protocols cited in Table 3 provide details on this methodology. The present study focused on the following endocrinopathies that were captured by this methodology: hypothyroidism, hyperthyroidism, adrenal insufficiency, type 1 diabetes mellitus, and hypophysitis. Patients with active autoimmune disease were excluded from the clinical trials in accordance with each study protocol.

ii. Frequency of Thyroid Function Lab Testing

In all studies, endocrine irAEs were captured on the basis of clinical symptoms and investigator lab tests as they appeared during treatment. Study protocols included provisions for thyroid hormone testing at regular intervals during treatment that varied on a per study basis. IMvigor211 initially conducted lab tests for thyroid-stimulating hormone (TSH), free thyroxine (fT4), and free triiodothyronine (fT3) levels at screening and treatment discontinuation, but a protocol amendment changed this to monitor at cycle 5 and every 4 cycles thereafter. The IMpower studies measured TSH, fT4, and fT3 levels at screening, treatment discontinuation, Cycle 1, and every fourth cycle thereafter. IMmotion151 and IMpassion130 conducted thyroid labs at screening, treatment discontinuation, and every two cycles (starting at Cycle 2).

iii. Results

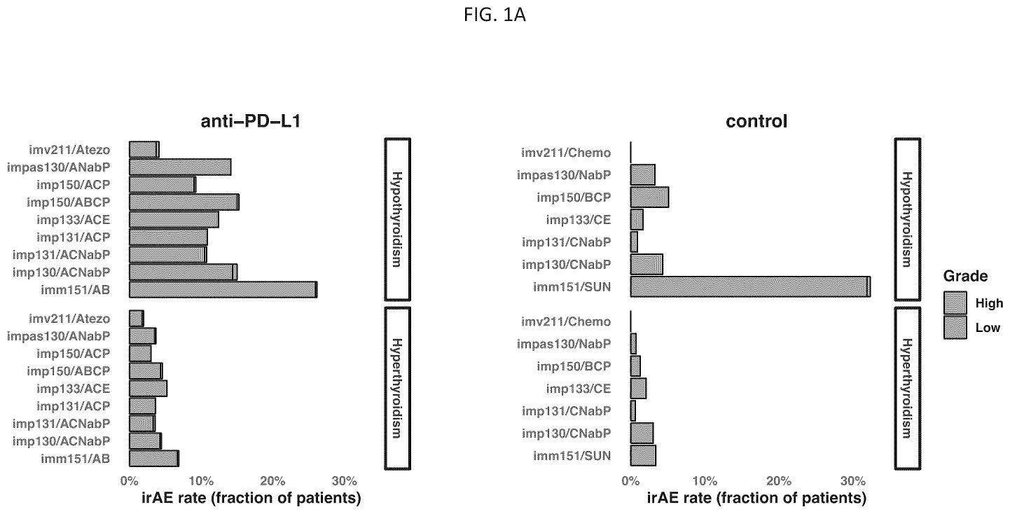

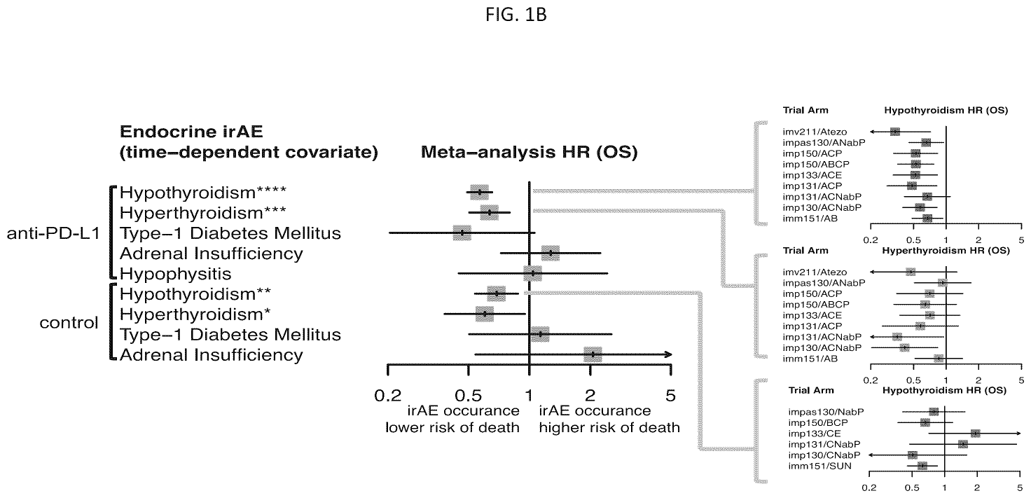

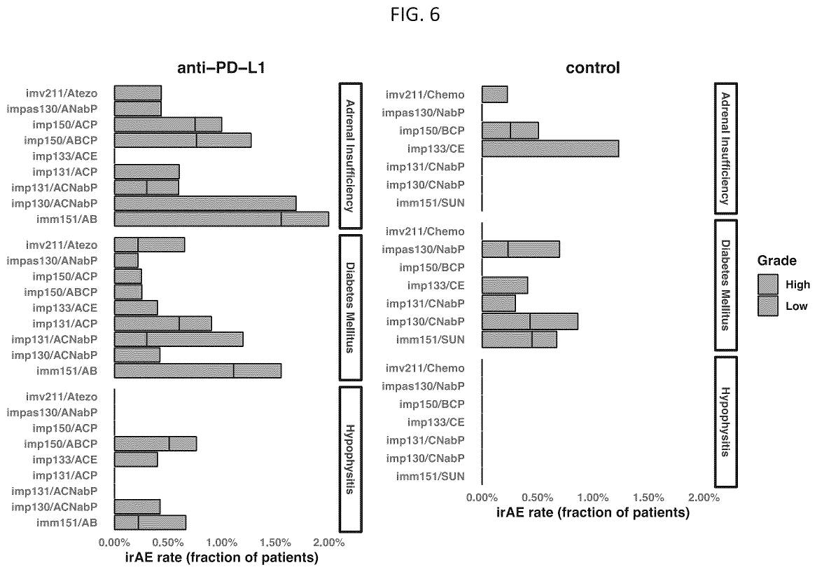

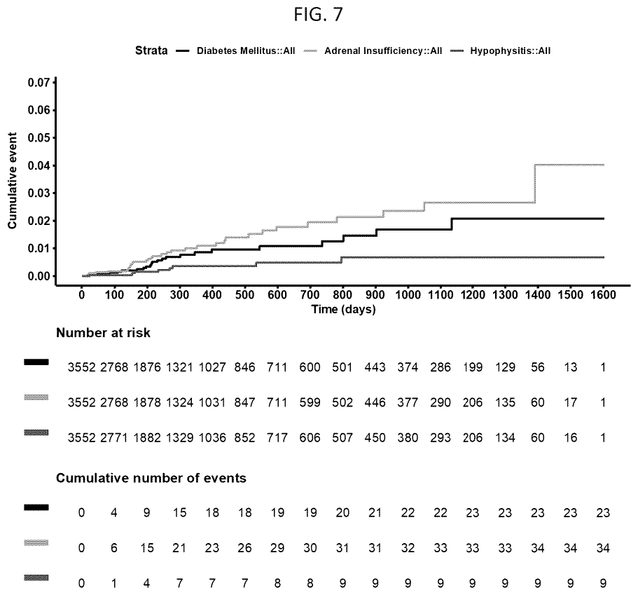

A total of 6,075 patients were in the safety evaluable population in these trials (Table 4), of which N=3,552 received atezolizumab in combination or as monotherapy. Consistent with prior reporting, adrenal insufficiency, type-1 diabetes mellitus (T1D), and hypophysitis were rare, occurring in about 0-0.17% of atezolizumab treated patients, and the probability of occurrence of these events was significantly less than 2% after 1.5 years from the start of treatment (FIGS. 6 and 7). In contrast, thyroid dysfunction, both hypothyroidism and hyperthyroidism, were more common (occurring in 3-26% of patients), consistent with results reported for other PD-1 checkpoint inhibitors (FIG. 1A) (Barroso-Sousa et al., JAMA Oncol, 4(2): 173-182, 2018).

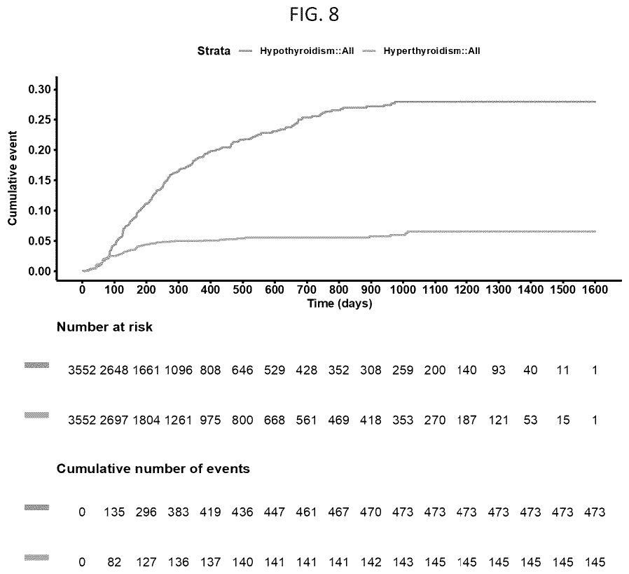

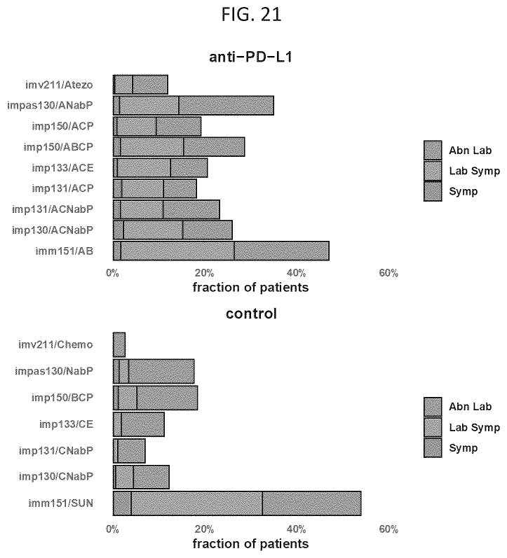

To assess the accuracy with which thyroid irAEs were identified in these trials, patient thyroid stimulating hormone (TSH) lab measurements were examined over the course of treatment. The fraction of patients with symptomatic hypothyroidism irAEs were a subset of those that experienced abnormal TSH level (>5 mU/L) events, and yet, nearly all symptomatic events were preceded, within 7 days, by an abnormal TSH measurement (FIG. 21). It was additionally considered that inflammation of the thyroid can initially appear as hyperthyroidism eventually leading to hypothyroidism as thyroid function is impaired (Chaker et al., Lancet, 390(10101): 1550-1562, 2017). The present study assessed whether such a temporal pattern was present in anti-PD-L1 treated patients by using the time at which these events were observed after the start of treatment. Combining all the patients treated with atezolizumab, the plateau in cumulative event probability occurred earlier for hyperthyroidism than for hypothyroidism (FIG. 8). Of the 473 atezolizumab treated patients that developed hypothyroidism, 70 were identified for whom both thyroid dysfunction events were reported. In 85.7% (60/70) of these patients, hyperthyroidism was diagnosed before hypothyroidism. Overall, this analysis indicates that the thyroid irAE data were consistent with expectations of their clinical course.

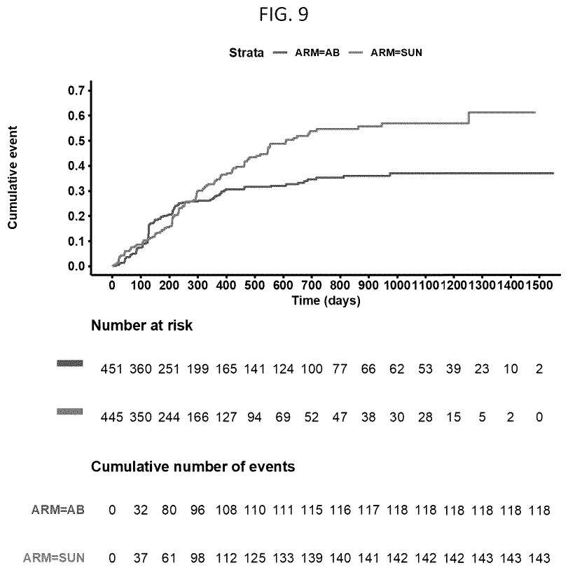

Treatment with atezolizumab as a monotherapy or in combination was associated with increased risk of hypothyroidism and hyperthyroidism as compared to patients receiving chemotherapies (FIG. 1A). An exception was the sunitinib arm of IMmotion151, which accounted for 74% (144/195) of the hypothyroidism events in the control arms. Sunitinib is a tyrosine kinase inhibitor that is also known to induce hypothyroidism in treated patients (Schmidinger et al., Cancer, 117(3): 534-544, 2011). Because of the use of sunitinib in the control arm, thyroid function was monitored at a higher frequency in IMmotion151, possibly accounting for the higher fraction of hypothyroid patients that were identified in both arms. Risk of sunitinib-induced hypothyroidism increased with the duration of treatment, a pattern not observed in atezolizumab-treated patients as the probability of developing hypothyroidism after 500 days from the start of treatment was significantly higher in sunitinib-treated patients than in patients treated with the atezolizumab combination (FIG. 9). It was estimated that patients were 2.49 (95% CI 1.72-3.60) times more likely to develop hyperthyroidism and, excluding IMmotion151, 3.77 (95% CI 2.39-5.93) times more likely to develop hypothyroidism during treatment with atezolizumab as a monotherapy or in combination as compared to the control arms.

### Example 2. Thyroid Dysfunction is Associated with Longer Overall Survival (OS)

Prior studies have observed associations between endocrine irAEs and longer patient survival during monotherapy treatment with the PD-1 checkpoint inhibitors pembrolizumab and nivolumab ((Kotwal et al., Thyroid, 30: 177-184, 2020; Osorio et al., Ann. Oncol., 28: 583-589, 2017; Byun et al., Nat. Rev. Endocrinol., 13: 195-207, 2017)). However, not all of these studies considered that irAEs occur past the point of randomization, introducing the potential for survival bias. Additionally, whether this association holds when PD-1 checkpoint inhibitors are combined with chemotherapies has not been tested.

The present study used a time-dependent covariate in a Cox proportional hazards model to address survivor bias, as described below.

i. Time-Dependent Covariate to Overall Survival Association Meta-Analysis

A time-dependent covariate was used to assess whether there exists an association between occurrence of an irAE and overall survival (OS). The time-dependent covariate was set to zero before the irAE onset and then to one after the irAE to estimate the association between the occurrence of irAEs and OS. The covariate was set to zero for patients that did not experience the irAE. The time-dependent covariate was constructed using the tmerge function in the survival package in R. This generated interval data for use in a Cox proportional hazards model. Across the trial arms, an individual participant data meta-analysis was then performed using the coxme package in R. The model used a differing baseline hazard for each trial arm, and also allowed for heterogeneity of the hazard ratio across the trial arms by use of a random effect term as indicated by the following R formula: Surv(tstart, tstop, OS.tv) ˜irAE+(irAEltrial.arm)+strata(trial.arm), where irAE corresponds to the time dependent covariate, trial.arm stratifies and groups according to trial arm, and tstart and tstop designate an interval, and OS.tv is set to 1 if a death event occurred at the end of the interval and 0 if censored or if the interval preceded the irAE.

ii. Results

Both hypothyroidism (meta-analysis p=5.26×10⁻¹⁵, HR=0.57, 95% CI 0.49-0.65) and hyperthyroidism (meta-analysis p=1.02×10⁻⁴, HR=0.63, 95% CI 0.50-0.79) were found to be associated with longer overall survival (OS) during atezolizumab treatment both as monotherapy and in combination with chemotherapies (FIG. 1B). This was confirmed by landmark analysis, as hypothyroidism irAEs occurring in the first 5 months (p=0.0024, HR=0.74, 95% CI 0.56-0.99) were also associated with longer OS. No association between OS and other endocrinopathies, including type-1 diabetes mellitus (p=0.067, HR=0.46, 95% CI 0.20-1.05), adrenal insufficiency (p=0.40, HR=1.27, 95% CI 0.72-2.24), and hypophysitis (p=0.92, HR=1.04, 95% CI 0.45-2.42), was observed. As these events were rare (FIG. 6), statistical power to detect a general association with endocrinopathies and OS was limited. The 95% confidence interval suggests an association may additionally exist between type-1 diabetes in atezolizumab treated patients and longer OS (FIG. 1B). Last, confirming prior studies, but also accounting for survival bias, a weaker association between OS and hypothyroidism was found in patients undergoing sunitinib treatment (p=0.0039, HR=0.62, 95% CI 0.45-0.85) (Schmidinger et al., Cancer, 117: 534-544, 2011).

### Example 3. Shared Genetic Etiology Between Hypothyroidism and Thyroid irAEs

Hypothyroidism also occurs in human populations in otherwise healthy individuals, e.g., individuals not receiving immune checkpoint inhibitors, bevacizumab, or chemotherapies, over the course of a lifetime due to environmental exposures and genetic factors. Hypothyroidism has a population prevalence estimated to be about 5% in Europeans (Chaker et al., Lancet, 390(10101): 1550-1562, 2017). Given its prevalence, GWAS have previously been conducted to identify loci associated with lifetime risk. These studies have confirmed that the majority of hypothyroidism in Europeans is caused by autoimmunity, as many of the loci are found near genes involved in immune regulation and in GWAS of other autoimmune diseases (Eriksson et al., PLoS One, 7: e34442, 2012). One of the largest of these GWAS used data from the UK Biobank (Kichaev et al., Am. J. Hum. Genet., 104: 65-75, 2019). This GWAS of 25,072 self-reported and International Statistical Classification of Diseases and Related Health Problems (ICD) code diagnosed cases and 383,887 controls from the UK Biobank was repeated using SAIGE, a method that better controls type-1 error in case control imbalanced GWAS (Table 1), as described below (Zhou et al., Nat. Genet., 50: 1335-1341, 2018).

i. Construction of a UKBiobank LD Reference Panel

Individuals that were not used in the principal component analysis (PCA) calculation, were outliers for heterozygosity or missing rate, had excess (ten or more) relatives identified, or had evidence of sex chromosome aneuploidy were removed. Individuals that self-reported “British,” “Irish,” “White,” or “Any other white background” ancestry were then selected. 10,000 individuals were randomly sampled from this cohort to construct a linkage disequilibrium (LD) reference panel. The per variant minor allele frequency (MAF) and imputation INFO scores in this cohort were recomputed, and variants with MAF<0.001 were removed. Variants meeting the following criterial for INFO score at several levels of MAF were kept: Info>0.3 for MAF>0.03; Info>0.6 for MAF 0.01-0.03; Info>0.8 for MAF 0.005-0.01; Info>0.9 for MAF 0.001-0.005. These filters left a total of 12.5 million variants in the LD reference panel. This LD reference panel was used in all subsequent analyses as a European LD reference panel.

ii. Fine Mapping

For fine mapping, an approach used by the DIAGRAM consortium in type-2 diabetes (T2D) (Mahajan et al., Nat. Genet., 50: 1505-1513, 2018) was adapted. As detailed below, we used genome-wide complex trait analysis-conditional and joint analysis (GCTA-COJO) to perform forward selection using approximate conditional analyses to detect distinct association signals (Yang et al., Nat. Genet., 44: 369-375, S1, 2012). GCTA-COJO performs approximate conditional analysis using genome-wide association study (GWAS) summary statistics and LD reference panel. The following algorithm was used to identify a set of independent signals, represented by a set of conditioning single-nucleotide polymorphisms (SNPs), within a given associated locus:
1. Identify a lead SNP from the unconditional meta-analysis and define locus ±500 kb around the lead SNP. Merge overlapping loci into one locus.
2. Condition out the lead SNP using GCTA-COJO.
3. If top SNP after conditional meta-analysis meets the genome-wide significance threshold (5×10⁻⁸) then repeat step 2, conditioning out the original lead SNP and the new top SNP.
4. Continue steps 2 and 3—effectively forward selection—until the residual conditional meta-analysis does not meet significance threshold.
5. This procedure will provide a set of “conditioning SNPs” at a given locus.

“Wakefield” credible sets were then constructed from the independent signals represented by a set of conditioning SNPs (Wakefield et al., Am, J. Hum. Genet., 81: 208-227, 2007). The algorithm conditioned out all but one SNP to obtain conditionally independent signals and corresponding summary statistics, which were then used to construct credible sets. To illustrate the algorithm for constructing credible sets, an example for three conditioning SNPs is provided: rsA, rsB and rsC.
1. To get the conditional association signal for rsA, condition out both rsB and rsC using GCTA-COJO.
2. Using the resulting conditional summary stats, compute the per variant approximate Bayes factor (ABF), where β corresponds to the log odds ratio and 6 the standard error after conditioning out all but the focal variant.

$$r = \frac{W}{W + {σ}^{2}}$$

$$z = \frac{β}{σ}$$

$$A B {F}_{{H}_{0}} = \frac{1}{\sqrt{1 - r}} exp \{ - \frac{{z}^{2}}{2} r \}$$

$$A B F = \frac{1}{A B {F}_{{H}_{0}}}$$

Here, W is set to W=0.04, which assumes that the 97.5% point of the prior is 1.48, that is, the prior probability that the odds ratio is greater than the 97.5% point is 0.025.
3. Compute the posterior probably of association (PPA) values for variant i by normalizing by all variants in the region by the sum of the ABF values

$${PPA}_{i} = \frac{A B {F}_{i}}{{\sum }_{k} A B {F}_{k}}$$

Construct the 99% Wakefield credible set was by ordering all variants in descending order of their PPA including the ordered variants into the set until the cumulative PPA is ≥0.99.
4. Repeat same process to obtain association signal for rsB by conditioning out rsA and rsC, and association signal for rsC by conditioning out rsA and rsB. After each conditioning step, compute the PPA and Wakefield credible steps.

This algorithm was applied to the hypothyroidism, T1D, and vitiligo summary statistics. Variants with the highest PPA within each credible set and their corresponding conditional effect sizes were used to compute a polygenic risk score as detailed in Example 4.

iii. Construction of an LDpred2-Auto Hypothyroidism PRS

After the harmonization steps above, the number of variants was restricted to those in the HapMap3 project on the basis of rsid match to a UK Biobank provided rsid. In total, 1,099,649 HapMap3 variants were used. Following the methods detailed in the LDpred2 vignette, genomic positions (in bp) were interpolated to genetic positions (in cM). Next, the correlation matrices were computed chromosome-wise using a window size of 3 cM. LDpred2-auto running 24 separate Gibb's samplers was then applied to the following initial values for the fraction of causal variants p: 1e-04, 1e-04, 2e-04, 3e-04, 5e-04, 7e-04, 0.001, 0.0015, 0.0022, 0.0032, 0.0047, 0.0069, 0.0101, 0.0149, 0.0219, 0.0322, 0.0473, 0.0695, 0.1021, 0.1501, 0.2206, 0.3241, 0.4763, 0.7. The initial value for heritability h² was set to the per-chromosome estimate of heritability obtained by LD-score regression. Each run of LDpred2-auto Gibb's sampler generated a per-chromosome estimated value for h² and p as well as set of shrunk beta coefficients. The paths of the Gibb's samplers for p and h² were inspected, and we found that the majority of Gibb's samplers converged to the same values for these parameters. Deviating from the recommendations of the vignette, the median value of the estimated fraction of causal variants p was computed across all the 24 Gibb's samplers. The shrunk beta coefficients associated to this median value for p were selected. This approach was applied chromosome-wise. Last, the selected shrunk beta coefficients were combined genome-wide and the PRS was computed across 1,099,649 HapMap variants with these coefficients within the atezolizumab clinical trial cohort as described in the previous section.

iv. GWAS Results

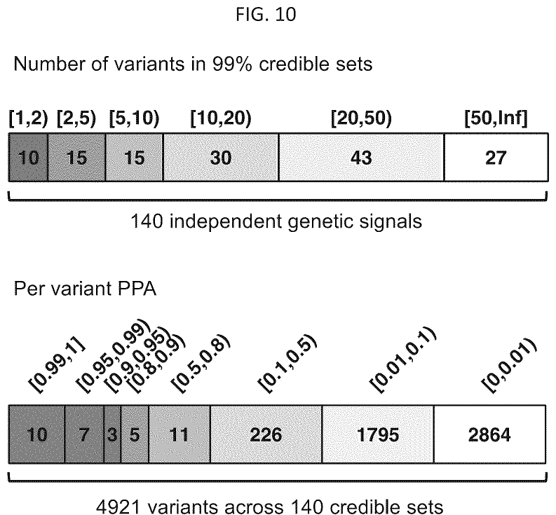

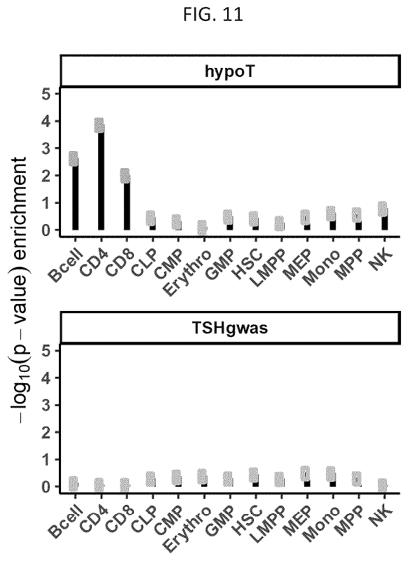

140 independent genome-wide significant signals in the GWAS were identified, and credible sets were computed which accounted for 99% of the posterior probability of association (PPA) for a given signal (Table 5; FIG. 10). Using LD score regression, it was estimated that the heritability captured by this GWAS was 13.1% (Finucane et al., Nat. Genet., 47: 1228-1235, 2015). Heritability of hypothyroidism risk was enriched in cell type-specific accessible chromatin, as measured by ATAC-seq in CD4⁺ T-cells, CD8⁺ T-cells, and B-cells (FIG. 11), reflecting the contribution of immune cells involved in autoimmunity. ATAC-seq data was from Corces et al., 2016.

TABLE 5
All variants in 99% credible sets used in PRS for hypothyroidism

| | Conditionally | | |
|---|---|---|---|
| | Independent | Non-Effect | |
| Variant ID | log-odds ratio | Allele | Effect Allele |
| rs10748781 | −0.0645174 | C | A |
| rs76169968 | −0.1175149 | G | A |
| rs10742340 | 0.09236397 | T | C |
| rs7936397 | −0.0717082 | G | A |
| rs97384 | 0.06942892 | T | C |
| rs479777 | −0.0647329 | T | C |
| rs7971751 | −0.0712043 | A | C |
| rs10844682 | −0.067431 | T | C |
| rs767474411 | 0.0891158 | CAGGCAGTG | C |
| rs35407628 | −0.0788206 | G | T |
| rs7333647 | 0.0624215 | T | C |
| rs2324919 | 0.07212558 | C | T |
| rs76247422 | 0.07890462 | C | T |
| rs7173565 | 0.09306097 | T | C |
| rs4494538 | −0.0792404 | C | T |
| rs8054578 | −0.0723474 | A | G |
| rs4794063 | 0.08561668 | C | T |
| 17:8843722_CA_C | 0.08385823 | CA | C |
| rs2847297 | 0.07425612 | A | G |
| rs1562722 | 0.06693033 | A | G |
| rs12973608 | 0.07890282 | A | C |
| 1:108362169_GAC_G | 0.24020883 | GAC | G |
| rs926103 | −0.0752853 | T | C |
| rs1617333 | 0.06189822 | A | G |
| rs56255908 | 0.08844664 | C | T |
| rs12117927 | 0.06045137 | C | A |
| rs671565 | −0.0564148 | G | A |
| rs4320727 | 0.05952445 | G | A |
| rs2234167 | 0.08811559 | G | A |
| rs573741 | 0.10032956 | C | A |
| rs301799 | −0.0613473 | C | T |
| rs4820437 | −0.0728355 | T | C |
| rs7574865 | −0.150298 | T | G |
| rs12623702 | 0.05817116 | A | G |
| 2:55855589_CTATT_C | 0.13915637 | CTATT | C |
| rs5865 | −0.0591073 | C | T |
| rs62261536 | −0.0886917 | A | C |
| 3:105935232_TA_T | 0.107607 | TA | T |
| rs4688009 | 0.07987748 | C | T |
| rs767312898 | 0.07957335 | C | CT |
| rs114558062 | 0.24646 | T | C |
| rs4647214 | −0.057454 | A | AT |
| rs11706384 | 0.0795633 | G | T |
| rs35836357 | 0.08629086 | C | CT |
| rs1991797 | −0.075499 | G | T |
| rs2434973 | 0.08688655 | T | C |
| rs7753634 | 0.074868 | G | A |
| rs544188579 | 0.688373 | A | G |
| rs6904596 | 0.150504 | G | A |
| rs2229637 | −0.0650904 | G | A |
| rs6457834 | 0.07186521 | C | A |
| rs221781 | 0.09649662 | A | G |
| rs11765986 | 0.06271961 | C | T |
| rs2445610 | −0.0863555 | A | G |
| rs759649 | −0.0611995 | G | A |
| rs1561924 | −0.0922268 | G | A |
| rs2921071 | −0.0901845 | A | C |
| 9:110602043_AC_A | 0.13021748 | AC | A |
| rs1107342 | 0.06141108 | T | C |
| rs9697210 | −0.0820141 | G | A |
| rs2123340 | −0.0822999 | G | A |
| rs34334084 | −0.1453418 | A | AT |
| rs367023 | −0.0690278 | A | G |
| rs7437047 | 0.07849538 | G | A |
| rs4444866 | −0.0711004 | C | T |
| rs6833591 | −0.0659346 | A | G |
| rs714027 | 0.08078938 | A | G |
| rs10036386 | 0.0707017 | C | T |
| rs12697352 | −0.0714671 | G | A |
| rs7905731 | −0.084119 | T | C |
| rs11783023 | −0.0639456 | C | T |
| rs7005834 | −0.0684017 | C | T |
| rs1032129 | −0.0658646 | A | C |
| rs2208397 | 0.07161724 | T | G |
| rs143627643 | 0.06255622 | T | TA |
| rs11893621 | 0.05788304 | T | C |
| rs10277273 | −0.0617991 | T | G |
| rs202157095 | 0.11612038 | T | TGAAAAGG |
| rs1079418 | −0.0650157 | A | G |
| rs7248104 | −0.0554714 | G | A |
| rs2403967 | −0.0730613 | A | C |
| rs10424978 | −0.0904996 | C | A |
| rs34678053 | −0.0950411 | G | A |
| rs2223385 | 0.0668503 | G | A |
| rs10917477 | 0.06034776 | A | G |
| rs187855607 | 0.354829 | T | C |
| rs3778607 | 0.07015064 | A | G |
| rs113229608 | 0.1328165 | C | A |
| rs34046593 | 0.08433629 | G | A |
| rs9511151 | −0.0686196 | G | A |
| rs925489 | 0.21786548 | C | T |
| rs947474 | 0.0876878 | G | A |
| rs911760 | 0.09495535 | C | A |
| rs810048 | −0.1083316 | C | T |
| rs10930013 | 0.05900174 | G | A |
| rs72928038 | 0.0947767 | G | A |
| rs150332089 | 0.08746645 | C | A |
| rs12325861 | 0.0926592 | T | C |
| rs11085727 | −0.0705869 | C | T |
| rs484959 | 0.06211277 | T | C |
| rs969256 | 0.07329344 | C | A |
| rs1724088 | 0.06635891 | G | A |
| rs745592798 | 0.355644 | A | AT |
| rs9366078 | −0.124017 | A | G |
| rs113473633 | −0.1942729 | A | G |
| rs35144535 | 0.08841282 | A | G |
| rs114378220 | 0.12409499 | C | T |
| rs61776678 | −0.0678625 | G | A |
| rs62182595 | −0.0993243 | G | A |
| rs7090530 | 0.11163 | C | A |
| rs693939 | −0.0638766 | C | T |
| rs11675342 | 0.10280562 | C | T |
| rs6780858 | −0.138311 | A | G |
| rs12582330 | −0.0866199 | G | T |
| rs12722518 | 0.206676 | A | C |
| rs7758816 | −0.0604311 | T | C |
| rs1790963 | −0.0634494 | C | A |
| rs12980063 | −0.0763596 | A | G |
| rs6926462 | 0.0596364 | G | A |
| rs78534766 | 0.469271 | C | A |
| rs78847176 | 0.373591 | C | T |
| rs1534430 | −0.091249 | C | T |
| rs1810396 | −0.082111 | A | G |
| rs2111485 | 0.0842816 | A | G |
| rs654537 | 0.0899576 | G | A |
| rs229540 | 0.1166661 | T | G |
| rs231779 | 0.194651 | C | T |
| rs244674 | −0.1219735 | T | C |
| rs2476601 | −0.43915 | A | G |
| rs2745803 | −0.0876161 | A | G |
| rs3184504 | −0.2192079 | T | C |
| rs34916596 | −0.0619239 | T | TA |
| rs61759532 | 0.0971685 | C | T |
| rs3775291 | −0.0804566 | C | T |
| rs4409785 | 0.15849194 | T | C |
| rs9497965 | 0.0768222 | C | T |
| rs71508903 | 0.140487 | C | T |
| rs72871627 | −0.259976 | A | G |
| rs76428106 | 0.57214634 | T | C |
| rs78306789 | 0.13951 | G | A |

### Example 4. Polygenic Risk Scores

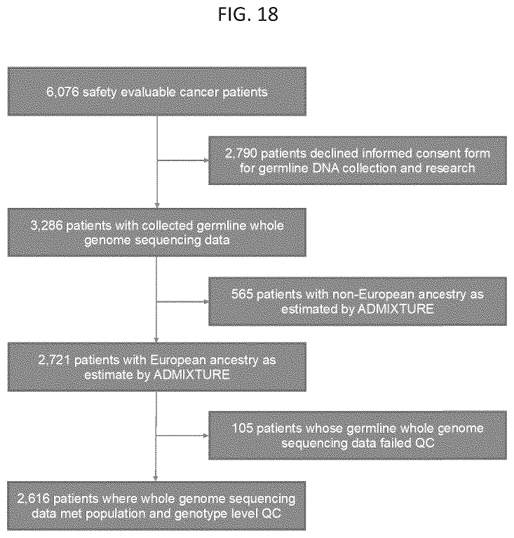

It was next investigated whether genetic variants that affect lifetime risk of hypothyroidism also contribute to risk of thyroid irAEs during the shorter duration of atezolizumab treatment. This hypothesis was tested by constructing a PRS using 140 variants with the highest PPA for each of the genetic credible sets obtained by fine mapping. 30×whole genome sequencing data were collected and analyzed from 2,616 patients, of which 1,584 were treated with atezolizumab in combination or as monotherapy, provided informed consent for genetic data collection, were of European ancestry (to match the ancestry of the population used to construct the PRS), and met sample and population quality control filters (Table 4; FIG. 18). The UK Biobank-derived hypothyroidism PRS was applied to time to irAE data from these patients.

i. Whole Genome Sequencing and Sample/Variant QC of Atezolizumab Trial Cohort

Genomic DNA was extracted from blood samples using the DNA Blood400 kit (CHEMAGIC™) and eluted in 50 μL Elution Buffer (EB, Qiagen). DNA was sheared using a LE220 Focused-ultrasonicator (Covarisg) and sequencing libraries were prepared using the TruSeq DNA Nano HT kit (Illumina). Libraries were sequenced at Human Longevity (San Diego, CA, USA). All sequencing data was checked for concordance with SNP fingerprint data collected before sequencing. 150 bp paired-end whole-genome sequencing (WGS) data was generated to an average read depth of 30×using the HiSeq platform (Illumina X10, San Diego, CA, USA).

Reads were aligned using the functionally equivalent BAM (FEB) pipeline. Samples were joint genotyped using the Sentieon genome analysis toolkit (GATK). Only variants flagged as PASS and genotype calls with GQ >20 were used. After application of the GQ filter, variants with genotype call missing rate of >0.1 were removed. Multi-allelic sites were handled by keeping only calls for the two most common alleles, all other calls were set to missing. Common variants with MAF>0.01 in this cohort were extracted. Samples were removed if they had a high within-sample missing rate of >0.1. Samples were then merged with 1000G samples, and LD pruned. ADMIXTURE v1.23 was used to estimate ancestry in the 5 major populations using supervised mode. Samples with >0.7 EUR ancestry were extracted and analyzed for heterozygosity outliers by estimating the per sample F inbreeding coefficient. EUR samples with an F statistic more than 5 standard deviations from the mean were removed. EUR samples were then analyzed for relatedness using the KING method implemented in the SNPRelate R package. Sample pairs with Pr(no allele shared), k0<0.4, were identified and the sample with the least missing variant calls was kept and the other removed. PCA was then performed using the implementation in the SNPRelate package. Five rounds of PCA outlier removal iterations were performed removing samples that were >6 standard deviations from the top 10 eigenvectors at each iteration. The final PCA was then performed to compute 5 eigenvectors that were subsequently used to account for any remaining population stratification.

Using this final EUR cohort with missing rate, heterozygosity, relatedness, and PCA outlier samples removed, variant level QC was performed. Heterozygous calls for variants were analyzed for evidence of allele imbalance by summing counts of each of the alleles at these het calls using the AD value in the VCF file and removing variants with an allele balance of <0.3 or >0.7 and a binomial test p<5×10⁻⁸. Variants were also analyzed for violation of Hardy Weinberg equilibrium at p<5×10⁻⁸ and any variants with MAF<0.001 were removed. In total, 14.3 million variants were left after these filtering steps.

ii. Harmonization of Summary Statistics with LD Reference Panel

Each of the summary statistics were harmonized with the LD reference panel as well as the atezolizumab trial whole genome sequencing data by using genomic position. The effect and non-effect alleles were also required to match between the LD reference, the atezolizumab trial data, and the summary statistics. Variants with strand ambiguity (A/T or C/G genotypes) were removed. The UCSC genome browser chain files were used to lift any hg19 coordinate systems to hg38 as necessary using the rtracklayer package in R. All variants in the MHC region (hg38, chr6: 28510120-33480577) were removed due to the complex LD in this region for both fine mapping and subsequently construction of a PRS.

iii. Construction and Computation of Polygenic Risk Scores

For each independent signal's 99% credible set, the variant with the highest PPA was used in the polygenic risk score. The score used effect sizes from the conditional summary statistics obtained after conditioning out all but one of the conditioning SNPs as detailed above. The PRS was computed as follows:

$$\hat{S} = {\sum }_{i = 1}^{M} {β}_{i} \cdot {G}_{i}$$

where M is the number of independent signals in the GWAS after fine mapping and βᵢ corresponds to the conditional effect size for the variant with the highest PPA for ith signal and Gᵢ={0,1,2} corresponds to the number of copies of the risk allele. PRSs were quantile normalized to the quantiles of a standard normal distribution to allow comparison across GWAS. While several recent methods have been developed to use all variants in the genome to compute PRSs with promising improvements in accuracy, analysis was limited to genome-wide significance signals to enable a lasso regression analysis as described below.

iv. PRS and irAE Meta-Analysis Across Trial Arms

As individual participant data across trials was available, a one-step approach was used to conduct a meta-analysis. For time to event data, a mixed effects Cox model was used that allowed for a differing baseline hazard per trial arm using the coxme package in R. The analysis also accounted 5 genotype eigenvectors to account for remaining population stratification. The meta-analysis p-value corresponded a test for a non-zero coefficient on the PRS term in the following coxme model:

Surv(irAE.time,irAE.occured)˜PRS+(PRSItrial.arm)+EV.1+EV.2+EV.3+EV.4+EV.5+strata(trial.arm).

By modeling a random effect on PRS, the model allowed for some heterogeneity among the trial arm-specific effect sizes. Each PRS was quantile normalized to a standard normal distribution to allow comparison across GWAS.

v. Correlation Between Hypothyroidism PRS and Risk of Drug-Induced Hypothyroidism

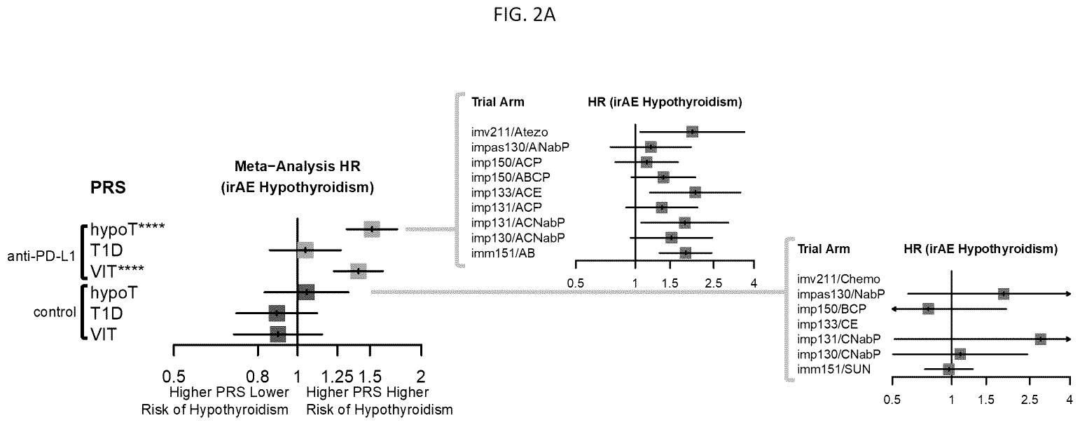

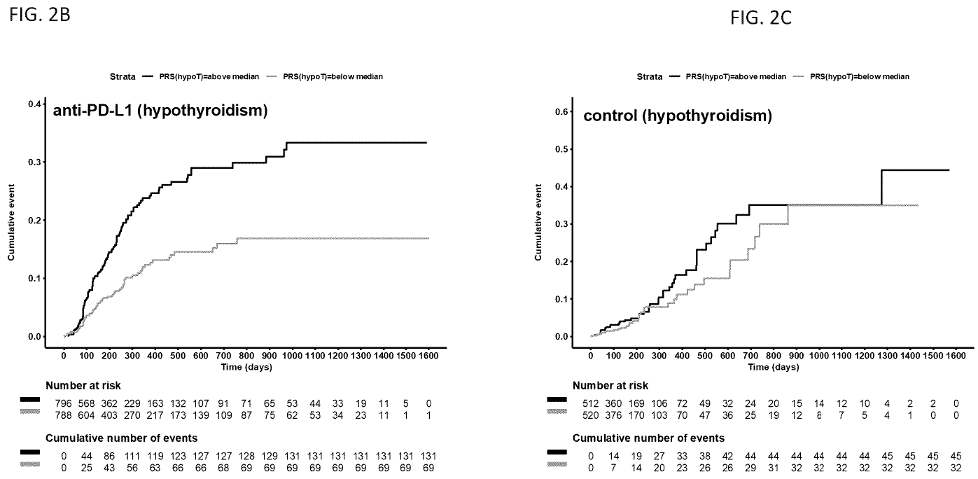

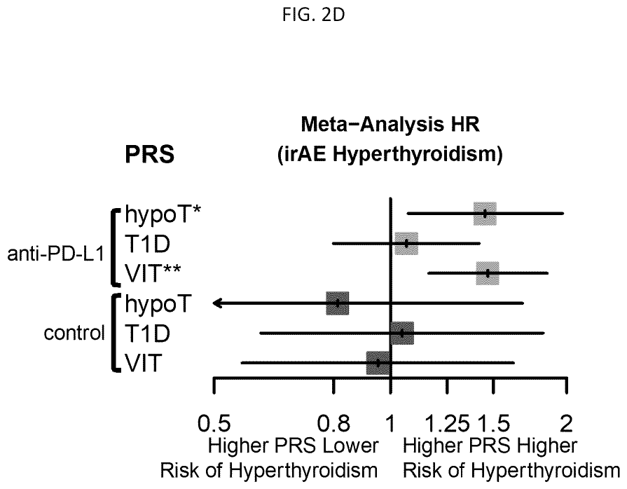

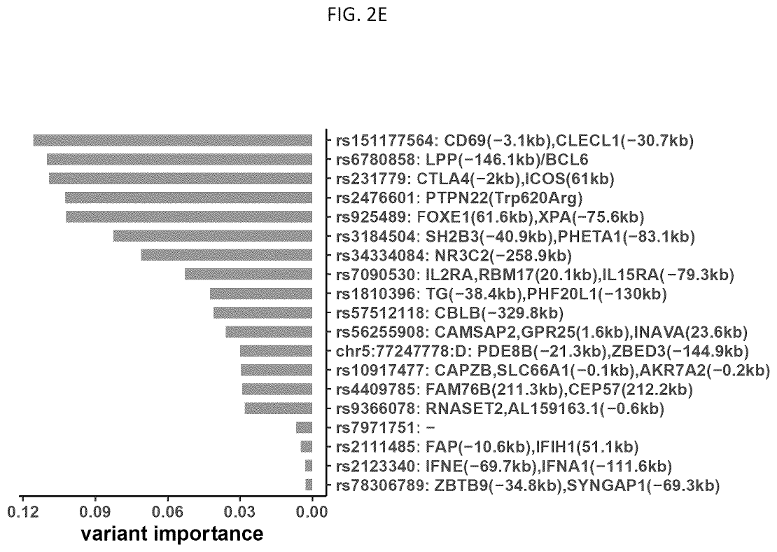

A higher PRS for hypothyroidism was associated with increased risk of hypothyroidism irAEs in the atezolizumab arms of the trials (meta-analysis, p=7.52×10⁻⁹; 95% CI HR=1.31-1.74 per unit normalized PRS). No association was found in the control arms (meta-analysis, p=0.67; HR=1.52, 95% CI 1.31-1.74 per unit normalized PRS), including the sunitinib arm of IMmotion151 (FIGS. 2A-2C). A higher hypothyroidism PRS was also associated with increased risk of hyperthyroidism, reflecting the temporal ordering of these irAEs (meta-analysis, p=0.016, 95% CI HR=1.07-1.96 per unit normalized PRS, FIG. 2D).

The robustness of the association between genetic variation and hypothyroidism irAEs in atezolizumab-treated patients was tested using several approaches. As the 140-variant PRS included only genome-wide significant signals, a second PRS was constructed that consisted of 1,099,649 HapMap3 variants using LDpred2-auto, a method that relies on beta-shrinkage to incorporate signals below genome-wide significance (Prive et al., Bioinformatics, 36 (22-23): 5424-5431, 2020). Using the LDpred2-auto PRS, it was confirmed that the finding was robust to the underlying method for PRS construction (meta-analysis p=5.49×10⁻⁹, HR=1.49, 95% CI 1.30-1.71 per unit normalized PRS). It was additionally confirmed that the hypothyroidism PRS was associated with a broader event class, time to first abnormal TSH lab measurement (>5 mU/L) (meta-analysis, p=1.31×10⁻⁹, HR=1.40, 95% CI 1.25-1.56 per unit normalized PRS), in atezolizumab-treated patients. Across trials, the association was not correlated with the frequency of thyroid lab testing (FIG. 2A). Reflecting the temporal ordering of hyperthyroidism before hypothyroidism irAEs, it was found that a higher hypothyroidism PRS was associated with increased risk of hyperthyroidism (meta-analysis, p=0.016, 95% CI HR=1.07-1.96 per unit normalized PRS, FIG. 2C). Thus, shared genetic factors contribute to risk of atezolizumab induced hypothyroidism irAEs, which occur during cancer treatment, and to risk of hypothyroidism that occurs over a lifetime in European populations.

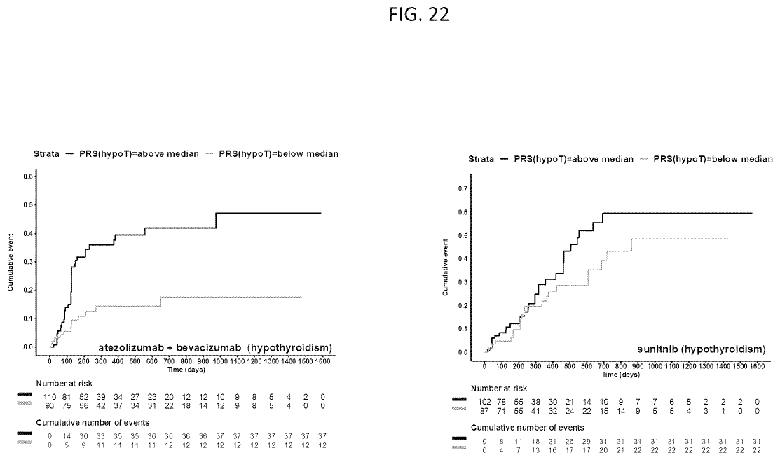

Patients in the control arms received combinations of chemotherapies, bevacizumab, or sunitinib reflecting standard of care for the cancer indication in which the trial was conducted. In contrast to atezolizumab, which targets the immune system, the drugs in the control arms act to induce death in proliferating cells, inhibit angiogenesis, or both. Combining all of the control arms, no association between the PRS and risk of hypothyroidism was found (meta-analysis, p=0.67; HR=1.05, 95% CI 0.83-1.11 per unit normalized PRS), indicating that genetic variation affecting the immune system did not alter risk of this event during treatment with these drugs (FIGS. 2A and 2B). The majority of the hypothyroidism events (53/77) in European patients with genetic data from the control arms occurred in the sunitinib arm of IMmotion151. It was separately confirmed that the hypothyroidism PRS was not associated with risk of sunitinib-induced hypothyroidism (FIGS. 2A and 22). The proportion of patients that developed hypothyroidism during sunitinib treatment was significantly higher than population prevalence, and prior studies have observed high rates of sunitinib-induced hypothyroidism in cancer patients (Schmidinger et al., Cancer, 117(3): 534-544, 2011). These observations indicate that the absence of an association with PRS sunitinib-induced hypothyroidism can be explained by its differing mechanism of action-receptor tyrosine kinase inhibition—as compared to atezolizumab.

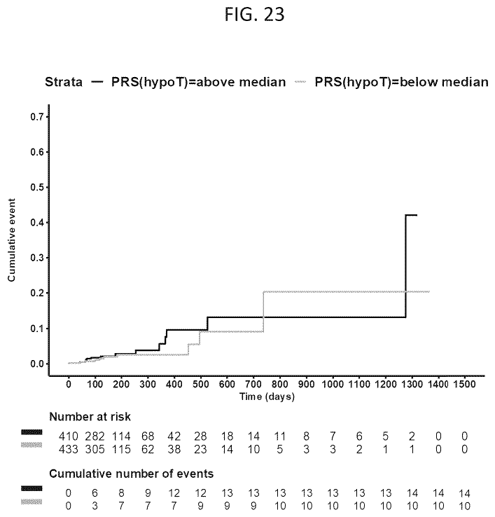

Excluding sunitinib, 24 hypothyroidism events were observed in 843 European chemotherapy-treated cancer patients with genetic data in the control arms. Given that the proportion of hypothyroidism events was below the population prevalence and these chemotherapies have not been associated with high rates of hypothyroidism during treatment, these events were likely lifetime cases found due to elevated monitoring and lab testing as per the protocols of the trials. While one might expect the hypothyroidism PRS to be associated with these events, no evidence was found for such an association (FIG. 23), and no difference was found in the PRS values between hypothyroid and non-hypothyroid (t-test, p=0.24) chemotherapy-treated cancer patients. To determine whether this was due to lack of power, cross-validation was used to estimate the effect size of the hypothyroidism PRS between lifetime cases and controls in in the UK Biobank. The power to detect a difference between these 24 cases and 819 controls was found to be limited (power=0.4 at a significance level of 0.01). Furthermore, assuming a similar proportion of such cases in atezolizumab-treated patients, there was little or no power to detect a difference (power <0.01) at a significance level observed for the hypothyroidism PRS and irAE association. Taken together, this analysis indicates that a small fraction of lifetime hypothyroidism cases are found in the trials studied, but do not account for the association observed between hypothyroidism PRS and atezolizumab-induced hypothyroidism irAEs.

vi. T1D and vitiligo PRSs

Autoimmune disorders, including T1D and vitiligo, co-occur with thyroid dysfunction, including hyperthyroidism and hypothyroidism over the course of a lifetime (Jin et al., Nat Genet, 48(11): 1418-1424, 2016). GWAS have also been conducted for these disorders to identify risk loci shared with hypothyroidism. Therefore, we investigated whether genetic variation ascertained by these studies was also associated with risk of thyroid irAEs during cancer immunotherapy treatment. To test this, PRSs were constructed for T1D and vitiligo using a consistent methodology and summary statistics from the largest meta-analyses of these diseases conducted in European populations to date (Table 1). No association was observed between the T1D PRS and increased risk of hypothyroidism or hyperthyroidism irAEs (FIGS. 2A and 2D). A higher vitiligo PRS was associated with increased risk of hypothyroidism irAEs (meta-analysis, p=1.10×10⁻⁶; HR=1.41, 95% CI 1.22-1.61 per unit normalized PRS) and hyperthyroidism irAEs (meta-analysis, p=0.0012; HR=1.46, 95% CI 1.16-1.85 per unit normalized PRS) in patients treated with atezolizumab, but not in the control arms (FIGS. 2A and 2D). These results are consistent with the genetic correlation (r_{g}=0.37; p=5.57×10⁻¹¹) between vitiligo and hypothyroidism as computed by LD score regression and emphasize the role of the immune system and autoimmunity during thyroid irAEs.

vii. Identification of Important PRS Variants by Survival Lasso Regression

A PRS represents a weighted linear sum of genetic variants that contribute to lifetime risk. Given that the environmental exposures that trigger thyroid autoimmunity or vitiligo in European populations over a lifetime are more varied than cancer treatment with atezolizumab, it was hypothesized that only a subset of these lifetime risk variants contribute to risk for hypothyroidism irAEs in atezolizumab-treated patients.

In order to identify which variants contributed significantly to the association between PRSs and hypothyroidism irAEs, an N× P data matrix was created for i=1 . . . N atezolizumab treated patients and p=1 . . . P variants from the polygenic risk score. For a given individual i in the data matrix and variant p, the element of the data matrix was set to number of copies of the risk allele carried by individual i multiplied by the conditional effect size of variant p. Genotype eigenvectors were added as additional columns to the data matrix. This data matrix was then fit using a survival lasso model where the coefficients of the model were constrained to be ≥0 with the exception of the coefficients associated with the genotype eigenvectors. The genotype eigenvectors were also excluded from the l₁ penalty, allowing the model to adjust for these covariates independently. 3-fold cross validation was used to estimate the best l₁ penalty parameter in a survival lasso model for time of hypothyroidism irAE occurrence. Cross validation was run 100 times, and the average obtained across runs. This run average best l₁ penalty parameter was used to determined which variants were retained when a model was fit to all of the data. To estimate the relative importance of retained variants, the resulting non-zero lasso coefficients for the variants were multiplied by the conditional betas provided by fine mapping, thus providing re-weighted coefficients after the model was fit to the time to hypothyroidism irAE data. The resulting absolute values were normalized to sum to one. It was confirmed that this approach did not assign any non-zero coefficients to variants for patients in the control arms that developed hypothyroidism. Candidate genes within and near the credible sets were identified using transcription start sites of APPRIS principal isoforms in the GENCODE v32 gene annotations.

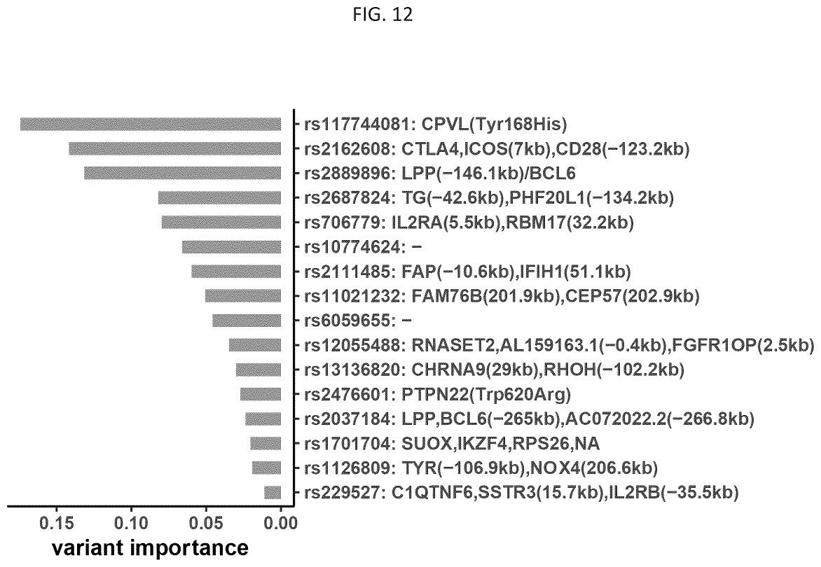

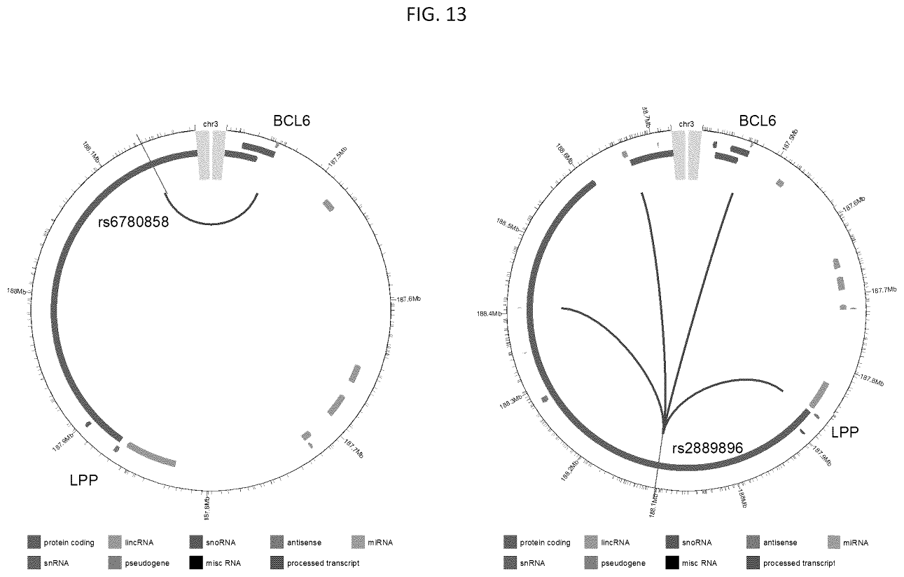

It was then determined which variants were retained when a model was fit to all of the data using the estimated sparseness penalty. 19 and 16 variants in the hypothyroidism and vitiligo PRSs, respectively, were assigned non-zero coefficients by lasso regression (FIG. 2E, FIG. 12). The transcription start sites (TSS) of genes within and near the credible sets (±500 kb from the ends) to which these variants belonged were examined (Tables 6-8). Genes implicated in immune regulation and autoimmunity, including CTLA4, CBLB, PTPN22, and 0069 as well as known autoantigens tyrosinase (TYR) and thyroglobulin (TG), were enriched nearthese retained variants. Lasso retained far fewer variants near genes implicated in thyroid development and function (e.g. FOXE1 and CAPZB). Variants near LPP, a gene with no known immune function, were also retained by lasso across both PRS. Further analysis of promoter capture Hi-C data and expression quantitative trait loci (eQTL) data indicated that these variants likely affect BCL6, the lineage-defining transcription factor for CD4⁺ T-follicular-helper (TFH) cells (Crotty et al., Immunity, 41(4): 529-542, 2014) (FIG. 13). The chord plot was generated using the Capture Hi-C Plotter, illustrating interactions with score ≥5 in the GM12878 lymphoblastoid cell line data set from Mifsud et al., Nat. Genet., 47: 598-606, 2015.

TABLE 6
Definitions of columns in Tables 7 and 8

| Column | Description |
|---|---|
| signal.lead | Variant id in UKBiobank of conditioning SNP identified |
| | by forward selection |
| n.variants | Number of variants in credible set |
| span.kb | Region spanned by credible set variants in kb |
| n.within | Number of TSSs within genomic region spanned by |
| | credible set |
| gene.within | Gene names of TSSs in region spanned by credible |
| | set; “—“ if >3 TSSs within region spanned by credible |
| | set |
| gene.dist | Top 2 closest genes in genomic distance from ends of |
| | the credible set. Gene name and distance in kb of TSS |
| | to credible set start or end is provided. “—“ if >3 |
| | TSSs within region spanned by credible set |

kb = kilo-bases; TSS = transcription start site

TABLE 7
Candidate genes within and near credible sets

| signal.lead | n.variants | span.kb | n.within | gene.within | gene.dist |
|---|---|---|---|---|---|
| rs2234167 | 64 | 242.4 | 3 | PRXL2B, | |
| | | | | MMEL1, | |
| | | | | TTC34 | |
| rs301799 | 148 | 395.1 | 0 | | SLC45A1(−27.8 kb), RERE(76.7 kb) |
| rs10917477 | 25 | 222.2 | 1 | CAPZB | SLC66A1(−0.1 kb), AKR7A2(−0.2 kb) |
| rs671565 | 88 | 236.4 | 8 | — | |
| rs4320727 | 38 | 542.7 | 4 | — | |
| rs61776678 | 14 | 306.8 | 5 | — | |
| rs573741 | 37 | 115.9 | 1 | JAK1 | JAK1(75 kb), RAVER2(−131.7 kb) |
| 1:108362169_GAC_G | 27 | 18.4 | 0 | | VAV3(132.5 kb), SLC25A24(367.7 kb) |
| rs484959 | 12 | 12.1 | 0 | | EPS8L3(−47.4 kb), GSTM3(−70.3 kb) |
| rs745592798 | 24 | 247.1 | 0 | | PHTF1(3.3 kb), RSBN1(56.6 kb) |
| rs2476601 | 2 | 73.8 | 1 | RSBN1 | PHTF1(−1.7 kb), PTPN22(36.8 kb) |
| rs810048 | 17 | 56.8 | 1 | CD2 | IGSF3(−52.4 kb), PTGFRN(132.9 kb) |
| rs926103 | 12 | 51.8 | 1 | SH2D2A | PRCC(−8.2 kb), HDGF(−24 kb) |
| rs1617333 | 21 | 30.9 | 0 | | CD247(51.5 kb), CREG1(86.7 kb) |
| rs56255908 | 66 | 158.4 | 1 | CAMSAP2 | GPR25(1.6 kb), INAVA(23.6 kb) |
| rs12117927 | 3 | 116.1 | 1 | EDARADD | LGALS8(52.4 kb), ERO1B(−67.7 kb) |
| rs11675342 | 2 | 9.6 | 0 | | TPO(0 kb), PXDN(331.1 kb) |
| rs367023 | 14 | 45 | 0 | | ID2(331.7 kb), KIDINS220(490.5 kb) |
| rs1534430 | 2 | 1.4 | 0 | | TRIB2(210.9 kb) |
| 2:55855589_CTATT_C | 42 | 105.8 | 1 | PPP4R3B | PNPT1(29.8 kb), CFAP36(−38.5 kb) |
| rs11893621 | 11 | 15.3 | 0 | | B3GNT2(−87.3 kb), TMEM17(188.2 kb) |
| rs5865 | 123 | 841.6 | 7 | — | |
| rs10930013 | 18 | 550.8 | 4 | — | |
| rs2111485 | 2 | 13.5 | 0 | | FAP(−10.6 kb), IFIH1(51.1 kb) |
| rs72871627 | 1 | 0 | 0 | | FAP(−37 kb), IFIH1(38.3 kb) |
| rs7574865 | 6 | 25.6 | 0 | | STAT4(46.4 kb), STAT1(−64.8 kb) |
| rs12623702 | 23 | 510.2 | 5 | — | |
| rs78847176 | 2 | 61.6 | 1 | CTLA4 | ICOS(13.9 kb), CD28(−154.6 kb) |
| rs62182595 | 8 | 10.5 | 1 | CTLA4 | ICOS(59.9 kb), CD28(−159.6 kb) |
| rs3087243 | 4 | 6 | 0 | | CTLA4(−2 kb), ICOS(61 kb) |
| rs150332089 | 13 | 26.6 | 1 | BHLHE40 | ARL8B(137.9 kb), EDEM1(203.4 kb) |
| rs767312898 | 136 | 283.5 | 2 | SYN2, TIMP4 | PPARG(9.2 kb), TAMM41(−147.9 kb) |
| rs4647214 | 212 | 333.6 | 4 | — | |
| rs11706384 | 14 | 122.6 | 2 | CX3CR1, | SLC25A38(11.3 kb), RPSA(34.7 kb) |
| | | | | CCR8 | |
| rs62261536 | 44 | 158.4 | 1 | CBLB | ALCAM(−363.3 kb) |
| rs13090803 | 51 | 53.1 | 0 | | CBLB(−329.8 kb) |
| rs969256 | 44 | 411.2 | 5 | — | |
| rs4688009 | 24 | 118.7 | 3 | TMEM39A, | |
| | | | | POGLUT1, | |
| | | | | TIMMDC1 | |
| rs113229608 | 24 | 94.8 | 2 | ILDR1, CD86 | CASR(73.7 kb), SLC15A2(−120.8 kb) |
| rs114558062 | 7 | 763 | 1 | LPP | BCL6(−177.9 kb), AC072022.2(−179.7 kb) |
| rs6780858 | 20 | 43.9 | 0 | | LPP(−146.1 kb) |
| rs7437047 | 30 | 24.9 | 0 | | CLNK(−15.6 kb), ZNF518B(−242.9 kb) |
| rs34046593 | 20 | 43.2 | 0 | | SMIM20(−169.7 kb), RBPJ(193.7 kb) |
| rs4444866 | 32 | 70.5 | 1 | CHRNA9 | RHOH(−87.3 kb), RBM47(161.2 kb) |
| rs113473633 | 2 | 37.2 | 0 | | NFKB1(−26.6 kb), SLC39A8(−182.5 kb) |
| rs6833591 | 48 | 386.2 | 3 | ADAD1, IL2, | |
| | | | | IL21 | |
| rs34334084 | 63 | 52.1 | 0 | | NR3C2(−258.9 kb) |
| rs35836357 | 161 | 121.4 | 1 | VEGFC | SPCS3(−378.1 kb), ASB5(−428.9 kb) |
| rs3775291 | 2 | 2.8 | 0 | | TLR3(−10.9 kb), FAM149A(21.5 kb) |
| rs12697352 | 15 | 77.9 | 1 | IL7R | CAPSL(57.3 kb), UGT3A1(110 kb) |
| rs10036386 | 12 | 19.2 | 0 | | PDE8B(−21.3 kb), ZBED3(−144.9 kb) |
| rs1991797 | 35 | 90.3 | 0 | | C5orf30(−0.7 kb), PPIP5K2(−130.8 kb) |
| rs114378220 | 1 | 0 | 0 | | CAMK4(−6.3 kb), WDR36(−138.5 kb) |
| rs244674 | 19 | 33.8 | 1 | TCF7 | SKP1(59.9 kb), VDAC1(−78.1 kb) |
| rs2434973 | 46 | 44.3 | 2 | FAM71B, ITK | MED7(−3.9 kb), HAVCR2(−37.7 kb) |
| rs3778607 | 16 | 31.9 | 0 | | IRF4(−12 kb), DUSP22(−111.3 kb) |
| rs544188579 | 259 | 7942.6 | 263 | — | |
| rs2229637 | 109 | 7750.8 | 249 | — | |
| rs200949 | 97 | 2172.7 | 49 | — | |
| rs78306789 | 1 | 0 | 0 | | ZBTB9(−34.8 kb), SYNGAP1(−69.3 kb) |
| rs6457834 | 40 | 1016.2 | 15 | — | |
| rs1724088 | 28 | 84.7 | 0 | | FGD2(−3 kb), MTCH1(−22.4 kb) |
| rs1317840 | 8 | 4 | 0 | | VEGFA(−65.2 kb), MRPS18A(−148.6 kb) |
| rs72928038 | 3 | 125.3 | 0 | | BACH2(0.7 kb), GJA10(−276.2 kb) |
| rs654537 | 9 | 51.8 | 0 | | BACH2(4 kb), MAP3K7(294.3 kb) |
| rs7758816 | 5 | 3.5 | 0 | | FYN(13.3 kb), TRAF3IP2(−170.8 kb) |
| rs2223385 | 42 | 53.8 | 0 | | MYB(25.3 kb), HBS1L(−47.5 kb) |
| rs7753634 | 145 | 411.2 | 1 | AHI1 | PDE7B(27.4 kb), MYB(−231.8 kb) |
| rs187855607 | 18 | 143.2 | 0 | | SASH1(15.1 kb), UST(419.3 kb) |
| rs9497965 | 2 | 7 | 0 | | SASH1(142.7 kb) |
| rs1079418 | 15 | 19.7 | 0 | | PDE10A(−50.9 kb), C6orf118(−317.8 kb) |
| rs9366078 | 15 | 35 | 1 | RNASET2 | RNASET2(0 kb), AL159163.1(−0.6 kb) |
| rs10277273 | 15 | 20.5 | 0 | | AP5Z1(29.7 kb), FOXK1(−43.1 kb) |
| rs202157095 | 42 | 55.2 | 1 | ELMO1 | ELMO1(51.2 kb), GPR141(285.9 kb) |
| rs11765986 | 86 | 396.8 | 3 | RSBN1L, | |
| | | | | TMEM60, | |
| | | | | PHTF2 | |
| rs221781 | 17 | 100.8 | 5 | — | |
| rs35144535 | 47 | 169 | 2 | IRF5,TNPO3 | KCP(−12.6 kb), AC011005.1(34.3 kb) |
| rs2921071 | 18 | 78.2 | 0 | | PRAG1(−60.6 kb), CLDN23(176.7 kb) |
| rs1032129 | 11 | 77.6 | 1 | TNFRSF11B | COLEC10(102.1 kb), MAL2(243.3 kb) |
| rs2445610 | 13 | 18.4 | 0 | | POU5F1B(137.3 kb) |
| rs759649 | 77 | 107.9 | 0 | | MYC(−406.5 kb) |
| rs1561924 | 77 | 72.6 | 0 | | |
| rs1810396 | 55 | 49.5 | 0 | | TG(−38.4 kb), PHF20L1(−130 kb) |
| rs7005834 | 9 | 9.6 | 0 | | CCN4(−6.9 kb), NDRG1(89.7 kb) |
| rs11783023 | 28 | 55.1 | 1 | AGO2 | CHRAC1(−84.3 kb), TRAPPC9(−137 kb) |
| rs911760 | 6 | 16.2 | 1 | PLGRKT | CD274(12.1 kb), PDCD1LG2(72.1 kb) |
| rs2123340 | 19 | 37 | 0 | | IFNE(−69.7 kb), IFNA1(−111.6 kb) |
| rs925489 | 23 | 18.7 | 0 | | FOXE1(61.6 kb), XPA(−75.6 kb) |
| 9:110602043_AC_A | 1 | 0 | 0 | | KLF4(−350 kb) |
| rs1107342 | 80 | 145.6 | 1 | NEK6 | PSMB7(29.5 kb), ADGRD2(66.1 kb) |
| rs9697210 | 37 | 225.5 | 10 | — | |
| rs12722518 | 6 | 6.8 | 0 | | IL2RA(22.4 kb), RBM17(49.1 kb) |
| rs7090530 | 3 | 12.1 | 1 | IL2RA | RBM17(20.1 kb), IL15RA(−79.3 kb) |
| rs947474 | 8 | 17.4 | 0 | | PFKFB3(−145.6 kb), PRKCQ(214.4 kb) |
| rs71508903 | 1 | 0 | 0 | | ARID5B(−118.4 kb), RTKN2(248.7 kb) |
| rs7905731 | 31 | 88.5 | 1 | RTKN2 | ZNF365(78.1 kb), ARID5B(−306.1 kb) |
| rs143627643 | 25 | 19.5 | 0 | | PTEN(−169.9 kb), KLLN(−170 kb) |
| rs10748781 | 30 | 19.4 | 1 | NKX2-3 | GOT1(−83.7 kb), SLC25A28(86.7 kb) |
| rs7936397 | 75 | 69.4 | 4 | — | |
| rs10742340 | 41 | 53.7 | 0 | | SLC1A2(64 kb), CD44(−103.6 kb) |
| rs97384 | 45 | 76.9 | 4 | — | |
| rs479777 | 29 | 123.8 | 11 | — | |
| rs4409785 | 1 | 0 | 0 | | FAM76B(211.3 kb), CEP57(212.2 kb) |
| rs76169968 | 224 | 296.8 | 4 | — | |
| rs10844682 | 51 | 70.1 | 1 | CLECL1 | CD69(3.4 kb), CLEC2D(−17.7 kb) |
| 12:9924451_TA_T | 18 | 10.9 | 0 | | CD69(−3.1 kb), CLECL1(−30.7 kb) |
| rs7971751 | 38 | 112.7 | 4 | — | |
| rs12582330 | 8 | 46.2 | 1 | C12orf42 | STAB2(47.4 kb), NT5DC3(301.4 kb) |
| rs3184504 | 1 | 0 | 0 | | SH2B3(−40.9 kb), PHETA1(−83.1kb) |
| rs9511151 | 34 | 15 | 0 | | SPATA13(−38.3 kb), C1QTNF9(93.1 kb) |
| rs7333647 | 36 | 14.7 | 0 | | SPATA13(−38.4 kb), C1QTNF9(93.3 kb) |
| rs76428106 | 1 | 0 | 0 | | URAD(−41.2 kb), CDX2(−60.6 kb) |
| rs2324919 | 81 | 130.8 | 0 | | TNFSF11(65 kb), AKAP11(−106.2 kb) |
| rs35407628 | 56 | 262 | 3 | UBAC2, | |
| | | | | GPR18, | |
| | | | | GPR183 | |
| rs2208397 | 13 | 31.7 | 0 | | RAD51B(−441.9 kb), ZFYVE26(−445.1 kb) |
| rs76247422 | 50 | 85.7 | 0 | | C14orf177(485 kb) |
| rs34678053 | 11 | 57.2 | 1 | IGHG2 | IGHA1(7.7 kb), IGHG4(−17.7 kb) |
| rs7173565 | 14 | 29.9 | 1 | RASGRP1 | FAM98B(−81.8 kb), SPRED1(−283.1 kb) |
| rs34916596 | 1 | 0 | 0 | | CREBBP(−81.3 kb), ADCY9(154.4 kb) |
| rs182787262 | 5 | 425.1 | 4 | — | |
| rs4494538 | 43 | 59.4 | 0 | | BRD7(0.1 kb), ADCY7(−21.4 kb) |
| rs8054578 | 28 | 321.2 | 0 | | MAF(5.8 kb) |
| rs61759532 | 3 | 13.4 | 2 | NEURL4, | GPS2(−8.3 kb), KCTD11(14.8 kb) |
| | | | | ACAP1 | |
| 17:8843722_CA_C | 24 | 43.9 | 1 | PIK3R5 | PIK3R5(−16.9 kb), NTN1(48.2 kb) |
| rs12325861 | 5 | 10.9 | 0 | | HSPB9(−3.7 kb), KAT2A(−5.1 kb) |
| rs4794063 | 16 | 496.9 | 6 | — | |
| rs2847297 | 12 | 29.5 | 0 | | PSMG2(−71.9 kb), CEP76(−72.2 kb) |
| rs693939 | 21 | 322.2 | 2 | SS18, PSMA8 | TAF4B(5.2 kb), KCTD1(327.8 kb) |
| rs1790963 | 37 | 32 | 0 | | CD226(71.4 kb), RTTN(329.2 kb) |
| rs1562722 | 13 | 62.3 | 0 | | NFATC1(−18.1 kb), CTDP1(203.5 kb) |
| rs10424978 | 2 | 0.1 | 0 | | TICAM1(−5.8 kb), PLIN3(30.1 kb) |
| rs7248104 | 23 | 29.5 | 0 | | INSR(53.6 kb), AC119396.1(114.9 kb) |
| rs11085727 | 8 | 32.3 | 1 | TYK2 | ICAM3(−9.7 kb), RAVER1(−15.7 kb) |
| rs12973608 | 25 | 25 | 2 | IQCN, JUND | AC008397.2(−17.3 kb), |
| | | | | | AC008397.1(−17.3 kb) |
| rs12980063 | 2 | 0.4 | 0 | | CPT1C(−2.6 kb), ADM5(−5.1 kb) |
| rs2745803 | 1 | 0 | 0 | | SNX5(89.7 kb), MGME1(90 kb) |
| rs2403967 | 50 | 19.7 | 0 | | USP25(299.8 kb), NRIP1(-345.5 kb) |
| rs714027 | 12 | 90.9 | 0 | | HORMAD2(−9.8 kb), LIF(65 kb) |
| rs229540 | 8 | 10.6 | 1 | C1QTNF6 | SSTR3(14.9 kb), IL2RB(−35.5 kb) |
| rs4820437 | 247 | 496.2 | 12 | — | |

TABLE 8
Candidate genes within and near credible sets

| signal.lead | n.variants | span.kb | n.within | gene.within | gene.dist |
|---|---|---|---|---|---|
| rs159960 | 60 | 226.5 | 0 | | SLC45A1(−53.7 kb), RERE(219.4 kb) |
| rs2476601 | 2 | 73.8 | 1 | RSBN1 | PHTF1(−1.7 kb), PTPN22(36.8 kb) |
| rs78037977 | 11 | 41.2 | 0 | | FASLG(−46.3 kb), SUCO(−172.3 kb) |
| rs16843742 | 15 | 73.6 | 1 | PTPRC | ATP6V1G3(−88.6 kb), NEK7(−472.5 kb) |
| rs9309267 | 30 | 105.8 | 1 | PPP4R3B | PNPT1(29.8 kb), CFAP36(−38.5 kb) |
| rs57874285 | 33 | 150.8 | 1 | BCL2L11 | ACOXL(−371.7 kb), BUB1(−426.2 kb) |
| rs2111485 | 2 | 13.5 | 0 | | FAP(−10.6 kb), IFIH1(51.1 kb) |
| rs3096860 | 26 | 99.9 | 1 | CTLA4 | ICOS(7 kb), CD28(−123.2 kb) |
| rs60135207 | 61 | 102.2 | 1 | FOXP1 | FOXP1(9.3 kb), EIF4E3(191.2 kb) |
| rs140226894 | 64 | 18.2 | 1 | CD80 | CD80(−0.1 kb), ADPRH(1.4 kb) |
| rs2037184 | 16 | 353.9 | 1 | LPP | BCL6(−265 kb), AC072022.2(−266.8 kb) |
| rs13076312 | 11 | 43.9 | 0 | | LPP(−146.1 kb) |
| rs13136820 | 4 | 7.1 | 0 | | CHRNA9(29 kb), RHOH(−102.2 kb) |
| rs6904596 | 155 | 2045.9 | 72 | — | |
| rs2967 | 36 | 7450.1 | 230 | — | |
| rs72928038 | 57 | 198.7 | 1 | BACH2 | GJA10(−210 kb), CASP8AP2(−274.6 kb) |
| rs2247314 | 62 | 40.4 | 1 | RNASET2 | AL159163.1(−0.4 kb), FGFR1OP(2.5 kb) |
| rs117744081 | 1 | 0 | 0 | | AC004593.3(29.7 kb), CPVL(53.8 kb) |
| rs10087240 | 29 | 389.4 | 1 | MYC | POU5F1B(−304.7 kb) |
| rs853308 | 40 | 56 | 0 | | TG(−42.6 kb), PHF20L1(−134.2 kb) |
| rs11781004 | 9 | 9.6 | 0 | | CCN4(−6.9 kb), NDRG1(89.7 kb) |
| rs7079460 | 37 | 122.4 | 2 | IL2RA, RBM17 | IL15RA(−38.1 kb), PFKFB3(64.8 kb) |
| rs706779 | 1 | 0 | 0 | | IL2RA(5.5 kb), RBM17(32.2 kb) |
| rs3814231 | 14 | 51.2 | 0 | | CASP7(−1.4 kb), NRAP(−17.2 kb) |
| rs1043101 | 40 | 58.5 | 0 | | SLC1A2(59.2 kb), CD44(−103.6 kb) |
| rs12295166 | 3 | 64.5 | 0 | | TYR(−0.7 kb), GRM5(−114.9 kb) |
| rs1126809 | 1 | 0 | 0 | | TYR(−106.9 kb), NOX4(206.6 kb) |
| rs11021232 | 2 | 9.4 | 0 | | FAM76B(201.9 kb), CEP57(202.9 kb) |
| rs644515 | 16 | 22.6 | 0 | | FLI1(−46.4 kb), KCNJ1(79.5 kb) |
| rs772921 | 12 | 85.8 | 3 | SUOX, IKZF4, | |
| | | | | RPS26 | |
| rs7137828 | 7 | 345.7 | 4 | — | |
| rs111260018 | 39 | 174.8 | 0 | | |
| rs8192917 | 16 | 21 | 1 | GZMB | GZMH(−21.4 kb), CTSG(−54.8 kb) |
| rs1635168 | 51 | 131.5 | 0 | | HERC2(23.4 kb), OCA2(−67.9 kb) |
| rs8083511 | 2 | 1.9 | 0 | | TNFRSF11A(−36.1 kb), ZCCHC2(159.7 kb) |
| rs4807000 | 5 | 6.9 | 1 | TICAM1 | PLIN3(30.1 kb), FEM1A(−38.9 kb) |
| rs2082481 | 8 | 17.8 | 2 | BCL2L12, IRF3 | IRF3(0.1 kb), SCAF1(−5.8 kb) |
| rs6059655 | 3 | 424.7 | 4 | — | |
| rs9981980 | 1 | 0 | 0 | | UBASH3A(−2.1 kb), TMPRSS3(−9.9 kb) |
| rs111462340 | 4 | 10.3 | 0 | | UBASH3A(−14.3 kb), TMPRSS3(−22.1 kb) |
| rs229527 | 8 | 9.8 | 1 | C1QTNF6 | SSTR3(15.7 kb), IL2RB(−35.5 kb) |
| rs9611562 | 4 | 40.2 | 0 | | TEF(9.1 kb), ZC3H7B(−31.1 kb) |

### Example 5. Genetic Variation Affects Risk of Thyroid irAE During Anti-PD-L1 Treatment

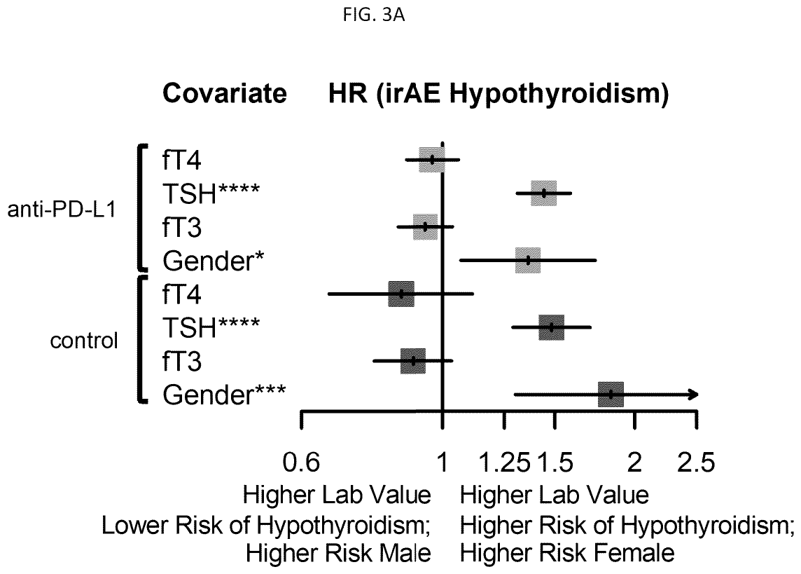

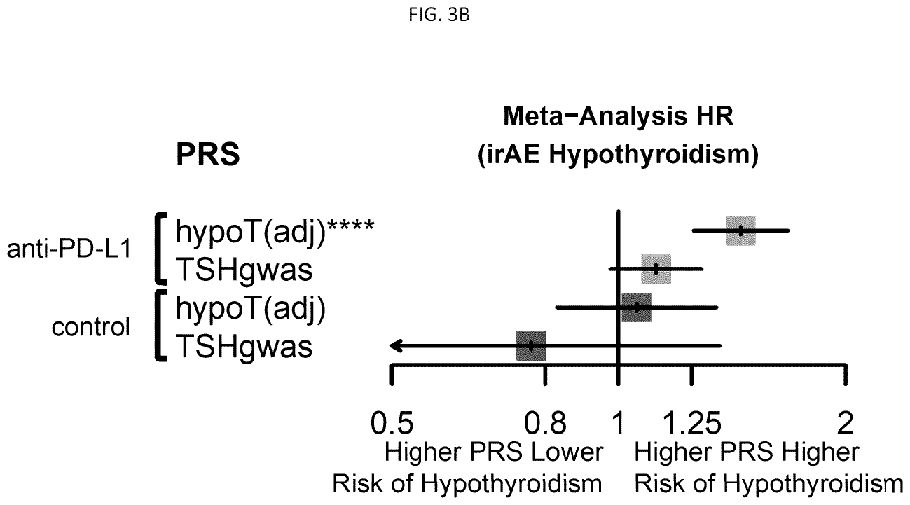

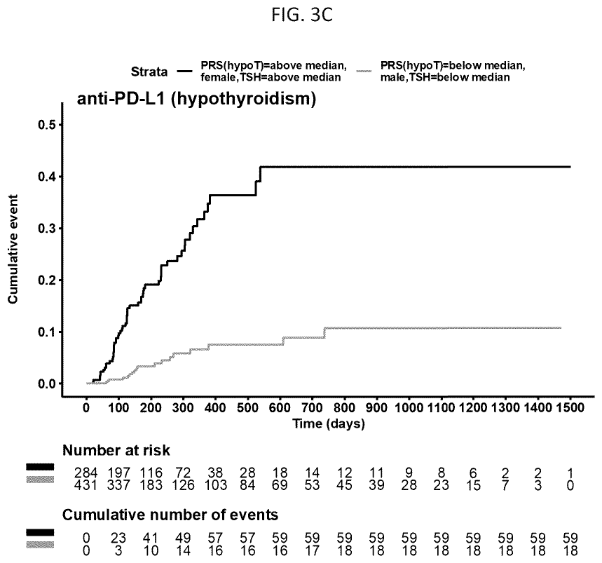

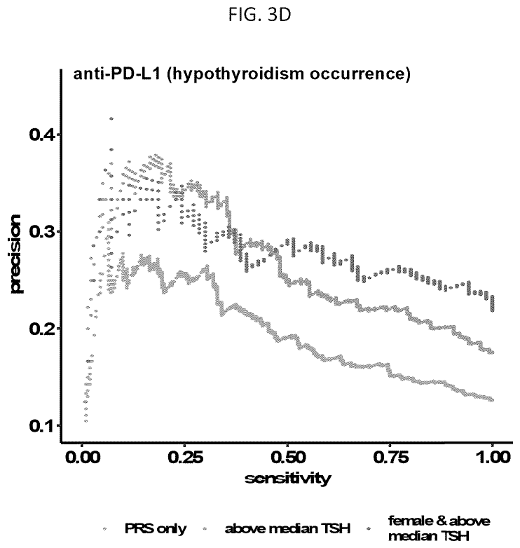

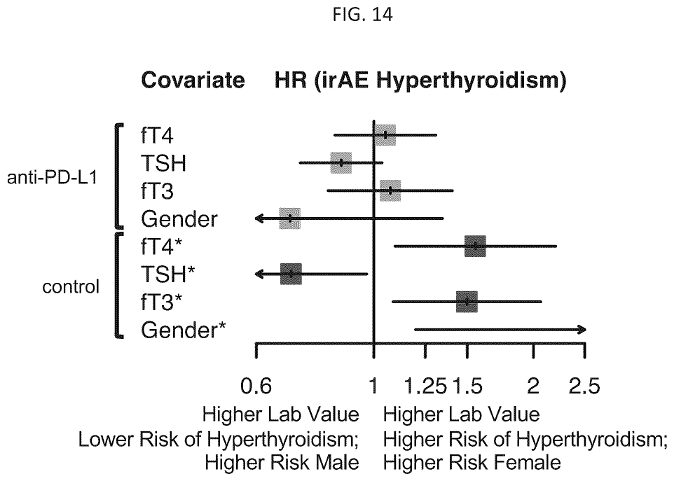

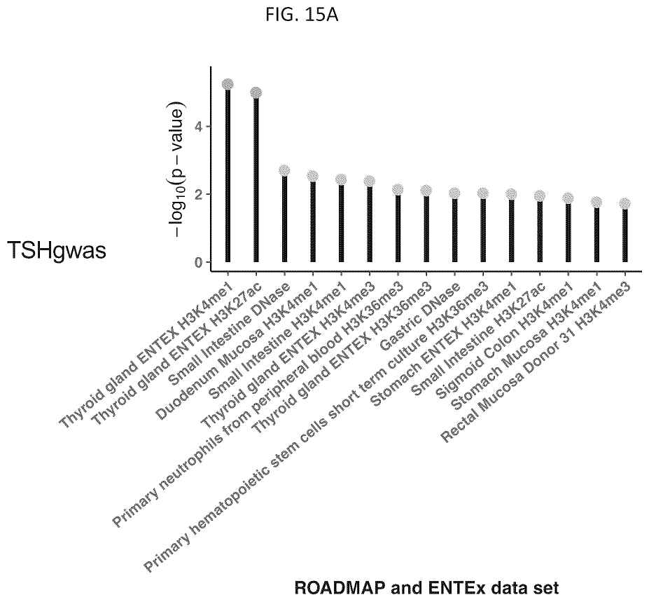

Genetic and environmental factors together may contribute to damage to the thyroid of patients over the course of their lifetime prior to cancer treatment. Hypothyroidism in human populations is more common in females than males, and patients may have differing levels of thyroid function (Chaker et al., Lancet, 390(10101): 1550-1562, 2017). We additionally considered whether these factors were relevant to risk of hypothyroidism irAEs in cancer patients treated with atezolizumab. Hypothyroidism is diagnosed by both patient symptoms and lab measurements. Within the cohort assessed herein, thyroid stimulating hormone (TSH), free thyroxine (fT4), and free triiodothyronine (MT) were measured in patients prior to treatment. The relevance of these lab measures and gender to risk of hypothyroidism irAEs during cancer treatment was assessed. Using a multivariable Cox model, it was determined that measured pre-treatment TSH levels and gender, but not pre-treatment fT4 or fT3 levels, were independently associated with increased risk of hypothyroidism irAEs, but not hyperthyroidism, in both the atezolizumab and the control arms (FIGS. 3A and 14).

i. Association Between Thyroid irAEs and Hormone Levels and Gender

An individual participant data meta-analysis approach was used to determine whether there exists an association between the risk of thyroid irAEs and the following factors: thyroid hormone levels TSH, fT4, and fT3 at baseline and gender. TSH, fT4, and fT3 levels were normalized to the quantiles of a standard normal (using qqnorm function in R). The normalization allowed comparison across hormone levels. The gender variable was encoded as female=1 and male=0. To conduct the meta-analysis, the coxme package in R was used. The model allowed for a random effect on each of the hormone levels and gender, allowing for differing effect sizes across each trial arm. The model also allowed for a differing baseline hazard across each trial arm. The 95% confidence intervals around the hazard ratios expressed in unit normalized hormone levels were reported.

The multivariable Cox model showed that baseline TSH levels and gender, but not baseline fT4 or fT3 levels, were independently associated with increased risk of hypothyroidism in both the atezolizumab and the control arms (FIG. 3A). No association was observed between baseline hormone levels for atezolizumab combinations and risk for hyperthyroidism irAEs (FIG. 14).

ii. Cross-Validation Estimation of PRS PPV, Sensitivity, and Effect Size in the UK Biobank

To estimate the PPV and sensitivity (precision and recall) curve for a hypothyroidism PRS as applied to population occurrence of hypothyroidism, 4-fold cross validation was performed in the UK Biobank. Specifically, a hypothyroidism GWAS was conducted on a training fold using both ICD code diagnosed and self-reported cases using SAIGE (Zhou et al., Nat. Genet., 50: 1335-1341, 2018). Then, a PRS was created using summary statistics from this training fold using GCTA-COJO forward selection and by selecting the max PPA variant to include in the PRS—the algorithm was identical to that used to create the PRS used in the study of cancer patients from the atezolizumab trials described herein. Then, the PRS was applied to the test fold from the UK Biobank and quantile normalized the PRS values to the quantiles of a standard normal in the test fold so that they were comparable across folds. The test folds were combined and estimated the positive predictive value (PPV) and sensitivity (precision and recall) curve were estimated for varying thresholds on the PRS where above threshold designated predicted case status. Effect size between cases and controls were estimated by computing the mean PRS value for cases and the mean PRS value for controls within each test fold. The effect size was estimated as the absolute value of the difference between the means divided by the standard deviation of the PRS values computed in the test folds. The cross-validated effect size estimate was the average of the within test fold estimated effect sizes.

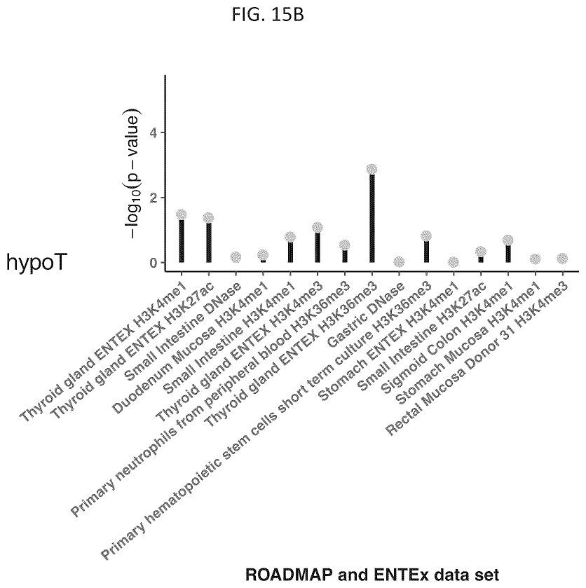

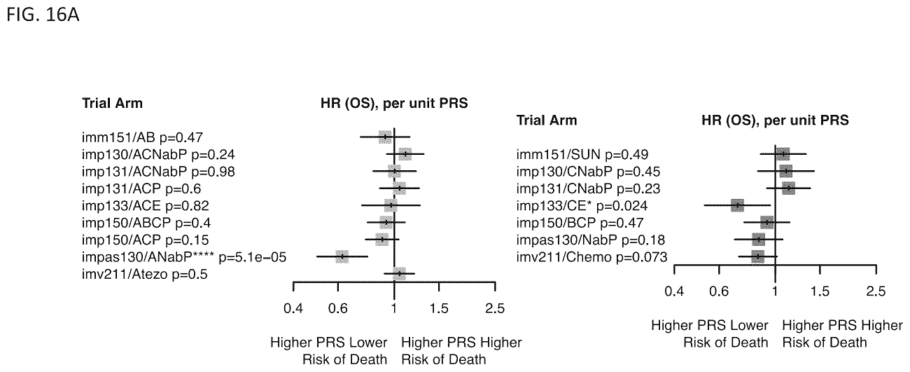

Given that measured pre-treatment TSH and gender were independently associated with risk of hypothyroidism irAEs during atezolizumab treatment, this raised the possibility that the PRS and hypothyroidism irAE risk association might be explained by these factors. Genetic variation affecting thyroid function to set baseline TSH levels might contribute to risk of hypothyroidism irAEs in atezolizumab treated patients. To investigate whether risk for hypothyroidism irAEs might be due to genetic factors affecting TSH levels, a PRS was constructed using a GWAS of TSH levels from European individuals not receiving any medication for thyroid dysfunction (Teumer et al., Nat Commun, 9(1): 4455, 2018). In contrast to the hypothyroidism GWAS, enrichment in thyroid-specific enhancers was clearly present in this GWAS, with no evidence of immune cell enrichment (FIGS. 11, 15A, and 15B). While the PRS was correlated with measured pre-treatment TSH levels (Spearman's rs=0.228) in cancer patients, no association of the TSH PRS with increased risk of atezolizumab-induced hypothyroidism was observed (FIG. 3B).

A weaker correlation was found between the hypothyroidism PRS and measured pre-treatment TSH levels (Spearman's rs=0.109), suggesting that damage to the thyroid due to autoimmunity over a patient's lifetime prior to cancer treatment may account for the findings. To address this possibility, the analysis of the association between the hypothyroidism PRS and hypothyroidism irAEs was repeated including measured pre-treatment TSH levels and gender as covariates in the analysis. The association in atezolizumab-treated patients was unchanged (HR=1.45, 95% CI 1.25-1.67 per unit normalized PRS; FIG. 3B). Together, these additional analyses indicate that the hypothyroidism PRS and irAE risk association is not explained by thyroid function prior to atezolizumab treatment or by gender, but arises from the effect of genetic variation during the course of atezolizumab treatment.

As several independent risk factors associated with hypothyroidism irAEs were identified in atezolizumab-treated patients, it was explored whether these factors could be combined to identify sub-groups at high or low risk and to develop a pre-treatment predictor. Combining each independent risk factor, risk of hypothyroidism irAEs was found to be 6.85 times higher (95% CI HR=3.49-13.44) in female anti-PD-L1 treated patients with above-median hypothyroidism PRS and above-median baseline TSH levels as compared to male anti-PD-L1 treated patients with below-median PRS and below-median baseline TSH levels (FIG. 3C). To evaluate these factors as pre-treatment predictors of hypothyroidism irAE, the positive predictive value (PPV) and sensitivity (precision and recall) with which the hypothyroidism PRS correctly predicted hypothyroidism irAE occurrence at varying thresholds of the PRS in atezolizumab-treated patients were quantified. These metrics were used due to the large imbalance between the positive and negative cases in these data. These properties were considered in patient subgroups with higher incidence of hypothyroidism irAEs, as delineated by an above-median baseline TSH, and additionally by gender (FIG. 3D). These curves were compared to a 4-fold cross validation estimate of the PPV and sensitivity for predicting population hypothyroidism cases by PRS in the UK Biobank. Although the PRS had a PPV in atezolizumab-treated patients that was higher than its population PPV, it did not achieve near perfect prediction even at a high cutoff with low sensitivity.

### Example 6. Hypothyroidism PRS is Associated with OS in Anti-PD-L1-treated TNBC Patients

The low precision of the hypothyroidism PRS as a predictor for hypothyroidism irAEs further limits its precision and sensitivity as predictor for OS. Both high precision and sensitivity is needed to recapitulate the association between the event occurrence and OS we previously observed (FIG. 1B). Given that data were studied across cancers, an exception to this observation might reflect the importance of genetic variation associated with lifetime risk for thyroid autoimmunity within the cancer indication. It was next assessed whether the hypothyroidism PRS was associated directly with outcome measures within each of the analyzed trial arms.

The association between the hypothyroidism PRS and OS was tested using a Cox proportional hazards model within each trial arm in the data set. The p-value corresponded to the coefficient associated with the PRS, which was normalized to the quantiles of a standard normal. All hazard ratios were expressed in normalized unit PRS. The model included genotype eigenvectors, presence or absence of liver metastases, and an indicator variable for baseline ECOG>0 status versus baseline ECOG=0 status as covariates. p-values for the PRS term were computed for each trial arm and compared to the p-value for Bonferroni significance to account for multiple testing across each trial arm.

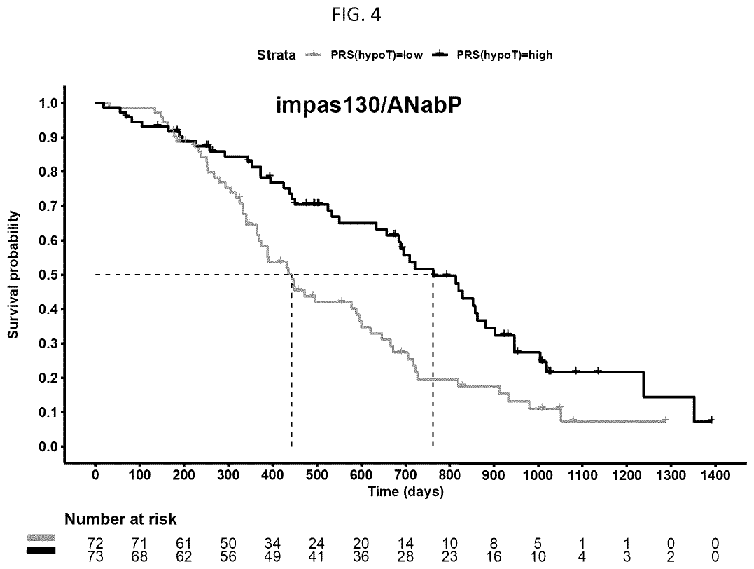

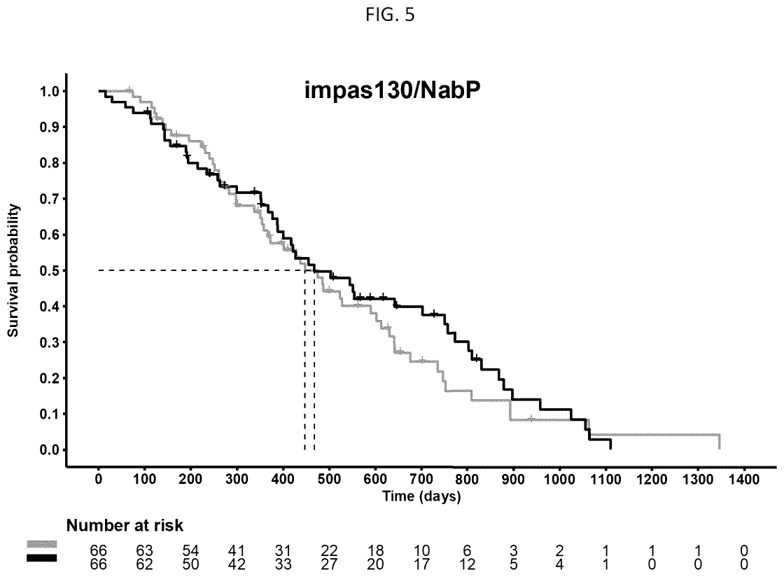

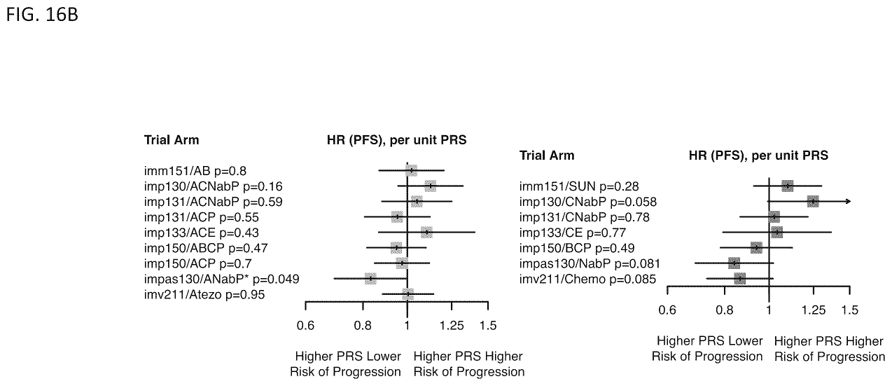

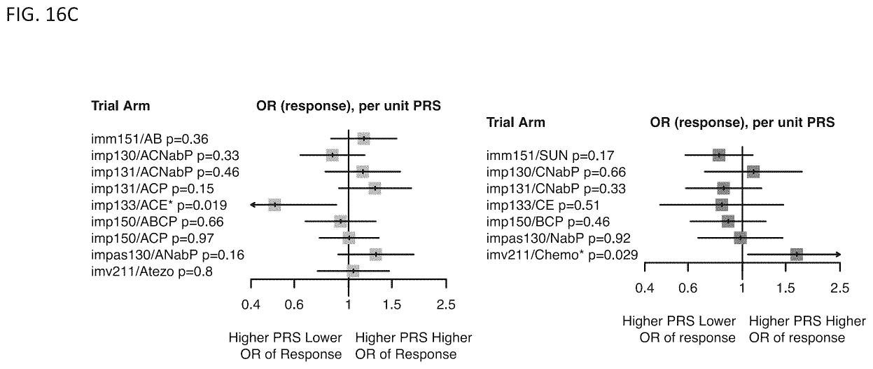

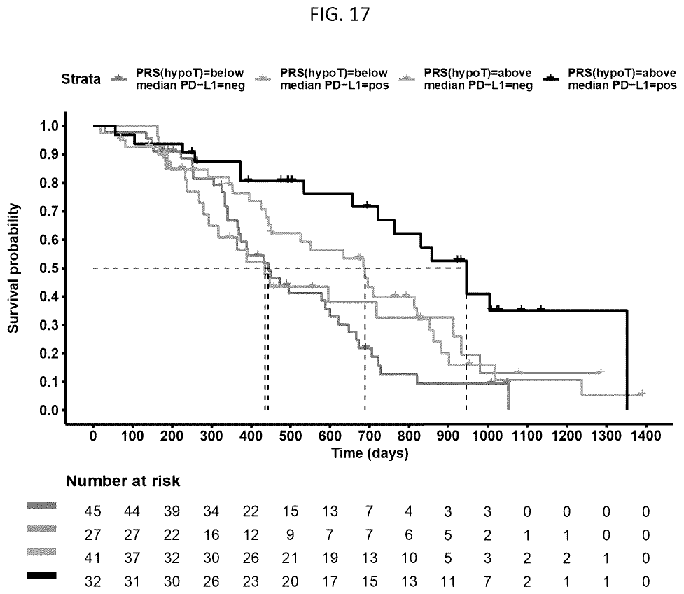

Consistent with its limited precision and sensitivity, the hypothyroidism PRS was not associated with OS, PFS, or tumor response across most trial arms (FIGS. 16A-16C). Analyses was limited to European (EUR) ancestry individuals in each trial arm. Sample sizes per trial arm for EUR ancestry patients are provided in Table 4. However, a positive association between the hypothyroidism PRS and lower risk of death was identified in triple negative breast cancer (TNBC) patients in the atezolizumab plus nab-paclitaxel arm of IMpassion130. The corresponding p-value met the cutoff for Bonferroni significance, accounting for the number of outcome measures and trial arms tested (p=5.06×10⁻⁵, HR=0.62, 95% CI 0.49-0.78 per unit normalized PRS). No association (p=0.17, HR=0.86, 95% CI 0.69-1.07 per unit normalized PRS) was observed in the placebo plus nab-paclitaxel arm of this trial. (FIGS. 4 and 5). The association observed in the atezolizumab arm was robust to inclusion of PD-L1 positivity in the tumor as a covariate, indicating the PRS was independently associated with survival in this trial (adjusted HR=0.65, 95% CI HR=0.37-0.7551-0.83 per unit normalized PRS) (FIG. 17).

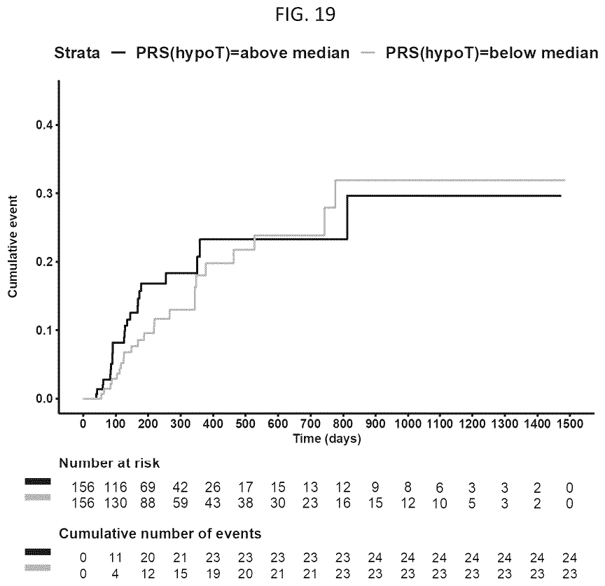

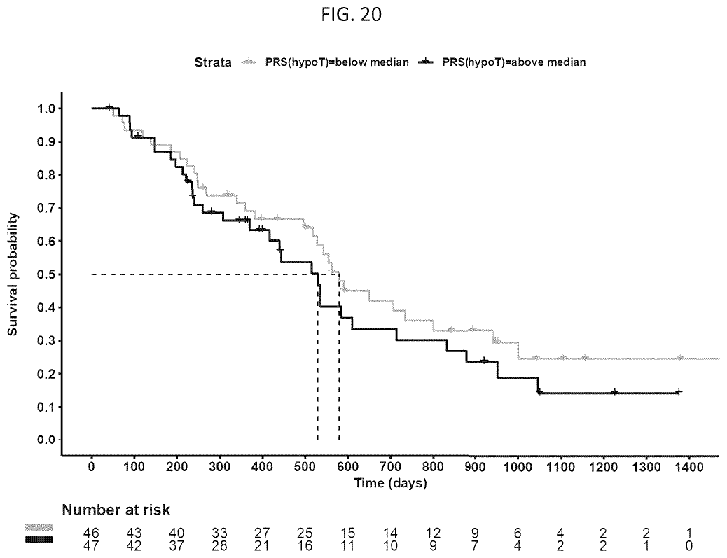

TNBC varies in prevalence across ethnicities, with higher prevalence in African Americans (Dietze et al., Nat. Rev. Cancer, 15: 248-254, 2015). However, the hypothyroidism PRS was constructed using data from Europeans. Therefore, it was assessed whether the PRS associations we observed in atezolizumab treated patients were transferable across ethnicities. Hypothyroidism irAE data from 312 atezolizumab-treated patients from the trials studied herein that did not meet the cutoff for European ancestry and were excluded from the initial analysis were analyzed (FIG. 18; Table 4). In this set of patients, no evidence was found that the UK Biobank-derived hypothyroidism PRS was associated with risk of hypothyroidism irAEs (meta-analysis, p=0.09; HR=1.43, 95% CI 0.95-2.18 per unit normalized PRS; FIG. 19). Within this set, no evidence of an association was found between the hypothyroidism PRS and OS in 93 atezolizumab-treated TNBC patients from the IMpassion130 trial (p=0.53, HR=1.1, 95% CI 0.81-1.49) (FIG. 20). Consistent with prior studies that have shown that PRS have limited trans-ethnic transferability, our findings, at present, are limited to cancer patients of European ancestry (Martin et al., a. J. Hum. Genet., 100: 635-649, 2017).

### I. CONCLUSIONS

The results provided herein indicate that development of hypothyroidism is associated with longer OS in cancer patients. This observation is not a characteristic of PD-1 checkpoint inhibition alone; a similar association occurred in the sunitinib arm of IMmotion151, a trial in which a consistent adverse event reporting methodology was applied. Although the analysis accounted for survival bias, the direction of causality is difficult to discern. Induction of hypothyroidism may lead to physiological changes favorable for cancer survival. However, responding patients might also be more vulnerable to development of hypothyroidism. The analysis establishes that, in contrast to sunitinib-induced hypothyroidism, atezolizumab-induced hypothyroidism has a genetic basis and is driven by variants that contribute to lifetime risk of autoimmunity and, to a lesser extent, tissue-specific susceptibility.

Although the occurrence of hypothyroidism irAEs was correlated with longer OS, the hypothyroidism PRS was not associated with longer OS in the majority of studies analyzed. Recapitulating the link between hypothyroidism and OS will require additional, independent pre-treatment predictors of hypothyroidism irAEs. The one exception to this observation was in the atezolizumab treatment arm of IMpassion130. Women with thyroid cancer are at increased risk for subsequent breast cancer, and there is an increased risk of breast cancer after thyroid cancer (Bolf et al., Cancer Epidemiol Biomarkers Prev, 28(4): 643-649, 2019). Epidemiological studies have linked hypothyroidism and thyroid autoantibodies to risk and positive prognosis in breast cancer, leading to the hypothesis that shared antigens might exist between the thyroid and breast tissues (Muller et al., Semin Cancer Biol, 2019).

PD-1 checkpoint blockade substantially lowers the threshold for thyroid autoimmunity. Whether an individual patient will develop atezolizumab-induced hypothyroidism depends, in part, on variants in their genome that contribute to the balance between tolerance and autoimmunity. Genes in proximity to or functionally linked to these variants may identify therapeutically relevant modifiers of the systemic immune response to PD-1 checkpoint blockade. For instance, a region near CTLA4 that contributed substantially to the association between the hypothyroidism PRS and atezolizumab-induced irAEs was identified. Combination therapies that block both CTLA4 and PD-1 have been shown to achieve elevated anti-tumor responses in the clinic (Postow et al., N Engl J Med, 372(21): 2006-2017, 2015). Contributing variants in and near CBLB, PTPN22, and CD69 were also identified. Deletion of any one of these genes in preclinical models has been shown to overcome resistance to TGFβ-mediated immunosuppression in the tumor microenvironment (Brownlie et al., Nat Commun, 8(1): 1343, 2017; Wohlfert et al., J Immunol, 176(3): 1316-1320, 2006; Esplugues et al., J Exp Med, 197(9): 1093-1106, 2003).

The concept of a cancer-immune set point considers not only the immune profile of a tumor, but also the state of patient's immune system to explain why some immunotherapy-treated patients mount effective and safe anti-tumor responses and others fail to respond or develop immune toxicities. The present study focused on atezolizumab-induced hypothyroidism irAEs, which are noteworthy given that this study shows that they are common and associated with longer patient survival across cancers and chemotherapy combinations. PRS derived from a GWAS of hypothyroidism or vitiligo cases and controls in European populations quantify one dimension of a patient's cancer-immune set point: lifetime genetic susceptibility for tissue-specific autoimmunity. This lifetime genetic susceptibility is correlated with risk of hypothyroidism irAEs during the shorter duration of atezolizumab treatment. This implies that genetic mechanisms that contribute to thyroid autoimmunity and vitiligo over a lifetime are magnified and accelerated to increase risk of hypothyroidism irAEs during cancer treatment with atezolizumab.

Derived from loci in the genome, PRS provide insight into the mechanisms of hypothyroidism irAEs. Using cross-validation and survival lasso, it was determined that a subset of variants in the hypothyroidism and vitiligo PRS were retained by the regression model to explain variation in hypothyroidism irAE risk in atezolizumab-treated patients. One of the most intriguing loci selected by lasso from both the hypothyroidism and vitiligo PRS was in the intron of the LPP gene. Both eQTL evidence and promoter capture Hi-C data indicate that that this locus affects BCL6, the lineage-defining transcription factor for Tfh cells, whose TSS was 692 kb away from the credible set. Given that PD-1 is a surface marker of Tfh cells, it is hypothesized that atezolizumab disrupts critical interactions required for B-cell maturation and antibody affinity to contribute to irAE risk (Crotty et al., Immunity, 50: 1132-1148, 2019). The variants retained also included the extensively studied R620W (rs2476601) variant in PTPN22, highlighting the role of T-cell and B-cell receptor signaling (Vang et al., Sci. Signal., 11, 2018). Although this approach has important limitations, as demonstrated by the variant in the intron of LPP, examining the genes near credible sets to which a retained variant belongs also highlighted genes involved in T-cell priming (CTLA4) and activation (CD69). Given that these retained variants contribute to inter-individual variation in a phenotype arising due to atezolizumab treatment, the present study introduces an approach to identify pathways and genes modulated during PD-1 blockade.

Genetic susceptibility to thyroid autoimmunity may translate into survival benefit in atezolizumab plus nab-paclitaxel treated TNBC patients. Evidence of immune cross-reactivity has been found in PD-1 checkpoint inhibitor myocarditis, wherein T-cell clones in the myocardium at autopsy were the same T-cell clones as in the patient's tumor (Johnson et al., N. Engl. J. Med., 375: 1749-1755, 2016). Thyroid auto-antibodies have been associated, in some contexts, with better breast cancer prognosis (Muller and Barrett-Lee, Semin. Cancer Biol., 64: 122-1, 2020). Immune cells cross-reactive to antigens in both breast and thyroid, both glandular tissues, could explain the association between the PRS and OS in atezolizumab plus nab-paclitaxel treated TNBC patients. Even in the absence of cross-reactivity, immune responses mediated by Tfh cells and B-cells might play a significant role in extending TNBC patient survival. Investigation of these hypotheses could provide avenues for therapeutic strategies and approaches to stratify patients in TNBC.

The present findings have implications for clinical management of cancer patients treated with PD-1 checkpoint inhibitors and for the use of PRS more broadly. When combined with additional risk factors, the hypothyroidism PRS identifies subgroups with a >6-fold difference in risk for atezolizumab-induced hypothyroidism irAEs. As most hypothyroidism irAEs occur within the first year of treatment with atezolizumab, this application of a PRS has greater potential for meaningful impact on individual patients than use of PRS to report lifetime risk. Yet, the use of PRS in clinical settings has an important limitation. At present the trans-ethnic transferability of PRS is limited (Martin et al., Am. J. Hum. Genet., 100: 635-649, 2017). Non-European populations are underrepresented in GWAS and correspondingly in PRS (Mills and Rahal, Nat. Genet., 52: 242-243, 2020). Algorithms that enhance the trans-ethnic transferability of PRS remain an active area of research. These important challenges must be overcome to ensure all patients benefit from the information provided by PRS.

In summary, hypothyroidism irAEs arise as a consequence of the systemic immune response to PD-1 blockade. This present study demonstrates that genetic variation associated with lifetime risk of thyroid autoimmunity shapes this systemic immune response to contribute to irAE susceptibility during atezolizumab treatment. These findings provide a basis for informing therapeutic strategies that can mitigate risk of immune toxicity and improve the efficacy of PD-1 checkpoint inhibitors.

1. (canceled)

2. A method of treating an individual having a cancer, the method comprising:
(a) determining a polygenic risk score (PRS) for one or both of hypothyroidism and vitiligo from a sample from the individual, wherein the PRS for hypothyroidism is above a hypothyroidism reference PRS and/or the PRS for vitiligo is above a vitiligo reference PRS;
(b) administering an effective amount of atezolizumab to the individual; and
(c) monitoring the individual for symptoms of thyroid dysfunction,
wherein the hypothyroidism reference PRS is a median PRS for hypothyroidism in a reference population of individuals having the cancer and the vitiligo reference PRS is a median PRS for vitiligo in a reference population of individuals having the cancer.

3. The method of claim 2, wherein the cancer is metastatic urothelial carcinoma, non-squamous non-small cell lung cancer (NSCLC), squamous NSCLC, small cell lung cancer (SCLC), renal cell carcinoma (RCC), or triple negative breast cancer (TNBC).

4-9. (canceled)

10. A method of treating an individual having a triple-negative breast cancer (TNBC), the method comprising administering atezolizumab to the individual who has been determined to have a PRS for hypothyroidism that is above a hypothyroidism reference PRS in a sample from the individual, wherein the hypothyroidism reference PRS is a median PRS for hypothyroidism in a reference population of individuals having the cancer.

11-13. (canceled)

14. The method of claim 2, wherein (a) the PRS for vitiligo of the sample from the individual or (b) the PRS for vitiligo of a sample from an individual in the reference population is calculated using the equation:
$$\hat{S} = {\sum }_{i = 1}^{M} {β}_{i} \cdot {G}_{i}$$
wherein:
(i) Ŝ is the PRS for vitiligo;
(ii) M is the number of risk alleles selected from independent genetic signals in a genome-wide association study (GWAS) for vitiligo;
(iii) i represents the index of a given SNP;
(iv) βᵢ is the log odds ratio or conditionally independent odds ratio of the ith SNP; and
(v) Gᵢ={0,1,2} is the number of copies of the SNP in the sample from the individual.

15. The method of claim 14, wherein the risk alleles are selected from Table 7 and/or Table 8.

16. The method of claim 14, wherein the risk alleles are identified in the sample by whole-genome sequencing.

17-19. (canceled)

20. The method of claim 10, wherein (a) the PRS for hypothyroidism of the sample from the individual or (b) the PRS for hypothyroidism of a sample from an individual in the reference population is calculated using the equation:
$$\hat{S} = {\sum }_{i = 1}^{M} {β}_{i} \cdot {G}_{i}$$
wherein:
(i) Ŝ is the PRS for hypothyroidism;
(ii) M is the number of risk alleles selected from independent genetic signals in a genome-wide association study (GWAS) for hypothyroidism;
(iii) i represents the index of a given SNP;
(iv) βᵢ is the log odds ratio or conditionally independent odds ratio of the ith SNP; and
(v) Gᵢ={0,1,2} is the number of copies of the SNP in the sample from the individual.

21. The method of claim 20, wherein the risk alleles are selected from Table 7 or Table 8.

22. The method of claim 20, wherein the risk alleles are identified in the sample by whole-genome sequencing.

23. The method of claim 10, further comprising assessing one or more properties that are positively associated with the predictive capacity of a PRS for hypothyroidism from a sample from the individual before administration of a treatment comprising atezolizumab.

24. The method of claim 23, wherein the property is a level of thyroid-stimulating hormone (TSH) that is above a TSH reference level.

25. The method of claim 24, wherein the TSH reference level is a pre-assigned TSH level.

26. The method of claim 25, wherein the TSH reference level is a median TSH level in the reference population.

27. The method of claim 10, wherein the sample is a whole blood sample.

28. The method of claim 27, wherein the sample is an archival sample, a fresh sample, or a frozen sample.

29-32. (canceled)

33. The method of claim 10, further comprising administering to the individual one or more additional therapeutic agents.

34. The method of claim 10, wherein the treatment comprising atezolizumab is a monotherapy.

35. The method of claim 10, wherein the individual has not been previously treated for the cancer.

36. The method of claim 10, wherein the individual has not been previously administered an immune checkpoint inhibitor.

37. The method of claim 10, wherein the individual is a human of European ancestry.

38. The method of claim 10, wherein the individual is female.

39-41. (canceled)

42. A kit for identifying an individual having a cancer who has an increased likelihood of experiencing treatment-induced thyroid dysfunction during treatment comprising atezolizumab, the kit comprising:
(a) polypeptides or polynucleotides for determining the presence of a set of risk alleles selected from independent genetic signals in a GWAS for hypothyroidism; and/or
(b) polypeptides or polynucleotides capable of determining the presence of a set of risk alleles selected from independent genetic signals in a GWAS for vitiligo; and
(c) instructions for use of the polypeptides or polynucleotides to determine a polygenic risk score (PRS) for one or both of hypothyroidism and vitiligo from a sample from the individual, wherein (i) a PRS for hypothyroidism that is above a hypothyroidism reference PRS or (ii) a PRS for vitiligo that is above a vitiligo reference PRS identifies the individual as one who may have an increased likelihood of experiencing treatment-induced thyroid dysfunction during treatment comprising atezolizumab.

43. A kit for identifying an individual having a TNBC who may benefit from a treatment comprising atezolizumab, the kit comprising:
(a) polypeptides or polynucleotides for determining the presence of a set of risk alleles selected from independent genetic signals in a GWAS for hypothyroidism; and
(b) instructions for use of the polypeptides or polynucleotides to determine a polygenic risk score (PRS) for hypothyroidism from a sample from the individual, wherein a PRS for hypothyroidism that is above a hypothyroidism reference PRS identifies the individual as one who may benefit from a treatment comprising atezolizumab.

44. The kit of claim 43, wherein the risk alleles are selected from Table 7 or Table 8.

45. (canceled)


SEQUENCE LISTING (56 sequences)

SEQ ID NO: 1 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 118 aa, synthetic construct [sequence omitted]

SEQ ID NO: 4 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 5 - protein, 10 aa, synthetic construct
  VARIANT 6: note = MISC_FEATURE - Xaa is Asp or Gly
GFTFSXSWIH

SEQ ID NO: 6 - protein, 18 aa, synthetic construct
  VARIANT 4: note = MISC_FEATURE - Xaa is Ser or Leu
  VARIANT 10: note = MISC_FEATURE - Xaa is Thr or Ser
AWIXPYGGSXYYADSVKG

SEQ ID NO: 7 - protein, 9 aa, synthetic construct
RHWPGGFDY

SEQ ID NO: 8 - protein, 25 aa, synthetic construct [sequence omitted]

SEQ ID NO: 9 - protein, 13 aa, synthetic construct
WVRQAPGKGLEWV

SEQ ID NO: 10 - protein, 32 aa, synthetic construct
RFTISADTSKNTAYLQMNSLRAEDTAVYYCAR

SEQ ID NO: 11 - protein, 11 aa, synthetic construct
WGQGTLVTVSS

SEQ ID NO: 12 - protein, 11 aa, synthetic construct
  VARIANT 5: note = MISC_FEATURE - Xaa is Asp or Val
  VARIANT 6: note = MISC_FEATURE - Xaa is Val or Ile
  VARIANT 7: note = MISC_FEATURE - Xaa is Ser or Asn
  VARIANT 9: note = MISC_FEATURE - Xaa is Ala or Phe
  VARIANT 10: note = MISC_FEATURE - Xaa is Val or Leu
RASQXXXTXXA

SEQ ID NO: 13 - protein, 7 aa, synthetic construct
  VARIANT 4: note = MISC_FEATURE - Xaa is Phe or Thr
  VARIANT 6: note = MISC_FEATURE - Xaa is Tyr or Ala
SASXLXS

SEQ ID NO: 14 - protein, 9 aa, synthetic construct
  VARIANT 3: note = MISC_FEATURE - Xaa is Tyr, Gly, Phe, or Ser
  VARIANT 4: note = MISC_FEATURE - Xaa is Leu, Tyr, Phe, or Trp
  VARIANT 5: note = MISC_FEATURE - Xaa is Tyr, Asn, Ala, Thr, Gly, Phe, or Ile
  VARIANT 6: note = MISC_FEATURE - Xaa is His, Val, Pro, Thr, or Ile
  VARIANT 8: note = MISC_FEATURE - Xa is Ala, Trp, Arg, Pro, or Thr
QQXXXXPXT

SEQ ID NO: 15 - protein, 23 aa, synthetic construct
DIQMTQSPSSLSASVGDRVTITC

SEQ ID NO: 16 - protein, 15 aa, synthetic construct
WYQQKPGKAPKLLIY

SEQ ID NO: 17 - protein, 32 aa, synthetic construct
GVPSRFSGSGSGTDFTLTISSLQPEDFATYYC

SEQ ID NO: 18 - protein, 11 aa, synthetic construct
FGQGTKVEIKR

SEQ ID NO: 19 - protein, 10 aa, synthetic construct
GFTFSDSWIH

SEQ ID NO: 20 - protein, 18 aa, synthetic construct
AWISPYGGSTYYADSVKG

SEQ ID NO: 21 - protein, 9 aa, synthetic construct
RHWPGGFDY

SEQ ID NO: 22 - protein, 11 aa, synthetic construct
RASQDVSTAVA

SEQ ID NO: 23 - protein, 7 aa, synthetic construct
SASFLYS

SEQ ID NO: 24 - protein, 9 aa, synthetic construct
QQYLYHPAT

SEQ ID NO: 25 - protein, 122 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 26 - protein, 10 aa, synthetic construct
FGQGTKVEIK

SEQ ID NO: 27 - protein, 30 aa, synthetic construct
EVQLVESGGGLVQPGGSLRLSCAASGFTFS

SEQ ID NO: 28 - protein, 14 aa, synthetic construct
WVRQAPGKGLEWVA

SEQ ID NO: 29 - protein, 15 aa, synthetic construct
WGQGTLVTVSSASTK

SEQ ID NO: 30 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 115 aa, synthetic construct
QSLEESGGRLVKPDETLTITCTVSGIDLSSNGLTWVRQAPGEGLEWIGTINKDASAYYASWAKGRLTISKPSSTKVDLKITSPTTEDTATYFCGRIAFKTGTSIWGPGTLVTVSS

SEQ ID NO: 33 - protein, 112 aa, synthetic construct
AIVMTQTPSPVSAAVGGTVTINCQASESVYSNNYLSWFQQKPGQPPKLLIYLASTLASGVPSRFKGSGSGTQFTLTISGVQCDDAATYYCIGGKSSSTDGNAFGGGTEVVVR

SEQ ID NO: 34 - protein, 7 aa, synthetic construct
SNSAAWN

SEQ ID NO: 35 - protein, 18 aa, synthetic construct
KTYYRFKWYSDYAVSVKG

SEQ ID NO: 36 - protein, 14 aa, synthetic construct
ESTTYDLLAGPFDY

SEQ ID NO: 37 - protein, 17 aa, synthetic construct
KSSQTVLYSSNNKKYLA

SEQ ID NO: 38 - protein, 7 aa, synthetic construct
WASTRES

SEQ ID NO: 39 - protein, 9 aa, synthetic construct
QQYYSTPFT

SEQ ID NO: 40 - protein, 23 aa, synthetic construct
DIVMTQSPDSLAVSLGERATINC

SEQ ID NO: 41 - protein, 15 aa, synthetic construct
WYQQKPGQPPNLLIY

SEQ ID NO: 42 - protein, 32 aa, synthetic construct
GVPDRFSGSGSGTDFTLTISSLQAEDVAVYYC

SEQ ID NO: 43 - protein, 10 aa, synthetic construct
FGPGTKVEIK

SEQ ID NO: 44 - protein, 30 aa, synthetic construct
  VARIANT 1: note = MISC_FEATURE - Xaa is Gln or Glu
XVQLQQSGPGLVKPSQTLSLTCAISGDSVS

SEQ ID NO: 45 - protein, 14 aa, synthetic construct
WIRQSPSRGLEWLG

SEQ ID NO: 46 - protein, 32 aa, synthetic construct
RITINPDTSKNQFSLQLNSVTPEDTAVFYCTR

SEQ ID NO: 47 - protein, 11 aa, synthetic construct
WGQGTLVTVSS

SEQ ID NO: 48 - protein, 30 aa, synthetic construct
EVQLQQSGPGLVKPSQTLSLTCAISGDSVS

SEQ ID NO: 49 - protein, 30 aa, synthetic construct
QVQLQQSGPGLVKPSQTLSLTCAISGDSVS

SEQ ID NO: 50 - protein, 126 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 51 - protein, 126 aa, synthetic construct
QVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 52 - protein, 113 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIK

SEQ ID NO: 53 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 54 - protein, 223 aa, Homo sapiens [sequence omitted]

SEQ ID NO: 55 - protein, 456 aa, synthetic construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 220 aa, synthetic construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC